(19) United States
(12) Patent Application Publication
(10) Pub. No.: US 2004/0023961 A1
(43) Pub. Date: Feb. 5, 2004

(54) Aryl ureas with raf kinase and angiogenisis inhibiting activity

(75) Inventors: Jacques Dumas, Bethany, CT (US); William J. Scott, Guilford, CT (US); James Elting, Madison, CT (US); Holia Hatoum-Makdad, Hamden, CT (US)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER CORPORATION, Pittsburgh, PA

(21) Appl. No.: 10/361,844

(22) Filed: Feb. 11, 2003

Related U.S. Application Data
(60) Provisional application No. 60/354,948, filed on Feb. 11, 2002.

(51) Int. Cl. A61K 31/541; A61K 31/5377; A61K 31/4439; A61K 31/415

(52) U.S. Cl. 514/238.8; 514/341; 514/227.8; 514/406

(57) ABSTRACT

This invention relates to methods of using aryl ureas to treat diseases mediated by raf kinase and diseases mediated by the VEGF induced signal transduction pathway characterized by abnormal angiogenesis or hyperpermeability processes.

### FIELD OF THE INVENTION

This invention relates to methods of treating diseases mediated by the VEGF induced signal transduction pathway and diseases mediated by raf kinase.

### BACKGROUND OF THE INVENTION

The p21^{ras} oncogene, which codes for the ras protein, is a major contributor to the development and progression of human solid cancers and is reported to be mutated in 30% of all human cancers (Bolton et al. Ann. Rep. Med. Chem. 1994, 29, 165-74; Bos. Cancer Res. 1989, 49, 4682-9). In its normal, unmutated form, the ras protein is a key element in the signal transduction cascade involved in cell growth and other fundamental cellular processes (Avruch et al. Trends Biochem. Sci. 1994, 19, 279-83). Biochemically, ras protein is a guanine nucleotide binding protein, and cycles between an active and inactive form. When bound to the guanine nucleotide GTP, ras protein is activated and acts upon downstream target molecules which form part of the signal transduction cascade, such as the enzyme raf kinase. In cancer cells having mutant ras, the ras protein is constitutively active and, therefore, the protein delivers constitutive growth signals to downstream target molecules. This leads to the cancerous growth of the cells which carry these mutations (Magnuson et al. Semin. Cancer Biol. 1994, 5, 247-53).

It has been shown that inhibiting the effect of active ras by inhibiting the downstream target molecules, such as by administration of deactivating antibodies to raf kinase or by co-expression of dominant negative raf kinase or dominant negative MEK, the substrate of raf kinase, can lead to the reversion of a cancer phenotype to the normal growth phenotype have further indicated that inhibition of raf expression by antisense RNA blocks cell proliferation in membrane-associated oncogenes. Similarly, inhibition of raf kinase (by antisense oligodeoxynucleotides) has been correlated in vitro and in vivo with inhibition of the growth of a variety of human tumor types.

Vasculogenesis involves the de novo formation of blood vessels from endothelial cell precursors or angioblasts. The first vascular structures in the embryo are formed by vasculogenesis. Angiogenesis involves the development of capillaries from existing blood vessels, and is the principle mechanism by which organs, such as the brain and the kidney are vascularized. While vasculogenesis is restricted to embryonic development, angiogenesis can occur in the adult, for example during pregnancy, the female cycle, or wound healing.

One major regulator of angiogenesis and vasculogenesis in both embryonic development and some angiogenic-dependent diseases is vascular endothelial growth factor (VEGF; also called vascular permeability factor, VPF). VEGF represents a family of isoforms of mitogens existing in homodimeric forms due to alternative RNA splicing. The VEGF isoforms are highly specific for vascular endothelial cells (for reviews, see: Farrara et al. Endocr. Rev. 1992, 13, 18; Neufield et al. FASEB J. 1999, 13,9).

VEGF expression is induced by hypoxia (Shweiki et al. Nature 1992, 359, 843), as well as by a variety of cytokines and growth factors, such as interleukin-1, interleukin-6, epidermal growth factor and transforming growth factor-□ and -□.

To date VEGF and the VEGF family members have been reported to bind to one or more of three transmembrane receptor tyrosine kinases (Mustonen et al. J. Cell Biol., 1995, 129, 895), VEGF receptor-1 (also known as flt-1 (fms-like tyrosine kinase-1)), VEGFR-2 (also known as kinase insert domain containing receptor (KDR); the murine analogue of KDR is known as fetal liver kinase-1 (flk-1)), and VEGFR-3 (also known as flt-4). KDR and flt-1 have been shown to have different signal transduction properties (Waltenberger et al. J. Biol. Chem. 1994, 269, 26988); Park et al. Oncogene 1995, 10, 135). Thus, KDR undergoes strong ligand-dependant tyrosine phosphorylation in intact cells, whereas flt-1 displays a weak response. Thus, binding to KDR is a critical requirement for induction of the full spectrum of VEGF-mediated biological responses.

In vivo, VEGF plays a central role in vasculogenesis, and induces angiogenesis and permeabilization of blood vessels. Deregulated VEGF expression contributes to the development of a number of diseases that are characterized by abnormal angiogenesis and/or hyperpermeability processes. Regulation of the VEGF-mediated signal transduction cascade will therefore provide a useful mode for control of abnormal angiogenesis and/or hyperpermeability processes.

Angiogenesis is regarded as an absolute prerequisite for growth of tumors beyond about 1-2 mm. Oxygen and nutrients may be supplied to cells in tumor smaller than this limit through diffusion. However, every tumor is dependent on angiogenesis for continued growth after it has reached a certain size. Tumorigenic cells within hypoxic regions of tumors respond by stimulation of VEGF production, which triggers activation of quiescent endothelial cells to stimulate new blood vessel formation. (Shweiki et al. Proc. Nat'l. Acad. Sci., 1995, 92, 768). In addition, VEGF production in tumor regions where there is no angiogenesis may proceed through the ras signal transduction pathway (Grugel et al. J. Biol. Chem., 1995, 270, 25915; Rak et al. Cancer Res. 1995, 55, 4575). In situ hybridization studies have demonstrated VEGF mRNA is strongly upregulated in a wide variety of human tumors, including lung (Mattern et al. Br. J. Cancer 1996, 73, 931), thyroid (Viglietto et al. Oncogene 1995, 11, 1569), breast (Brown et al. Human Pathol. 1995, 26, 86), gastrointestinal tract (Brown et al. Cancer Res. 1993, 53, 4727; Suzuki et al. Cancer Res. 1996, 56, 3004), kidney and bladder (Brown et al. Am. J. Pathol. 1993, 1431, 1255), ovary (Olson et al. Cancer Res. 1994, 54, 1255), and cervical (Guidi et al. J. Nat'l Cancer Inst. 1995, 87, 12137) carcinomas, as well as angiosacroma (Hashimoto et al. Lab. Invest. 1995, 73, 859) and several intracranial tumors (Plate et al. Nature 1992, 359, 845; Phillips et al. Int. J. Oncol. 1993, 2, 913; Berkman et al. J. Clin. Invest., 1993, 91, 153). Neutralizing monoclonal antibodies to KDR have been shown to be efficacious in blocking tumor angiogenesis (Kim et al. Nature 1993, 362, 841; Rockwell et al. Mol. Cell. Differ. 1995, 3, 315).

Overexpression of VEGF, for example under conditions of extreme hypoxia, can lead to intraocular angiogenesis, resulting in hyperproliferation of blood vessels, leading eventually to blindness. Such a cascade of events has been observed for a number of retinopathies, including diabetic retinopathy, ischemic retinal-vein occlusion, retinopathy of prematurity (Aiello et al. New Engl. J. Med. 1994, 331, 1480; Peer et al. Lab. Invest. 1995, 72, 638), and age-related macular degeneration (AMD; see, Lopez et al. Invest. Opththalmol. Vis. Sci. 1996, 37, 855).

In rheumatoid arthritis (RA), the in-growth of vascular pannus may be mediated by production of angiogenic factors. Levels of immunoreactive VEGF are high in the synovial fluid of RA patients, while VEGF levels were low in the synovial fluid of patients with other forms of arthritis of with degenerative joint disease (Koch et al. J. Immunol. 1994, 152, 4149). The angiogenesis inhibitor AGM-170 has been shown to prevent neovascularization of the joint in the rat collagen arthritis model (Peacock et al. J. Exper. Med. 1992, 175, 1135).

Increased VEGF expression has also been shown in psoriatic skin, as well as bullous disorders associated with subepidermal blister formation, such as bullous pemphigoid, erythema multiforme, and dermatitis herpetiformis (Brown et al. J. Invest. Dermatol. 1995, 104, 744).

Because inhibition of KDR leads to inhibition of VEGF-mediated angiogenesis and permeabilization, KDR inhibitors will be useful in treatment of diseases characterized by abnormal angiogenesis and/or hyperpermeability processes, including the above listed diseases.

The p21^{ras} oncogene is a major contributor to the development and progression of human solid cancers and is mutated in 30%/o of all human cancers (Boltoii et al. Ann. Rep. Med. Chem. 1994, 29, 165-74; Bos. Chancer Res. 1989, 49, 4689-9). In its normal, unmutated form, the ras protein is a key element of the signal transduction cascade directed by growth factor receptors in almost all tissues (Avruch et al. Trends Biochem. Sci. 1994, 19, 279-83). Biochemically, ras is a guanine nucleotide binding protein, and cycling between a GTP-bound activated and a GDP-bound resting form is strictly controlled by ras' endogenous GTPase activity and other regulatory proteins. In the ras mutants in cancer cells, the endogenous GTPase activity is alleviated and, therefore, the protein delivers constitutive growth signals to downstream effectors such as the enzyme raf kinase. This leads to the cancerous growth of the cells which carry these mutants (Magnuson et al. Semin. Cancer Biol. 1994, 5, 247-53). It has been shown that inhibiting the effect of active ras by inhibiting the raf kinase signaling pathway by administration of deactivating antibodies to raf kinase or by co-expression of dominant negative raf kinase or dominant negative MEK, the substrate of raf kinase, leads to the reversion of transformed cells to the normal growth phenotype (see: Daum et al. Trends Biochem. Sci. 1994, 19, 474-80; Fridman et al. J. Biol. Chem. 1994, 269, 30105-8. Kolch et al. (Nature 1991, 349, 426-28) have further indicated that inhibition of raf expression by antisense RNA blocks cell proliferation in membrane-associated oncogenes. Similarly, inhibition of raf kinase (by antisense oligodeoxynucleotides) has been correlated in vitro and in vivo with inhibition of the growth of a variety of human tumor types (Monia et al., Nat. Med. 1996, 2, 668-75).

### SUMMARY OF THE INVENTION

The current invention provides methods of-using the diaryl ureas listed below. The compounds of the present invention are useful as therapeutic compounds for the treatment and prevention of cancer and also angiogenesis disorders.

As inhibitors of raf kinase, these compounds are useful in the treatment of tumors and/or cancerous growth mediated by raf kinase. In particular, the compounds are useful in the treatment of human or animal solid cancers, e.g., murine cancer, carcinomas (e.g., of the lungs, pancreas, thyroid, bladder or colon), myeloid disorders (e.g., myeloid leukemia) or adenomas (e.g., villous colon adenoma).

Since the enzyme is a downstream effector of p21^{ras}, the compounds of this invention find use in pharmaceutical compositions for human or veterinary use where inhibition of the raf kinase pathway is indicated, e.g., in the treatment of tumors and/or cancerous cell growth mediated by raf kinase. In particular, the compounds are useful in the treatment of human or animal solid cancers, e.g., murine cancer, since the progression of these cancers is dependent upon the ras protein signal transduction cascade and therefore susceptible to treatment by interruption of the cascade, i.e., by inhibiting raf kinase.

As inhibitors of KDR, these compounds are useful for treating diseases in humans and/or other mammals which are mediated by the VEGF induced signal transduction pathway, including those characterized by abnormal angiogenesis or hyperpermeability processes.

The present invention, therefore, provides methods for the treatment of cancer, and angiogenesis disorders in humans and/or other mammals, wherein a compound of this invention is administered, or a salt, prodrug or isolated stereoisomer thereof. These methods comprise administering a compound of this invention to a human or other mammal with a disease characterized by a hyper-proliferative disorder such as cancer or a disease characterized by abnormal angiogenesis or hyperpermiability processes.

The compounds listed below, including all stereoisomeric forms thereof (both isolated and in mixtures), the salts thereof, and prodrugs thereof are collectively referred to herein as the “compounds of the invention.”

| | |
|---|---|
| 1 | 1-[5-tert-butyl-2-(2-methylpyridin-5-yl)-2H-pyrazol-3-yl]-3-(4- |
| | chlorophenyl)urea |
| 2 | 1-(5-tert-butyl-2-p-tolyl-2H-pyrazol-3-yl)-3-(4- |
| | methoxynaphthalen-1-yl)urea |
| 3 | 1-[5-tert-butyl-2-(3,4-dimethylphenyl)-2H-pyrazol-3-yl]-3-(4- |
| | fluorophenyl)urea |
| 4 | 1-[5-tert-butyl-2-(pyridin-3-yl)-2H-pyrazol-3-yl]-3-(4- |
| | cyanonaphthalen-1-yl)urea |
| 5 | 1-[5-tert-butyl-2-p-tolyl-2H-pyrazol-3-yl]-3-[4-(2-morpholin-4- |
| | ylethoxy)naphthalen-1-yl]-urea |
| 6 | 1-[5-tert-butyl-2-p-tolyl-2H-pyrazol-3-yl]-3-[4-(2-(cis-2,6- |
| | dimethylmorpholin-4-yl)ethoxy)naphthalen-1-yl]-urea |
| 7 | 1-[5-tert-butyl-2-p-tolyl-2H-pyrazol-3-yl]-3-[4-(2-(trans-2,6- |
| | dimethylmorpholin-4-yl)ethoxy)naphthalen-1-yl]-urea |
| 8 | 1-[5-tert-butyl-2-p-tolyl-2H-pyrazol-3-yl]-3-[4-(2-(2- |
| | (methoxymethyl)morpholin-4-yl)ethoxy)naphthalen-1-yl]-urea |
| 9 | 1-[5-tert-butyl-2-p-tolyl-2H-pyrazol-3-yl]-3-[4-(2- |
| | (morpholin-4-yl)-2-oxoethoxy)naphthalen-1-yl]-urea |
| 10 | 1-[5-tert-butyl-2-p-tolyl-2H-pyrazol-3-yl]-3-[4-(2- |
| | (morpholin-4-yl)-2-methylethoxy)naphthalen-1-yl]-urea |
| 11 | 1-[5-tert-butyl-2-p-tolyl-2H-pyrazol-3-yl]-3-[4-(2- |
| | (morpholin-4-yl)-1-methylethoxy)naphthalen-1-yl]-urea |
| 12 | 1-[5-tert-butyl-2-p-tolyl-2H-pyrazol-3-yl]-3-[4-(2-thiomorpholin-4- |
| | ylethoxy)naphthalen-1-yl]-urea |
| 13 | 1-[5-tert-butyl-2-p-tolyl-2H-pyrazol-3-yl]-3-[4-(2-(1- |
| | oxothiomorpholin-4-yl)ethoxy)naphthalen-1-yl]-urea |
| 14 | 1-[5-tert-butyl-2-p-tolyl-2H-pyrazol-3-yl]-3-[4-(2-morpholin-4-yl- |
| | ethoxy)-3-methylnaphthalen-1-yl]-urea |
| 15 | 1-[5-tert-butyl-2-p-tolyl-2H-pyrazol-3-yl]-3-[4-(2-(morpholin-4-yl- |
| | carbonyloxo)ethoxy)naphthalen-1-yl]-urea |
| 16 | 1-[5-tert-butyl-2-p-tolyl-2H-pyrazol-3-yl]-3-[4-(2- |
| | (tetrahydropyran-4-yl)ethoxy)naphthalen-1-yl]-urea |
| 17 | 1-[5-tert-butyl-2-p-tolyl-2H-pyrazol-3-yl]-3-[4-(2-(1-oxo- |
| | tetrahydrothiophen-3-yl)ethoxy)naphthalen-1-yl]-urea |
| 18 | 1-[5-tert-butyl-2-p-tolyl-2H-pyrazol-3-yl]-3-[4-(3-morpholin-4-yl- |
| | propyl)naphthalen-1-yl]-urea |
| 19 | 1-[5-tert-butyl-2-p-tolyl-2H-pyrazol-3-yl]-3-[4-(3-morpholin-4-yl- |
| | methyl)naphthalen-1-yl]-urea |
| 20 | 1-[5-tert-butyl-2-p-tolyl-2H-pyrazol-3-yl]-3-[4-(2-pyridin-4-yl- |
| | ethyl)naphthalen-1-yl]-urea |
| 21 | 1-[5-tert-butyl-2-p-tolyl-2H-pyrazol-3-yl]-3-[4-(3-(morpholin-4- |
| | yl)propyn-1-yl)naphthalen-1-yl]-urea |
| 22 | 1-[5-tert-butyl-2-p-tolyl-2H-pyrazol-3-yl]-3-[4-(3- |
| | (tetrahydropyran-2-yl-oxy)propyn-1-yl)naphthalen-1-yl]-urea |
| 23 | 1-[5-tert-butyl-2-p-tolyl-2H-pyrazol-3-yl]-3-[4-(3- |
| | (tetrahydropyran-2-yl-oxy)butyn-1-yl)naphthalen-1-yl]-urea |
| 24 | 1-[5-tert-butyl-2-p-tolyl-2H-pyrazol-3-yl]-3-[4-(3-(piperidin-1- |
| | yl)propyn-1-yl)naphthalen-1-yl]-urea |
| 25 | 1-[5-tert-butyl-2-p-tolyl-2H-pyrazol-3-yl]-3-[4-(3-(2- |
| | methoxymethylmorpholin-4-yl)propyn-1-yl)naphthalen-1-yl]-urea |
| 26 | 1-[5-tert-butyl-2-p-tolyl-2H-pyrazol-3-yl]-3-[4-(pyridin-4-yl- |
| | methoxy)naphthalen-1-yl]-urea |
| 27 | 1-[5-tert-butyl-2-p-tolyl-2H-pyrazol-3-yl]-3-[4-(2-pyridin-4-yl- |
| | ethoxy)naphthalen-1-yl]-urea |
| 28 | 1-[5-tert-butyl-2-p-tolyl-2H-pyrazol-3-yl]-3-[4-(3-pyridin-4-yl- |
| | propoxy)naphthalen-1-yl]-urea |
| 29 | 1-[5-tert-butyl-2-p-tolyl-2H-pyrazol-3-yl]-3-[4-(2-imidazol-1-yl- |
| | ethoxy)naphthalen-1-yl]-urea |
| 30 | 1-[5-tert-butyl-2-p-tolyl-2H-pyrazol-3-yl]-3-[4-(2-(3,4- |
| | dimethoxyphenyl)-ethoxy)naphthalen-1-yl]-urea |
| 31 | 1-[5-tert-butyl-2-p-tolyl-2H-pyrazol-3-yl]-3-[4-(pyridin-4-yl- |
| | methylamino)naphthalen-1-yl]-urea |
| 32 | 1-[5-isopropyl-2-phenyl-2H-pyrazol-3-yl]-3-[4-(2-morpholin-4-yl- |
| | ethoxy)naphthalen-1-yl]-urea |
| 33 | 1-[5-cyclohexyl-2-phenyl-2H-pyrazol-3-yl]-3-[4-(2-morpholin-4-yl- |
| | ethoxy)naphthalen-1-yl]-urea |
| 34 | 1-[5-(2,2,2-trifluoroethyl)-2-phenyl-2H-pyrazol-3-yl]-3-[4-(2- |
| | morpholin-4-yl-ethoxy)naphthalen-1-yl]-urea |
| 35 | 1-[5-(1-methylcycloprop-1-yl)-2-phenyl-2H-pyrazol-3-yl]-3-[4-(2- |
| | morpholin-4-yl-ethoxy)naphthalen-1-yl]-urea |
| 36 | 1-[5-(1-methylcyclohex-1-yl)-2-phenyl-2H-pyrazol-3-yl]-3-[4-(2- |
| | morpholin-4-yl-ethoxy)naphthalen-1-yl]-urea |
| 37 | 1-[5-tert-butyl-2-methyl-2H-pyrazol-3-yl]-3-[4-(2-morpholin-4-yl- |
| | ethoxy)naphthalen-1-yl]-urea |
| 38 | 1-[5-tert-butyl-2-(4-chlorophenyl)-2H-pyrazol-3-yl]-3-[4-(2- |
| | morpholin-4-yl-ethoxy)naphthalen-1-yl]-urea |
| 39 | 1-[5-tert-butyl-2-butyl-2H-pyrazol-3-yl]-3-[4-(2-morpholin-4-yl- |
| | ethoxy)naphthalen-1-yl]-urea |
| 40 | 1-[5-tert-butyl-2-(4-methyl-3-carbamylphenyl)-2H-pyrazol-3-yl]-3- |
| | [4-(2-morpholin-4-yl-ethoxy)naphthalen-1-yl]-urea |
| 41 | 1-[5-tert-butyl-2-(4-methyl-3-(morpholin-4-yl)methylphenyl)-2H- |
| | pyrazol-3-yl]-3-[4-(2-morpholin-4-yl-ethoxy)naphthalen-1-yl]-urea |
| 42 | 1-[5-tert-butyl-2-(4-methyl-3-dimethylaminomethylphenyl)-2H- |
| | pyrazol-3-yl]-3-[4-(2-morpholin-4-yl-ethoxy)naphthalen-1-yl]-urea |
| 43 | 1-[5-tert-butyl-2-(3-dimethylaminomethylphenyl)-2H-pyrazol- |
| | 3-yl]-3-[4-(2-morpholin-4-yl-ethoxy)naphthalen-1-yl]-urea |
| 44 | 1-[5-tert-butyl-2-(2-chloropyridin-5-yl)-2H-pyrazol-3-yl]-3-[4-(2- |
| | morpholin-4-yl-ethoxy)naphthalen-1-yl]-urea |
| 45 | 1-[5-tert-butyl-2-(2-methylpyridin-5-yl)-2H-pyrazol-3-yl]-3-[4-(2- |
| | morpholin-4-yl-ethoxy)naphthalen-1-yl]-urea |
| 46 | 1-[5-tert-butyl-2-(2-methoxypyridin-5-yl)-2H-pyrazol-3-yl]-3-[4-(2- |
| | morpholin-4-yl-ethoxy)naphthalen-1-yl]-urea |
| 47 | 1-[5-tert-butyl-2-(pyridin-3-yl)-2H-pyrazol-3-yl]-3-[4- |
| | (2-morpholin-4-yl-ethoxy)naphthalen-1-yl]-urea |
| 48 | 1-[5-tert-butyl-2-(2-methylpyridin-5-yl)-2H-pyrazol-3-yl]-3-[4-(2- |
| | pyridin-4-yl-ethoxy)naphthalen-1-yl]-urea |
| 49 | 1-[5-tert-butyl-2-(2-methylpyridin-5-yl)-2H-pyrazol-3-yl]-3-[4-(2- |
| | (trans-2,6-dimethylmorpholin-4-yl)ethoxy)naphthalen-1-yl]-urea |
| 50 | 1-[5-tert-butyl-2-(2-methylpyridin-5-yl)-2H-pyrazol-3-yl]-3-[4-(3- |
| | morpholin-4-yl-propyn-1-yl)naphthalen-1-yl]-urea |
| 51 | 1-[5-(2-hydroxy-1,1-dimethyl-ethyl)-2-p-tolyl-2H-pyrazol-3- |
| | yl]-3-[4-(2-morpholin-4-yl-ethoxy)naphthalen-1-yl]-urea |
| 52 | 1-[5-tert-butyl-2-(3-hydroxy-4-methyl-phenyl)-2H-pyrazol-3- |
| | yl]-3-[4-(2-morpholin-4-yl-ethoxy)naphthalen-1-yl]-urea |
| 53 | 1-[5-tert-butyl-2-(4-hydroxymethyl-phenyl)-2H-pyrazol-3- |
| | yl]-3-[4-(2-morpholin-4-yl-ethoxy)naphthalen-1-yl]-urea |
| 54 | 1-[5-tert-butyl-2-p-tolyl-2H-pyrazol-3-yl]-3-{4-[2- |
| | (3-oxo-morpholin-4-yl)-ethoxy]naphthalen-1-yl}-urea |
| 55 | 1-[5-tert-butyl-2-p-tolyl-2H-pyrazol-3-yl]-3-{4-[2-(4-hydroxy- |
| | morpholin-4-yl)-ethoxy]naphthalen-1-yl}-urea |
| 56 | 1-[5-(2-hydroxy-1,1-dimethyl-ethyl)-2-(6-methyl-pyridin-3-yl)-2H- |
| | pyrazol-3-yl]-3-[4-(2-morpholin-4-yl-ethoxy)naphthalen-1-yl]-urea |
| 57 | 1-[5-tert-butyl-2-(1-hydroxy-6-methyl-pyridin-3-yl)-2H-pyrazol-3- |
| | yl]-3-[4-(2-morpholin-4-yl-ethoxy)naphthalen-1-yl]-urea |
| 58 | 1-[5-tert-butyl-2-(6-methyl-pyridin-3-yl)-2H-pyrazol-3-yl]-3-{4-[2- |
| | (4-hydroxy-morpholin-4-yl)-ethoxy]naphthalen-1-yl}-urea |
| 59 | 1-[5-(2-hydroxy-1,1-dimethyl-ethyl)-2-(6-methyl-pyridin-3-yl)-2H- |
| | pyrazol-3-yl]-3-[4-(2-pyridin-4-yl-ethoxy)naphthalen-1-yl]-urea |
| 60 | 1-[5-tert-butyl-2-(6-methyl-pyridin-3-yl)-2H-pyrazol-3-yl]-3-[4-(2- |
| | hydroxy-2-pyridin-4-yl-ethoxy)naphthalen-1-yl]-urea |
| 61 | 1-[5-tert-butyl-2-(6-methyl-pyridin-3-yl)-2H-pyrazol-3-yl]-3-{4-[2- |
| | (1-hydroxy-2-pyridin-4-yl)-ethoxy]naphthalen-1-yl}-urea |
| 62 | 1-[5-(2-hydroxy-1,1-dimethyl-ethyl)-2-p-tolyl-2H-pyrazol-3- |
| | yl]-3-{4-[2-(1-oxo-thiomorpholin-4-yl)- |
| | ethoxy]naphthalen-1-yl}-urea |
| 63 | 1-[5-tert-butyl-2-(4-hydroxymethyl-phenyl)-2H-pyrazol-3-yl]-3-{4- |
| | [2-(1-oxo-thiomorpholin-4-yl)-ethoxy]naphthalen-1-yl}-urea |
| 64 | 1-[5-tert-butyl-2-p-tolyl-2H-pyrazol-3-yl]-3-{4-[2-(1,3-dioxo- |
| | thiomorpholin-4-yl)-ethoxy]naphthalen-1-yl}-urea |
| 65 | 1-[5-(2-hydroxy-1,1-dimethyl-ethyl)-2-methyl-2H-pyrazol-3- |
| | yl]-3-[4-(2-morpholin-4-yl-ethoxy)-naphthalen-1-yl]-urea |
| 66 | 1-[5-tert-butyl-2-methyl-2H-pyrazol-3-yl]-3-{4-[2-(4-hydroxy- |
| | morpholin-4-yl)-ethoxy]-naphthalen-1-yl}-urea |
| 67 | 1-[5-tert-butyl-2-p-tolyl-2H-pyrazolyl-3-yl]-3-[4-(4- |
| | (morpholin-4-yl-methyl)phenyl)naphthalen-1-yl]urea |
| 68 | 1-[5-tert-butyl-2-p-tolyl-2H-pyrazolyl-3-yl]-3-[4-(4- |
| | (2-(morpholin-4-yl)ethyl)phenyl)naphthalen-1-yl]urea |
| 69 | 1-[5-tert-butyl-2-p-tolyl-2H-pyrazolyl-3-yl]-3-[4-(3- |
| | (morpholin-4-yl-methyl)phenyl)naphthalen-1-yl]urea |
| 70 | 1-[5-tert-butyl-2-p-tolyl-2H-pyrazolyl-3-yl]-3-[4-(6-(morpholin-4- |
| | ylmethyl-pyridin-3-yl)naphthalen-1-yl]urea |
| 71 | 1-[5-tert-butyl-2-p-tolyl-2H-pyrazolyl-3-yl]-3-[4-(5-(morpholin-4- |
| | ylmethyl-pyridin-2-yl)naphthalen-1-yl]urea |
| 72 | 1-[5-tert-butyl-2-p-tolyl-2H-pyrazolyl-3-yl]-3-[4-(5-(morpholin-4- |
| | ylmethyl-fur-2-yl)naphthalen-1-yl]urea |
| 73 | 1-[5-tert-butyl-2-(6-methyl-pyridin-3-yl)-2H-pyrazolyl-3- |
| | yl]-3-[4-(6-(morpholin-4-ylmethyl-pyridin-3-yl)naphthalen- |
| | 1-yl]urea |
| 74 | 1-[5-tert-butyl-2-methyl-2H-pyrazolyl-3-yl]-3-[4-(6-morpholin-4- |
| | ylmethyl-pyridin-3-yl)naphthalen-1-yl]urea |
| 75 | 1-[5-tert-butyl-2-p-tolyl-2H-pyrazolyl-3-yl]-3-[4-(5-(morpholin-4- |
| | yl-methyl)pyridin-2-yl)naphthalen-1-yl]urea |
| 76 | 1-[5-tert-butyl-2-p-tolyl-2H-pyrazolyl-3-yl]-3-[4-(6-(morpholin-4- |
| | yl-methyl)pyridin-3-yl)naphthalen-1-yl]urea |
| 77 | 1-[5-tert-butyl-2-(6-methyl-pyridin-3-yl)-2H-pyrazolyl-3-yl]- |
| | 3-[4-(3-(2-(pyridin-2-yl)ethylamino)cyclohexenyl)- |
| | naphthalen-1-yl]urea |
| 78 | 1-[5-tert-butyl-2-p-tolyl-2H-pyrazolyl-3-yl]-3-[4-(4-(pyridin-3-yl- |
| | methylaminomethyl)phenyl)naphthalen-1-yl]urea |
| 79 | 1-[5-tert-butyl-2-(6-methyl-pyridin-3-yl)-2H-pyrazolyl-3- |
| | yl]-3-[4-(4-(morpholin-4-yl-methyl)phenyl)naphthalen- |
| | 1-yl]urea |
| 80 | 1-[5-tert-butyl-2-(6-methyl-pyridin-3-yl)-2H-pyrazolyl-3- |
| | yl]-3-[4-(6-(4-(hydroxybutylamino)pyridin-3-yl)- |
| | naphthalen-1-yl]urea |
| 81 | 1-[5-tert-butyl-2-(4-methyl-3-carbamylphenyl)-2H-pyrazolyl-3- |
| | yl]-3-[4-(6-(morpholin-4-yl-methyl)pyridin-3-yl)naphthalen- |
| | 1-yl]urea |
| 82 | 1-[5-tert-butyl-2-(6-methyl-pyridin-3-yl)-2H-pyrazolyl-3-yl]-3- |
| | [4-(4-(3-hydroxypiuperidin-1-yl-methyl)phenyl)naphthalen-1- |
| | yl]urea |
| 83 | 1-[5-tert-butyl-2-(6-methyl-pyridin-3-yl)-2H-pyrazolyl-3-yl]-3- |
| | [4-(4-(4-hydroxymorpholin-4-yl-methyl)phenyl)naphthalen-1- |
| | yl]urea |
| 84 | 1-[5-tert-butyl-2-(6-methyl-pyridin-3-yl)-2H-pyrazolyl-3-yl]-3- |
| | [4-(3-(morpholin-4-yl-methyl)cyclohexenyl)naphthalen-1- |
| | yl]urea |
| 85 | 1-[5-tert-butyl-2-(6-methyl-pyridin-3-yl)-2H-pyrazolyl-3- |
| | yl]-3-[4-(4-(tetrahydrofuran-3-yl-methyl)-3- |
| | hydroxyphenyl)naphthalen-1-yl]urea |
| 86 | 1-[5-tert-butyl-2-(6-methyl-pyridin-3-yl)-2H-pyrazolyl-3- |
| | yl]-3-[4-(4-(N,N-di-(2- |
| | methoxyethyl)aminomethyl)phenyl)naphthalen-1-yl]urea |
| 87 | 1-[5-tert-butyl-2-(6-methyl-pyridin-3-yl)-2H-pyrazolyl-3- |
| | yl]-3-[4-(6-(cyanopropoxy)pyridin-3-yl)naphthalen-1- |
| | yl]urea |
| 88 | 1-[5-tert-butyl-2-(6-methyl-pyridin-3-yl)-2H-pyrazolyl-3- |
| | yl]-3-[4-(4-morpholin-4-yl-methyl- |
| | piperidinyl)naphthalen-1-yl]urea |
| 89 | 1-[5-tert-butyl-2-(6-methyl-pyridin-3-yl)-2H-pyrazolyl-3- |
| | yl]-3-[4-(4-(N,N-di-(2- |
| | cyanoethyl)aminomethyl)phenyl)naphthalen-1-yl]urea |
| 90 | 1-[5-tert-butyl-2-(6-methyl-pyridin-3-yl)-2H-pyrazolyl-3- |
| | yl]-3-[4-(4-(furan-2-yl-methyl)-3- |
| | hydroxyphenyl)naphthalen-1-yl]urea |
| 91 | 1-[5-tert-butyl-2-(6-methyl-pyridin-3-yl)-2H-pyrazolyl-3- |
| | yl]-3-[4-(4-(thiomorpholin-4-yl- |
| | methyl)phenyl)naphthalen-1-yl]urea |
| 92 | 1-[5-tert-butyl-2-(6-methyl-pyridin-3-yl)-2H-pyrazolyl-3- |
| | yl]-3-[4-(4-(3-carboxamidopiperidin-1-yl- |
| | methyl)phenyl)naphthalen-1-yl]urea |
| 93 | 1-[5-tert-butyl-2-(6-methyl-pyridin-3-yl)-2H-pyrazolyl-3- |
| | yl]-3-[4-(4-(2-methyl-3-oxo-piperzin-1-yl- |
| | methyl)phenyl)naphthalen-1-yl]urea |
| 94 | 1-[5-tert-butyl-2-(2-methylpyrimidin-5-yl)-2H-pyrazolyl-3-yl]-3-[4- |
| | (6-(morpholin-4-yl-methyl)pyridin-3-yl)naphthalen-1-yl]urea |
| 95 | 1-[5-tert-butyl-2-(6-methyl-pyridin-3-yl)-2H-pyrazolyl-3- |
| | yl]-3-[4-(6-(4-hydroxybutyloxy)pyridin-3- |
| | yl)naphthalen-1-yl]urea |
| 96 | 1-[3-tert-butyl-1′H-[1,4′]bipyrazol-5-yl]-3-[4-(6-(morpholin-4-yl- |
| | methyl)pyridin-3-yl)naphthalen-1-yl]urea |
| 97 | 1-[5-tert-butyl-2-2H-pyrazolyl-3-yl]-3-[4-(6- |
| | (tetrahydrothiopyran-4-yl-amino)pyridin-3- |
| | yl)naphthalen-1-yl]urea |
| 98 | 1-[5-tert-butyl-2-(2-cyanoethyl)-2H-pyrazolyl-3-yl]-3-[4-(6- |
| | (morpholin-4-yl-methyl)pyridin-3-yl)naphthalen-1-yl]urea |
| 99 | 1-[5-tert-butyl-2-(6-methyl-pyridin-3-yl)-2H-pyrazolyl-3- |
| | yl]-3-[4-(6-(2,6-dim ethylmorpholin-4-yl-methyl)pyridin-3- |
| | yl)naphthalen-1-yl]urea |
| 100 | 1-[5-tert-butyl-2-(2-methoxypyridin-5-yl)-2H-pyrazolyl-3- |
| | yl]-3-[4-(6-(morpholin-4-yl-methyl)pyridin-3- |
| | yl)naphthalen-1-yl]urea |
| 101 | 1-[5-tert-butyl-2-(2-aminopyridin-5-yl)-2H-pyrazolyl-3-yl]-3-[4-(6- |
| | (morpholin-4-yl-methyl)pyridin-3-yl)naphthalen-1-yl]urea |
| 102 | 1-[5-tert-butyl-2-(6-methyl-pyridin-3-yl)-2H-pyrazolyl-3- |
| | yl]-3-[4-(6-(morpholin-4-yl-4-carbonyl)pyridin-3- |
| | yl)naphthalen-1-yl]urea |
| 103 | 1-[5-tert-butyl-2-(6-methyl-pyridin-3-yl)-2H-pyrazolyl-3- |
| | yl]-3-[4-(6-(2-oxa-5-aza-bicyclo[2.2.1]hept-5-yl- |
| | methyl)pyridin-3-yl)naphthalen-1-yl]urea |
| 104 | 1-[5-tert-butyl-2-(6-methyl-pyridin-3-yl)-2H-pyrazolyl-3- |
| | yl]-3-[4-(4-(N-(2-cyanoethyl)-N-(pyridin-3- |
| | ylmethyl)aminomethyl)phenyl)naphthalen-1-yl]urea |
| 105 | 1-[5-tert-butyl-2-(6-methyl-pyridin-3-yl)-2H-pyrazolyl-3- |
| | yl]-3-[4-(4-(N-(2-cyanoethyl)-N-(tetrahydrofuran-2-yl- |
| | methyl)aminomethyl)phenyl)naphthalen-1-yl]urea |
| 106 | 1-[5-tert-butyl-2-(6-methyl-pyridin-3-yl)-2H-pyrazolyl-3- |
| | yl]-3-[4-(6-(morpholin-4-yl-methyl)-4-methoxypyridin-3- |
| | yl)naphthalen-1-yl]urea |
| 107 | 1-[5-tert-butyl-2-(6-methyl-pyridin-3-yl)-2H-pyrazolyl-3- |
| | yl]-3-[4-(6-(1-morpholin-4-yl-propyl)pyridin-3- |
| | yl)naphthalen-1-yl]urea |
| 108 | 1-[3-tert-butyl-1′-methyl-1′H-[1,4′]bipyrazol-5-yl]-3-[4-(6- |
| | (morpholin-4-yl-methyl)pyridin-3-yl)naphthalen-1-yl]urea |
| 109 | 1-[5-tert-butyl-2-(6-methyl-pyridin-3-yl)-2H-pyrazolyl-3- |
| | yl]-3-[4-(6-(1-oxo-tetrahydrothiopyran-4-yl- |
| | amino)pyridin-3-yl)naphthalen-1-yl]urea |
| 110 | 1-[5-tert-butyl-2-(6-methyl-pyridin-3-yl)-2H-pyrazolyl-3- |
| | yl]-3-[4-(6-(tetrahydropyran-4-yl-amino)pyridin-3- |
| | yl)naphthalen-1-yl]urea |
| 111 | 1-[5-tert-butyl-2-(6-methyl-pyridin-3-yl)-2H-pyrazolyl-3- |
| | yl]-3-[4-(5-(tetrahydrothiopyran-4-yl-aminbo)pyrazin-2- |
| | yl)naphthalen-1-yl]urea |
| 112 | 1-[5-tert-butyl-2-(6-methyl-pyridin-3-yl)-2H-pyrazolyl-3- |
| | yl]-3-[4-(6-(metrhylcarbonylamino)pyridin-3- |
| | yl)naphthalen-1-yl]urea |
| 113 | 1-[5-tert-butyl-1′-(3-methylsulfanylpropyl)-1′H-[1,4′]bipyrazol-5- |
| | yl]-3-[4-(6-(morpholin-4-yl-methyl)pyridin-3-yl)naphthalen-1- |
| | yl]urea |
| 114 | 1-[5-tert-butyl-2-(2-methylpyrimidin-5-yl)-2H-pyrazolyl-3-yl]-3-[4- |
| | (6-(1-oxo-thiomorpholin-4-yl-methyl)pyridin-3-yl)naphthalen-1- |
| | yl]urea |
| 115 | 1-[5-tert-butyl-2-(2-methylpyrimidin-5-yl)-2H-pyrazolyl-3-yl]-3-[4- |
| | (6-(tetrahydropyran-4-ylamino)pyridin-3-yl)naphthalen-1-yl]urea |
| 116 | 1-[5-tert-butyl-2-(2-methylthiopyrimidin-5-yl)-2H-pyrazolyl-3- |
| | yl]-3-[4-(6-(morpholin-4-yl-methyl)pyridin-3-yl)naphthalen-1- |
| | yl]urea |
| 117 | 1-[5-tert-butyl-2-(2-aminopyrimidin-5-yl)-2H-pyrazolyl-3-yl]-3-[4- |
| | (6-(morpholin-4-yl-methyl)pyridin-3-yl)naphthalen-1-yl]urea |
| 118 | 1-[3-tert-butyl-1′-methyl-1′H-[1,4;]bipyrazol-5-yl]-3-[4-(6- |
| | (morpholin-4-yl-methyl)phenyl)naphthalen-1-yl]-urea |
| 119 | 1-[5-tert-butyl-2-p-tolyl-2H-pyrazolyl-3-yl]-3-[4-(6-(1-oxo- |
| | tetrahydrothiopyran-4-yl-amino)pyridin-3-yl)naphthalen-1-yl]urea |
| 120 | 1-[5-tert-butyl-2-p-tolyl-2H-pyrazolyl-3-yl]-3-[4-(6-(thiomorpholin- |
| | 4-yl-methyl)pyridin-3-yl)naphthalen-1-yl]urea |
| 121 | 1-[5-tert-butyl-2-p-tolyl-2H-pyrazolyl-3-yl]-3-[4-(2- |
| | (morpholin-4-yl-carbonyl)pyrimidin-5-yl)naphthalen-1- |
| | yl]urea |
| 122 | 1-[5-tert-butyl-2-p-tolyl-2H-pyrazolyl-3-yl]-3-[4-(4- |
| | (morpholin-4-yl-methyl)pyrimidin-5-yl)naphthalen-1- |
| | yl]urea |
| 123 | 1-[5-tert-butyl-2-p-tolyl-2H-pyrazolyl-3-yl]-3-[4-(6-(1-oxo- |
| | thiomorpholin-4-yl-methyl)pyridin-3-yl)naphthalen-1-yl]urea |
| 124 | 1-[5-tert-butyl-2-(2-methylpyrimidin-5-yl)-2H-pyrazolyl-3-yl]-3-[4- |
| | (2-(morpholin-4-yl-methyl)pyrimidin-5-yl)naphthalen-1-yl]urea |
| 125 | 1-[2-tert-butyl-5-methyl-pyridin-4-yl]-3-[4-(6-(morpholin-4-yl- |
| | methyl-pyridin-3-yl)naphthalen-1-yl]urea |
| 126 | 1-[3-tert-butyl-phenyl]-3-[4-(6-(morpholin-4-yl-methyl)pyridin-3- |
| | yl)naphthalen-1-yl]urea |
| 127 | 1-[4-methyl-biphenyl-3-yl]-3-[4-(6-(morpholin-4-yl-methyl- |
| | pyridin-3-yl)naphthalen-1-yl]urea |
| 128 | 1-[4-tert-butyl-biphenyl-2-yl]-3-[4-(6-morpholin-4-yl-methyl- |
| | pyridin-3-yl)naphthalen-1-yl]urea |
| 129 | 1-[5-isopropyl-2-methyl-phenyl]-3-[4-(6-morpholin-4-yl-methyl- |
| | pyridin-3-yl)naphthalen-1-yl]urea |
| 130 | 1-[5-sec-butyl-2-methoxy-phenyl]-3-[4-(6-(morpholin-4-yl-methyl- |
| | pyridin-3-yl)naphthalen-1-yl]urea |
| 131 | 1-[5-tert-butyl-2-methoxymethyl-phenyl]3-[4-(6-(morpholin-4-yl- |
| | methyl-pyridin-3-yl)naphthalen-1-yl]urea |
| 132 | 1-[5-tert-butyl-2-methoxy-phenyl]-3-[4-(6-(morpholin-4-yl-methyl- |
| | pyridin-3-yl)naphthalen-1-yl]urea |
| 133 | 1-[5-tert-butyl-2-mewthyl-phenyl]-3-{6-[(3-(methoxy-propyl)- |
| | methyl-amino]-pyridin-3-yl}naphthalen-1-yl-urea |
| 134 | 1-[5-tert-butyl-2-methyl-phenyl]-3-[4-(6-morpholin-4-yl-methyl- |
| | pyridin-3-yl)naphthalen-1-yl]urea |
| 135 | 1-[5-tert-butyl-2-methyl-pyridin-3-yl]-3-[4-(6-morpholin-4-yl- |
| | methyl-pyridin-3-yl)naphthalen-1-yl]urea |
| 136 | 1-[5-(1,1-dimethylpropyl)-2-methoxy-phenyl]-3-[4-(6-morpholin-4- |
| | yl-methyl-pyridin-3-yl)naphthalen-1-yl]urea |
| 137 | 1-[5-tert-butyl-2-(1H-pyrazol-4-yl)-phenyl]-3-[4-(6-morpholin-4-yl- |
| | methyl-pyridin-3-yl)naphthalen-1-yl]urea |
| 138 | 1-[5-tert-butyl-2-(2-methyl-pyrimidin-5-yl)-phenyl]-3-[4-(6- |
| | morpholin-4-yl-methyl-pyridin-3-yl)naphthalen-1-yl]urea |
| 139 | 1-[5-tert-butyl-2-(3-hydroxy-propyl)-phenyl]-3-[4-(6-morpholin- |
| | 4-yl-methyl-pyridin-3-yl)naphthalen-1-yl]urea |
| 140 | 1-[5-tert-butyl-2-(morpholine-4-carbonyl)-phenyl]-3-[4-(6- |
| | morpholin-4-yl-methyl-pyridin-3-yl)naphthalen-1-yl]urea |
| 141 | 1-[5-tert-butyl-2-methoxy-3-{3-[4-(6-morpholin-4-ylmethyl- |
| | pyridin-3-yl)-naphthalen-1-yl]-ureido}-phenyl)-acetamide |
| 142 | 1-[3-methyl-naphthalen-2-yl]-3-[4-(6-morpholin-4-yl-methyl- |
| | pyridin-3-yl)naphthalen-1-yl]urea |
| 143 | 1-[5-tert-butyl-2-methoxy-3-{3-[4-(6-morpholin-4-ylmethyl- |
| | pyridin-3-yl)-naphthalen-1-yl]-urido}-phenyl)-acetamide |
| 144 | 1-[5-tert-butyl-3-(2,3-dihydroxy-propyl)-2-hydroxy- |
| | phenyl]-3-[4-(6-morpholin-4-yl-methyl-pyridin-3- |
| | yl)naphthalen-1-yl]urea |
| 145 | 1-[2,3-dimethyl-1H-indol-5-yl]-3-[4-(6-morpholin-4-yl-methyl- |
| | pyridin-3-yl)naphthalen-1-yl]urea |
| 146 | 1-{5-tert-butyl-2-methyl-3-[3-(tetrahydro-pyran-2-yloxy)-prop-1- |
| | ynyl]-phenyl}-3-[4-(6-morpholin-4-yl-methyl-pyridin-3- |
| | yl)naphthalen-1-yl]urea |
| 147 | 1-[2-methoxy-5-trifluoromethyl-phenyl]-3-[4-(6-morpholin-4-yl- |
| | methyl-pyrimidin-3-yl)naphthalen-1-yl]urea |
| 148 | 1-[5-(2,2-dimethyl-propionyl)-2-methyl-phenyl]-3-[4-(6-morpholin- |
| | 4-yl-methyl-pyridin-3-yl)naphthalen-1-yl]urea |
| 149 | 1-[5-tert-butyl-3-(3-hydroxy-prop-1-ynyl)-2-methyl- |
| | phenyl]-3-[4-(6-morpholin-4-yl-methyl-pyridin-3- |
| | yl)naphthalen-1-yl]urea |
| 150 | 1-[5-tert-butyl-2-(3-hydroxy-propyn-1-ynyl)-phenyl]-3-[4-(6- |
| | morpholin-4-yl-methyl-pyridin-3-yl)naphthalen-1-yl]urea |
| 151 | 1-[5-tert-butyl-3-(2,2-dimethyl-[1,3]dioxolan-4-ylmethyl)-2- |
| | methoxy-phenyl]-3-[4-(6-morpholin-4-yl-methyl-pyridin-3- |
| | yl)naphthalen-1-yl]urea |
| 152 | 1-[5-tert-butyl-3-(2,3-dihydroxy-propyl)-2-methoxy- |
| | phenyl]-3-[4-(6-morpholin-4-yl-methyl-pyridin-3- |
| | yl)naphthalen-1-yl]urea |
| 153 | 1-[5-tert-butyoxy-2-methoxy-phenyl]-3-[4-(6-morpholin-4-yl- |
| | methyl-pyridin-3-yl)naphthalen-1-yl]urea |
| 154 | 1-[5-(1-cyano-cyclopropyl)-2-methoxy-phenyl]-3-[4- |
| | (6-morpholin-4-yl-methyl-pyridin-3-yl)naphthalen-1-yl]urea |
| 155 | 1-[5-tert-butyl-3-(2-diethylamino-ethyl)-2-methoxy- |
| | phenyl]-3-[4-(6-morpholin-4-yl-methyl-pyridin-3- |
| | yl)naphthalen-1-yl]urea |
| 156 | 1-[5-tert-butyl-2-methoxy-phenyl]-3-[4-(6-[1,3]dioxolan-2-yl- |
| | pyridin-3-yl)naphthalen-1-yl]urea |
| 157 | 1-[5-tert-butyl-2-pyrrolidin-1-yl-phenyl]-3-[4-(6-morpholin-4-yl- |
| | methyl-pyridin-3-yl)naphthalen-1-yl]urea |
| 158 | 1-[5-tert-butyl-2-dimethylamino-phenyl]-3-[4-(6-morpholin-4-yl- |
| | methyl-pyridin-3-yl)naphthalen-1-yl]urea |
| 159 | 1-[5-tert-butyl-2-propoxy-phenyl]-3-[4-(6-morpholin-4-yl-methyl- |
| | pyridin-3-yl)naphthalen-1-yl]urea |
| 160 | 1-[5-tert-butyl-2-methoxy-phenyl]-3-[4-(6-hydroxymethyl- |
| | pyridin-3-yl)naphthalen-1-yl]urea |
| 161 | 1-[5-tert-butyl-2-methoxy-phenyl]-3-{4-[6-(2,6-dimethyl- |
| | morpholin-4-yl-methyl)pyridin-3-yl]naphthalen-1-yl}urea |
| 162 | 1-[5-cyclohexyl-2-methoxy-phenyl]-3-[4-(6-morpholin-4-yl- |
| | methyl-pyridin-3-yl)naphthalen-1-yl]urea |
| 163 | 1-[2,4-dimethoxy-5-trifluoromethyl-phenyl]-3-[4-(6- |
| | morpholin-4-yl-methyl-pyridin-3-yl)naphthalen-1-yl]urea |
| 164 | 1-[5-tert-butyl-2-methoxy-3-nitro-phenyl]-3-[4-(6-morpholin-4-yl- |
| | methyl-pyridin-3-yl)naphthalen-1-yl]urea |
| 165 | 1-[3-amino-5-tert-butyl-2-methoxy-phenyl]-3-[4-(6-methyl- |
| | pyridin-3-yl)naphthalen-1-yl]urea |
| 166 | N-acetyl-N-(5-tert-butyl-2-methoxy-3-{3-[4-(6-morpholin-4- |
| | ylmethyl-pyridin-3-yl)-naphthalen-1-yl]-ureido}-phenyl)-acetamide |
| 167 | 1-[6-tert-butyl-4-methyl-3-oxo-3,4-dihydro-2H- |
| | benzo[1,4]oxazin-8-yl]-3-[4-(6-morpholin-4-ylmethyl- |
| | pyridin-3-yl)naphthalen-1-yl]urea |
| 168 | 1-[5-tert-butyl-2-ethoxy-phenyl]-3-[4-(6-morpholin-4-yl-methyl- |
| | pyridin-3-yl)naphthalen-1-yl]urea |
| 169 | 1-[5-tert-butyl-2-isopropoxy-phenyl]-3-[4-(6-morpholin-4-yl- |
| | methyl-pyridin-3-yl)naphthalen-1-yl]urea |
| 170 | 1-[5-tert-butyl-2-imidazol-1-yl-phenyl]-3-[4-(6-morpholin-4-yl- |
| | methyl-pyridin-3-yl)naphthalen-1-yl]urea |
| 171 | 1-[5-tert-butyl-3-ethylamino-2-methoxy-phenyl]-3-[4-(6- |
| | morpholin-4-yl-methyl-pyridin-3-yl)naphthalen-1-yl]urea |
| 172 | 1-[5-tert-butyl-2-methoxy-3-{3-[4-(6-morpholin-4-ylmethyl- |
| | pyridin-3-yl)-naphthalen-1-yl]-ureido}-phenyl)- |
| | bis(methanesulfon)amide |
| 173 | 1-[5-tert-butyl-2-(1-methyl-1H-pyrazol-4-yl)-phenyl]-3-[4-(6- |
| | morpholin-4-yl-methyl-pyridin-3-yl)naphthalen-1-yl]urea |
| 174 | 1-[2-methanesulfinyl-5-trifluoromethyl-phenyl]-3-[4-(6- |
| | morpholin-4-yl-methyl-pyridin-3-yl)naphthalen-1-yl]urea |
| 175 | 1-[4-(6-{[bis(2-methoxy-ethyl)-amino]-methyl}-pyridin-3-yl- |
| | naphthalen-1-yl]-3-[5-tert-2-methoxy-phenyl]urea |
| 176 | N-[1-(5-{4-[3-(5-tert-butyl-2-methoxy-phenyl)-ureido]- |
| | naphthalen-1-yl}-pyridin-2-ylmethyl)-pyrrolidin-3- |
| | yl]-acetamide |
| 177 | 1-[1-acetyl-3,3-dimethyl-2,3-dihydro-1H-indol-5-yl]-3-[4-(6- |
| | morpholin-4-yl-methyl-pyridin-3-yl)naphthalen-1-yl]urea |
| 178 | 1-(5-tert-butyl-2-methoxy-3-{3-[4-(6-morpholin-4-ylmethyl- |
| | pyridin-3-yl)-naphthalen-1-yl]-ureido}-phenyl)- |
| | propionamide |
| 179 | 1-[5-tert-butyl-2-methyl-benzooxazol-7-yl]-3-[4-(6-morpholin-4-yl- |
| | methyl-pyridin-3-yl)naphthalen-1-yl]urea |
| 180 | 1-[3-trifluoromethanesulfonyl-phenyl]-3-[4-(6-morpholin-4-yl- |
| | methyl-pyridin-3-yl)naphthalen-1-yl]urea |
| 181 | N-(5-tert-butyl-2-methoxy-3-{3-{4-(6-morpholin-4-ylmethyl- |
| | pyridin-3-yl)-naphthalen-1-yl]-ureidol}-phenyl)- |
| | isobutyramide |
| 182 | 2-(4-tert-butyl-2-{3-[4-(6-morpholin-4-ylmethyl-pyridin-3-yl)- |
| | naphthalen-1-yl]-ureido}-phenoxy)-acetamide |
| 183 | 1-[5-tert-butyl-2-oxo-2,3-dihydro-benzooxazol-7-yl]-3-[4-(6- |
| | morpholin-4-yl-methyl-pyridin-3-yl)naphthalen-1-yl]urea |
| 184 | 1-[5-tert-butyl-3-cyano-2-methoxy-phenyl]-3-[4-(6-morpholin-4-yl- |
| | methyl-pyridin-3-yl)naphthalen-1-yl]urea |
| 185 | 1-[5-tert-butyl-benzooxazol-7-yl]-3-[4-(6-morpholin-4-yl-methyl- |
| | pyridin-3-yl)naphthalen-1-yl]urea |
| 186 | 5-tert-butyl-2-methoxy-{3-[4-(6-morpholin-4-ylmethyl- |
| | pyridin-3-yl)-naphthalen-1-yl]-ureido}-phenyl)- |
| | benzenesulfonamide |
| 187 | Ethanesulfonic acid (5-tert-butyl-2-methoxy-3-{3-[4-(6- |
| | morpholin-4-ylmethyl-pyridin-3-yl)-naphthalen-l- |
| | yl]-ure3ido}-phenyl)-amide |
| 188 | 1-[5-tert-butyl-2-methoxy-phenyl]-3-[4-(2-morpholin-4-yl-methyl- |
| | pyrimidin-5-yl)naphthalen-1-yl]urea |
| 189 | 1-[5-tert-butyl-2-methylsulfanyl-phenyl]-3-[4-(6-morpholin-4-yl- |
| | methylpyridin-3-yl)naphthalen-1-yl]urea |
| 190 | 1-[5-tert-butyl-2-methoxy-pyridin-3-yl]-3-[4-(6-morpholin-4-yl- |
| | methyl6-pyridin-3-yl)naphthalen-1-yl]urea |
| 191 | 2,2,2-trifluoroethanesulfonic acid (5-tert-butyl-2-methoxy- |
| | 3-{3-[4-(6-morpholin-4-ylmethyl-pyridin-3-yl)-naphthalen- |
| | 1-yl]-ureido}-phenyl)-amide |
| 192 | N-(5-{4-[3-(5-tert-butyl-2-methyl-phenyl)-ureido]-naphthalen- |
| | 1-yl}-pyrazin-2-yl)-methanesulfonamide |
| 193 | 1-[5-tert-butyl-2-methoxy-phenyl]-3-[4-(6-{[bis-(2-cyano-ethyl)- |
| | amino]-methyl}-pyridin-3-yl)naphthalen-1-yl]urea |
| 194 | 1-[5-tert-butyl-2-methoxy-phenyl]-3-{4-[6-(4-methyl-piperazin-1- |
| | ylmethyl)pyridin-3-yl]naphthalen-1-yl}urea |
| 195 | 1-[5-tert-butyl-2-methoxy-phenyl]-3-[4-(6-thiomorpholin-4-yl- |
| | methyl-pyridin-3-yl)naphthalen-1-yl]urea |
| 196 | 1-[5-tert-butyl-2-methoxy-phenyl]-3-{4-[6-(2,6-dimethyl- |
| | piperidin-1-ylmethyl)-pyridin-3-yl]naphthalen-1-yl}urea |
| 197 | 1-[5-tert-butyl-2-methoxy-phenyl]-3-{4-[6-(1-oxo- |
| | tetrahydropyran-4-ylamino)-pyridin-3-yl]naphthalen- |
| | 1-yl}urea |
| 198 | 1-[5-tert-butyl-2-methoxy-phenyl]-3-{4-[6-(tetrahydropyran-4- |
| | ylamino)-pyridin-3-yl]naphthalen-1-yl}urea |
| 199 | 1-[5-tert-butyl-2-methoxy-phenyl]-3-[4-[6-{[(2-cyano-ethyl)- |
| | tetrahydro-furan-2-ylmethyl)-amino]-methyl}-pyridin-3- |
| | yl)naphthalen-1-yl}urea |
| 200 | 1-[5-tert-butyl-2-methoxy-phenyl]-3-{4-[6-(2-methoxymethyl- |
| | morpholin-4-ylmethyl)-pyridin-3-yl]naphthalen-1-yl}urea |
| 201 | 1-[5-tert-butyl-2-methoxy-phenyl]-3-{4-[6-(2-methyl-3-oxo- |
| | piperazin-1-ylmethyl)-pyridin-3-yl]naphthalen-1-yl}urea |
| 202 | 1-(5-{4-[3-(5-tert-butyl-2-methoxy-phenyl)-ureido]- |
| | naphthalen-1-yl}-pyridin-2-ylmethyl)-piperidine-3- |
| | carboxylic acid amide |
| 203 | 1-[5-tert-butyl-2-methoxy-phenyl]-3-{4-[6-(1-oxo-thiomorpholin-4- |
| | ylmethyl)-pyridin-3-yl]naphthalen-1-yl}urea |
| 204 | 1-(3,3-dimethyl-2-oxo-2,3-dihydro-1H-indol-5-yl)-3-[4-(6- |
| | morpholin-4-ylmethyl-pyridin-3-yl)-naphthalen-1-yl]urea |
| 205 | 1-[5-tert-butyl-2-methoxy-phenyl]-3-{4-[6-(3-oxo-piperazin-1- |
| | ylmethyl)-pyridin-3-yl]naphthalen-1-yl}urea |
| 206 | 1-[5-tert-butyl-2-methoxy-phenyl]-3-(4-{6-[(tetrahydro-furan-3- |
| | ylamino)methyl]-pyridin-3-yl}naphthalen-1-yl}urea |
| 207 | 1-[5-tert-butyl-2-methoxy-phenyl]-3-[4-(6-{[(2-cyano-ethyl)- |
| | pyridin-3-ylmethyl-amino]-methyl}-pyridin-3-yl)naphthalen- |
| | 1-yl]urea |
| 208 | 1-[5-tert-butyl-2-methoxy-phenyl]-3-{4-[6-(2-oxa-5-aza- |
| | bicyclo[2.2.1]hept-5-ylmethyl)-pyridin-3-yl]naphthalen-1-yl}urea |
| 209 | 1-[5-tert-butyl-2-methoxy-phenyl]-3-{4-[6-(2,6-dimethyl- |
| | morpholin-4-ylmethyl)pyridin-3-yl]naphthalen-1-yl}urea |
| 210 | 1-[5-tert-butyl-2-methoxy-phenyl]-3-(4-{6-[4-(3-methoxy- |
| | phenyl)-1-piperazin-1-ylmethyl]-pyridin-3-yl}naphthalen- |
| | 1-yl)urea |
| 211 | 1-[5-tert-butyl-2-methoxy-phenyl]-3-{4-[6-(morpholine-4- |
| | carbonyl)-pyridin-3-yl]naphthalen-1-yl}urea |
| 212 | 1-[5-tert-butyl-2-methoxy-phenyl]-3-[4-(5-morpholin-4-ylmethyl- |
| | pyrazin-2-yl)-naphthalen-1-yl}urea |
| 213 | 1-[6-tert-butyl-3-oxo-3,4-dihydro-2H-benzo[1,4]oxazin-8- |
| | yl]-3-[4-(6-morpholin-4-ylmethyl-pyridin-3-yl) naphthalen- |
| | 1-yl]urea |
| 214 | 1-[3-amino-5-tert-butyl-2-methoxy-phenyl]-3-[4-(6-morpholin-4-yl- |
| | methyl-pyridin-3-yl)naphthalen-1-yl]urea |
| 215 | N-(5-{4-[3-(5-tert-butyl-2-methoxy-phenyl)-ureido]-naphthalen-1- |
| | yl}pyridin-2-yl)-acetamide |
| 216 | N-(5-tert-butyl-2-methoxy-3-{3-[4-(6-morpholin-4-ylmethyl- |
| | pyridin-3-yl)-naphthalen-1-yl]-ureido}-phenyl)-N-methyl- |
| | acetamide |
| 217 | N-(5-tert-butyl-2-methoxy-3-{3-[4-(6-morpholin-4-ylmethyl- |
| | pyridin-3-yl)-naphthalen-1-yl]-ureido}-phenyl)- |
| | 2,2,2-trifluoro-acetamide |
| 218 | 1-[5-tert-butyl-2-methoxy-phenyl]-3-{4-[6-(pyridin-3-yloxy)- |
| | pyridin-3-yl]naphthalen-1-yl}urea |
| 219 | [4-(6-morpholin-4-ylmethyl-pyridin-3-yl)-naphthalen-1-yl]- |
| | carbamic acid 3-tert-butyl-phenyl ester |
| 220 | N-(5-tert-butyl-2-methoxy-3-{3-[4-(6-morpholin-4-ylmethyl- |
| | pyridin-3-yl)-naphthalen-1-yl]-ureido}-phenyl)- |
| | methanesulfonamide |
| 221 | 1-[2-carbomethoxy-5-tert-butyl-3-thienyl]-3-[4-(2-morpholin-4-yl- |
| | ethoxy)naphthalen-1-yl]urea |
| 222 | 1-[2-methylcarbamoyl-5-tert-butyl-3-thienyl]-3-[4-(2- |
| | morpholin-4-yl-ethoxy)naphthalen-1-yl]urea |
| 223 | 1-[2-methylcarbamoyl-5-tert-butyl-3-thienyl]-3-[4-(2-(trans-2,6- |
| | dimethylmorpholin-4-yl)ethoxy)naphthalen-1-yl]urea |
| 224 | 1-[2-methylcarbamoyl-5-tert-butyl-3-thienyl]-3-[4-(2-(2- |
| | dimethylaminomethyldimethylmorpholin- |
| | 4-yl)ethoxy)naphthalen-1-yl]urea |
| 225 | 1-[2-carbomethoxy-5-tert-butyl-3-thienyl]-3-[4-(2-(1- |
| | oxothiomorpholin-4-yl)ethoxy)naphthalen-1-yl]urea |
| 226 | 1-[2-methylcarbamoyl-5-tert-butyl-3-thienyl]-3-[4-(2-(1- |
| | oxothiomorpholin-4-yl)ethoxy)naphthalen-1-yl]urea |
| 227 | 1-[3-carbomethoxy-5-tert-butyl-2-thienyl]-3-[4-(2-(1- |
| | oxothiomorpholin-4-yl)ethoxy)naphthalen-1-yl]urea |
| 228 | 1-[2-carbomethoxy-5-tert-butyl-3-thienyl]-3-[4-(2- |
| | (tetrahydropyran-4-yl)ethoxy)naphthalen-1-yl]urea |
| 229 | 1-[2-methylcarbamoyl-5-tert-butyl-3-thienyl]-3-[4-(2- |
| | (tetrahydropyran-4-yl)ethoxy)naphthalen-1-yl]urea |
| 230 | 1-[3-carbomethoxy-5-tert-butyl-2-thienyl]-3-[4-(2- |
| | (tetrahydropyran-4-yl)ethoxy)naphthalen-1-yl]urea |
| 231 | 1-[2-carbomethoxy-5-tert-butyl-3-thienyl]-3-[4-(2-(1-oxo- |
| | tetrahydrothiophen-3-yl)ethoxy)naphthalen-1-yl]urea |
| 232 | 1-[2-methylcarbamoyl-5-tert-butyl-3-thienyl]-3-[4-(2-(1-oxo- |
| | tetrahydrothiophen-3-yl)ethoxy)naphthalen-1-yl]urea |
| 233 | 1-[2-carbomethoxy-5-tert-butyl-3-thienyl]-3-[4-(pyridin-4-yl- |
| | methoxy)naphthalen-1-yl]urea |
| 234 | 1-[2-methylcarbamoyl-5-tert-butyl-3-thienyl]-3-[4-(pyridin-4- |
| | yl)methoxy)naphthalen-1-yl]urea |
| 235 | 1-[2-carbomethoxy-5-tert-butyl-3-thienyl]-3-[4-(2-imidazol-1-yl- |
| | ethoxy)naphthalen-1-yl]urea |
| 236 | 1-[2-carbomethoxy-5-tert-butyl-3-thienyl]-3-[4-(2-(3,4- |
| | dimethoxyphenyl)-ethoxy)naphthalen-1-yl]urea |
| 237 | 1-[2-methylcarbamoyl-5-tert-butyl-3-thienyl]-3-[4-(2-(3,4- |
| | dimethoxyphenyl)ethoxy)naphthalen-1-yl]urea |
| 238 | 1-[2-carbomethoxy-5-tert-butyl-3-pyrrolyl]-3-[4-(2-morpholin-4-yl- |
| | ethoxy)naphthalen-1-yl]urea |
| 239 | 1-[2-methylcarbamoyl-5-tert-butyl-3-pyrrolyl]-3-[4-(2- |
| | morpholin-4-yl-ethoxy)naphthalen-1-yl]urea |
| 240 | 1-[1-methyl-2-carbomethoxy-5-tert-butyl-3-pyrrolyl]-3-[4-(2- |
| | morpholin-4-yl-ethoxy)naphthalen-1-yl]urea |
| 241 | 1-[2-carbomethoxy-5-tert-butyl-3-pyrrolyl]-3-[4-(2-(1- |
| | oxothiomorpholin-4-yl)ethoxy)naphthalen-1-yl]urea |
| 242 | 1-[2-methylcarbamoyl-5-tert-butyl-3-pyrrolyl]-3-[4-(2-(1- |
| | oxothiomorpholin-4-yl)ethoxy)naphthalen-1-yl]urea |
| 243 | 1-[1-methyl-2-carbomethoxy-5-tert-butyl-3-Pyrroly]-3-[4-(2-(1- |
| | oxothiomorpholin-4-yl)ethoxy)naphthalen-1-yl]urea |
| 244 | 1-[2-carbomethoxy-5-tert-butyl-3-pyrrolyl]-3-[4-(2- |
| | (tetrahydropyran-4-yl)ethoxy)naphthalen-1-yl]urea |
| 245 | 1-[2-methylcarbamoyl-5-tert-butyl-3-pyrrolyl]-3-[4-(2- |
| | (tetrahydropyran-4-yl)ethoxy)naphthalen-1-yl]urea |
| 246 | 1-[2-carbomethoxy-5-tert-butyl-3-pyrrolyl]-3-[4-(2-(1-oxo- |
| | tetrahydrothiophen-3-yl)ethoxy)naphthalen-1-yl]urea |
| 247 | 1-[2-methylcarbamoyl-5-tert-butyl-3-pyrrolyl]-3-[4-(2-(1-oxo- |
| | tetrahydrothiophen-3-yl)ethoxy)naphthalen-1-yl]urea |
| 248 | 1-[1-methyl-2-carbomethoxy-5-tert-butyl-3-pyrrolyl]-3-[4-(2-(1- |
| | tetrahydrothiophen-3-yl)ethoxy)naphthalen-1-yl]urea |
| 249 | 1-[2-carbomethoxy-5-tert-butyl-3-pyrrolyl]-3-[4-(pyridin-4-yl- |
| | methoxy)naphthalen-1-yl]urea |
| 250 | 1-[2-methylcarbamoyl-5-tert-butyl-3-pyrrolyl]-3-[4-pyridin-4-yl- |
| | methoxy)naphthalen-1-yl]urea |
| 251 | 1-[2-carbomethoxy-5-tert-butyl-3-pyrrolyl]-3-[4-(pyridin-4-yl- |
| | ethoxy)naphthalen-1-yl]urea |
| 252 | 1-[2-carbomethoxy-5-tert-butyl-3-pyrrolyl]-3-[4-(2-imidazol-1-yl- |
| | ethoxy)naphthalen-1-yl]urea |
| 253 | 1-[2-carbomethoxy-5-tert-butyl-3-pyrrolyl]-3-[4-(2-(3,4- |
| | dimethoxyphenyl)ethoxy)naphthalen-1-yl]urea |
| 254 | 1-[2-methylcarbamoyl-5-tert-butyl-3-pyrrolyl]-3-[4-(2-(3,4- |
| | dimethoxyphenyl)ethoxy)naphthalen-1-yl]urea |
| 255 | 1-(5-tert-butyl-2-methoxyphenyl)-3-[4-(2-methoxy-pyridin- |
| | 4-yl-oxy)-naphthalen-1-yl]urea |
| 256 | 1-(5-tert-butyl-2-methoxyphenyl)-3-[4-(2-methyl-pyridin-4-yl-oxy)- |
| | naphthalen-1-yl]urea |
| 257 | 1-(5-tert-butyl-2,3-dimethoxyphenyl)-3-[4-(2-methoxy-pyridin- |
| | 4-yl-oxy)-naphthalen-1-yl]urea |
| 258 | 5-tert-butyl-2-methoxy-3-{3-(pyridin-4-yl-oxy)naphthalen-1- |
| | yl]ureido}benzamide |
| 259 | Morpholine carboxylic acid (5-tert-butyl-2-methoxy-3-{3-[- |
| | (pyridin-4-yloxy)naphthalen-1-yl}ureido}phenyl)amide |
| 260 | N-(5-tert-butyl-2-methoxy-3-{3-[-(pyridin4-yl-oxy)naphthalen-1- |
| | yl]ureido}phenyl)acetamide |
| 261 | 3-(5-tert-butyl-2-methoxy-3-{3-[-(pyridin-4-yl-oxy)naphthalen-1- |
| | yl]ureido}phenyl)-1,1-dimethylurea |
| 262 | 1-(5-tert-butyl-2,3-dimethoxy-phenyl)-3-[4-(2-amino-pyridin-4- |
| | yloxy)-naphthalen-1-yl]urea |
| 263 | 1-(5-tert-butyl-2,3-dimethoxyphenyl)-3-[4-(2-methylamino- |
| | pyridin-4-yloxy]-naphthalen-1-yl}urea |
| 264 | 1-(5-tert-butyl-2,3-dimethoxyphenyl)-3-{4-[2-(1-phenyl- |
| | ethylamino)-pyridin-4-yloxy]-naphthalen-1-yl}urea |
| 265 | 1-(5-tert-butyl-2-methoxy-pyridin-3-yl)-3-[4-(2-amino-pyridin-4- |
| | yloxy)-naphthalen-1-yl]urea |
| 266 | 1-(5-tert-butyl-2-methoxy-pyridin-3-yl)-3-[4-(2-methylamino- |
| | pyridin-4-yloxy]-naphthalen-1-yl}urea |
| 267 | 1-(5-tert-butyl-2-methoxy-pyridin-3-yl)-3-{4-[2-(1-phenyl- |
| | ethylamino)-pyridin-4-yloxy]-naphthalen-1-yl}urea |
| 268 | 1-(5-tert-butyl-2-methyl-phenyl)-3-[4-(2-aminopyridin-4-yl-oxy)- |
| | naphthalen-1-yl]urea |
| 269 | 1-(5-tert-butyl-2-morpholin-4-yl-phenyl)-3-[4-(2-morpholin-4-yl- |
| | ethoxy)-naphthalen-1-yl]urea |
| 270 | 1-(5-tert-butyl-2-methoxyphenyl)-3-[4-(2-methylpyridin-4-yl-oxy)- |
| | naphthalen-1-yl]urea |
| 271 | 1-(5-tert-butyl-2,3-dimethoxyphenyl)-3-[4-(2-methoxypyridin-4-yl- |
| | oxy)-naphthalen-1-yl]urea |
| 272 | 5-tert-butyl-2-methoxy-3-{3-[-(pyridin-4-yl-oxy)naphthalen-1- |
| | yl]ureido}benzamide |
| 273 | Morpholine-4-carboxylic acid (5-tert-butyl-2-methoxy-3-{3-[- |
| | (pyridin-4-yl-oxy)naphthalen-1-yl]ureido}phenyl)amide |
| 274 | N-(5-tert-butyl-2-methoxy-3-{3-[-(pyridin-4-yl-oxy)naphthalen-1- |
| | yl]}ureido}phenyl)acetamide |
| 275 | 3-(5-tert-butyl-2-methoxy-3-{3-[-(pyridin-4-yl-oxy)naphthalen-1- |
| | yl]ureido}phenyl)-1,1-dimethylurea |
| 276 | 1-(5-tert-butyl-2,3-dimethoxy-phenyl)-3-[4-(2-amino-pyridin-4- |
| | yloxy)-naphyhalen-1-yl]urea |
| 277 | 1-(5-tert-butyl-2,3-dimethoxy-phenyl)-3-[4-(2-methylamino- |
| | pyridin-4-yloxy)-naphthalen-1-yl]urea |
| 278 | 1-(5-tert-butyl-2,3-dimethoxy-phenyl)-3-[4-(2-phenyl-ethylamino- |
| | pyridin-4-yloxy)-naphthalen-1-yl]urea |
| 279 | 1-(5-tert-butyl-2-methoxy-pyridin-3-yl)-3-[4-(2-amino-pyridin-4- |
| | yloxy)-naphthalen-1-yl]urea |
| 280 | 1-(5-tert-butyl-2-methoxy-pyridin-3-yl)-3-[4-(2-methylamino- |
| | pyridin-4-yloxy)-naphthalen-1-yl]urea |
| 281 | 1-(5-tert-butyl-2-methoxy-pyridin-3-yl)-3-{4-[2-(1-phenyl- |
| | ethylamino)-pyridin-4-yloxy)-naphthalen-1-yl]urea |
| 282 | 1-(5-tert-butyl-2-methyl-phenyl)-3-[4-(2-aminopyridin-4-yloxy)- |
| | naphthalen-1-yl]urea |
| 283 | 1-(5-tert-butyl-2-morpholin-4-yl-phenyl)-3-[4-(2-morpholin-4-yl- |
| | ethoxy)-naphthalen-1-yl]urea |

One of ordinary skill in the art will recognize that some of the compounds of this invention can exist in different geometrical isomeric forms. A number of the compounds of this invention possess asymmetric carbons and can therefore exist in racemic and optically active forms as well as in the form of racemic or nonracemic mixtures thereof, and in the form of diastereomers and diastereomeric mixtures. All of these compounds, including cis isomers, trans isomers, diastereomic mixtures, racemates, nonracemic mixtures of enantiomers, substantially pure, and pure enantiomers, are considered to be within the scope of the present invention and are collectively referred to when reference is made to compounds of this invention.

Methods of separation of enantiomeric and diastereomeric mixtures are well known to one skilled in the art. The optical isomers can be obtained by resolution of the racemic mixtures according to conventional processes, for example, by the formation of diastereoisomeric salts using an optically active acid or base. Examples of appropriate acids are tartaric, diacetyltartaric, dibenzoyltartaric, ditoluoyltartaric and camphorsulfonic acid. Mixtures of diastercoisomers can be separated into their individual diastereomers on the basis of their physical chemical differences by methods known to those skilled in the art, for example, by chromatography or fractional crystallization. The optically active bases or acids are liberated from the separated diastereomeric salts.

Another process for separation of optical isomers involves the use of a chiral chromatography column (e.g., chiral HPLC columns) optimally chosen to maximize the separation of the enantiomers. Suitable chiral HPLC columns are manufactured by Diacel, e.g., Chiracel OD and Chiracel OJ. The optically active compounds of this invention can likewise be obtained by utilizing optically active starting materials.

The present invention encompasses the administration of any isolated racemic or optically active form of compounds of this invention which possess raf kinase inhibitory activity angiogenesis inhibitory activity. The present invention also encompasses the administration of any isolated racemic or optically active form of compounds of this invention which possess angiogenesis inhibitory activity.

The term stereoisomer is understood to encompass diastereoisomers, enantiomers, geometric isomers, etc. Herein, substantially pure enantiomers is intended to mean that no more than 5% w/w of the corresponding opposite enantiomer is present.

Pharmaceutically acceptable salts of these compounds as well as commonly used prodrugs of these compounds are also within the scope of the invention.

Salts of this invention are especially the pharmaceutically acceptable salts of compounds of formula (I) such as, for example, organic or inorganic acid addition salts of compounds of formula (I). Suitable inorganic acids include but are not limited to halogen acids (such as hydrochloric acid and hydrobromic acid), sulfuric acid, or phosphoric acid. Suitable organic acids include but are not limited to carboxylic, phosphonic, sulfonic, or sulfamic acids, with examples including acetic acid, propionic acid, octanoic acid, decanoic acid, trifluoroacetic acid, dodecanoic acid, glycolic acid, lactic acid, 2- or 3-hydroxybutyric acid, γ-aminobutyric acid (GABA), gluconic acid, glucosemonocarboxylic acid, benzoic acid, salicylic acid, phenylacetic acid and mandelic acid, fumaric acid, succinic acid, adipic acid, pimelic acid, suberic acid, azeiaic acid, malic acid, tartaric acid, citric acid, glucaric acid, galactaric acid, amino acids (such as glutamic acid, aspartic acid, N-methylglycine, acetytaminoacetic acid, N-acetylasparagine or N-acetylcysteine), pyruvic acid, acetoacetic acid, methanesulfonic acid, tri-fluoromethanesulfonic acid, 4-toluenesulfonic acid, benzenesulfonic acid, 1-naphthalenesulfonic acid, 2-naphthalenesulfonic acid, phosphoserine, and 2- or 3-glycerophosphoric acid.

In addition, pharmaceutically acceptable salts include acid salts of inorganic bases, such as salts containing alkali metal cations (e.g., Li⁺ Na⁺ or K⁺), alkaline earth metal cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N,N-diethylamine, N,N-dicyclohexylamine, lysine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU).

The formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., published by Williams & Wilkins, pages 27-29, (1995) which is hereby incorporated by reference). Commonly used prodrugs are designed to take advantage of the major drug biotransformation reactions and are also to be considered within the scope of the invention. Major drug biotransformation reactions include N-dealkylation, o-dealkylation, aliphatic hydroxylation, aromatic hydroxylation, N-oxidation, S-oxidation, deamination, hydrolysis reactions, glucuronidation, sulfation and acetytation (see Goodman and Gilman's The Pharmacological Basis of Therapeutics (Ninth Edition), editor Molinoff et al., publ. by McGraw-Hill, pages 11-13, (1996), which is hereby incorporated by reference).

The invention also relates to methods for treating and preventing diseases, for example, angiogenesis disorders in humans and/or other mammals by administering a compound of this invention or a pharmaceutical composition comprising one or more compounds of this invention. An embodiment of this invention is a method for treating and preventing angiogenesis disorders in humans and/or other mammals by administering a compound of this invention or a pharmaceutical composition comprising one or more compounds of this invention to a human or other mammal.

The invention also relates to methods for treating and preventing hyper-proliferative disorders such as cancer and inflammatory disorders by administering a compound of this invention or a pharmaceutical composition thereof. An embodiment of this invention is a method for treating and preventing hyper-proliferative disorders such as cancer and inflammatory disorders in humans and/or other mammals by administering a compound of this invention or a pharmaceutical composition comprising one or more compounds of this invention to a human or other mammal.

The invention also relates to a method of treating or preventing cancer and other hyper proliferative disorders by administering a compound of this invention or a pharmaceutical composition comprising one or more compounds of this invention, in combination with a cytotoxic agent. An embodiment of this invention is a method for treating and preventing hyper-proliferative disorders such as cancer in humans and/or other mammals by administering a pharmaceutical composition comprising one or more compounds of this invention and one or more cytotoxic agents to a human or other mammal.

Optional anti-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, predhisolone, prednisone, procarbazine, raloxifen, streptozocin, tamoxifen, thioguanine, topotecan, vinblastine, vincristine, and vindesine.

Additional cytotoxic agents include gemcitabine, taxotere, BCNU, CCNU, DTIC, 5-fluorouarcil, herceptin and actinomycin D. Other anti-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyturidine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, oxaliplatin, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-proliferative agents suitable for use with the composition of the invention include but are not limited to other anti-cancer agents such as epothilone, irinotecan, raloxifen and topotecan.

The present invention provides methods for treating a cancer in a mammal, especially a human patient, comprising administering a compound according to the invention optionally in combination with a cytotoxic or cytostatic chemotherapeutic agent including, but not limited to, DNA topoisomerase I and II inhibitors, DNA intercalators, alkylating agents, microtubule disruptors, hormone and growth factor receptor agonists or antagonists, other kinase inhibitors and antimetabolites.

The methods of the present invention can be used to treat a variety of human cancers, including but not limited to pancreatic, lung, colon, ovarian, prostate, leukemia, melanoma, hepatocellular, renal, head and neck, glioma, and mammary carcinomas.

The compound according to the invention and the cytotoxic or cytostatic agent are administered to a mammal in quantities which together are therapeutically effective against proliferative diseases including but not limited to colon, gastric, lung, pancreatic, ovarian, prostate, leukemia, melanoma, hepatocellular, renal, head and neck, glioma, and mammary cancers. Thus, the compound according to the invention is effective for raf kinase-mediated cancers. However, these compounds are also effective for cancers not mediated by raf kinase.

A compound according to the invention can be administered simultaneously with a cytotoxic or cytostatic agent to a patient with a cancer, in the same formulation or, more typically in separate formulations and, often, using different administration routes. Administration can also be sequentially, in any order.

A compound according to the invention can be administered in tandem with the cytotoxic or cytostatic agent, wherein a compound according to the invention can be administered to a patient once or more per day for up to 28 consecutive days with the concurrent or intermittent administration of a cytotoxic or cytostatic agent over the same total time period.

A compound according to the invention can be administered to a patient at an oral, intravenous, intramuscular, subcutaneous, or parenteral dosage which can range from about 0.1 to about 200 mg/kg of total body weight and the cytotoxic or cytostatic agent can be administered to a patient at an intravenous, intramuscular, subcutaneous, or parenteral dosage which can range from about 0.1 mg to 200 mg/kg of patient body weight.

This invention further relates to kits comprising separate doses of the two mentioned chemotherapeutic agents in separate containers. The combinations of the invention can also be formed in vivo, e.g., in a patient's body.

The term “cytotoxic” refers to an agent which can be administered to kill or eliminate a cancer cell. The term “cytostatic” refers to an agent who can be administered to restrain tumor proliferation rather than induce cytotoxic cytoreduction yielding an elimination of the cancer cell from the total viable cell population of the patient. The chemotherapeutic agents described herein, e.g., irinotecan, vinorelbine, gemcitabine, and paclitaxel are considered cytotoxic agents. These cytotoxic and cytostatic agents have gained wide spread use as chemotherapeutics in the treatment of various cancer types and are well known. Examples are given below.

Irinotecan (CPT-11) is sold under the trade name of Camptosar® by Pharmacia & Upjohn Co., Kalamazoo, Mich. Irinotecan is a camptothecin or topoisomerase I inhibitor.

Vinorelbine (Vinorelbine tartrate) has the molecular formula C₄₅H₅₄N₄O_{8.}2C₄H₆O₆ with a molecular weight of 1079.12 and is sold under the tradename of Navelbine® by Glaxo SmithKline, Research Triangle Park. Vinorelbine is a semi-synthetic vinca alkaloid with antitumor activity. The chemical name is 3′,4′-didehydro-4′deoxy-C-norvincaleukoblastine [R-(R,R)-2,3-dihydroxybutanedioate (1:2)(salt)].

Gemcitabine, a pyrimidine analog, is sold under the trade name Gemzar® (Eli Lilly & Co., Indianapolis, Ind.).

Paclitaxel is sold under the tradename Taxol® by the Bristol-Myers Squibb Company. Paclitaxel, [2aR-[2aα,4β,4aβ,6β,9α(αR*,βS*),11α,12α,12aα12bα]]-β-(Benzoylamino)-α-hydroxybenzenepropanoic acid 6,12b-bis(acetyloxy)-12-(benzoyloxy)-2a,3,4, 4a, 5, 6, 9, 10, 11, 12, 12a, 12b-dodecahydro-4,11-dihydroxy-4a,8, 13,13-tetramethyl-5-oxo-7,11-methano-1H-cyclodeca[3,4]benz-[1,2-b]oxet-9-yl ester, has the empirical formula C₄₇H₅₁NO₁₄ and a molecular weight of 853.9.

These and other cytotoxic/cytostatic agents can be administered in the conventional formulations and regimens in which they are known for use alone.

Description of Treatment of Hyperproliferative Disorders

Cancer and hyperproliferative disorders are defined as follows. These disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer.

Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallblader, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

These disorders have been well characterized in man, but also exist with a similar etiology in other mammals, and can be treated by pharmaceutical compositions of the present invention.

Conditions within a human or other mammal which can be treated by administering a compound of this invention are those characterized by abnormal angiogenesis or hyperpermiability processes. Specific conditions to be treated include tumor growth, retinopathy, including diabetic retinopathy, ischemic retinal-vein occlusion, retinopathy of prematurity and age related macular degeneration; rheumatoid arthritis, psoriasis, or a bullous disorder associated with subepidermal blister formation, including bullous pemphigoid, erythema multiforme, and dermatitis herpetiformis.

An embodiment of the present invention is a method for treating diseases in humans and/or other mammals which are mediated by the VEGF induced signal transduction pathway which comprises administering a compound of this invention or a pharmaceutical composition with one or more compounds of this invention to a human or other mammal.

Another embodiment of this invention is a method for treating diseases in humans and/or other mammals which are characterized by abnormal angiogenesis or hyperpermiability processes which comprises administering a compound of this invention or a pharmaceutical composition with one or more compounds of this invention to a human or other mammal.

Another embodiment of this invention is a method for treating diseases in humans and/or other mammals which are characterized by abnormal angiogenesis or hyperpermiability processes, which are not raf-mediated, which comprises administering a compound of this invention or a pharmaceutical composition with one or more compounds of this invention to a human or other mammal.

Another embodiment of this invention is a method for treating diseases in humans and/or other mammals which are characterized by abnormal angiogenesis or hyperpermiability processes, which are not raf mediated or p38-mediated, which comprises administering a compound of this invention or a pharmaceutical composition with one or more compounds of this invention to a human or other mammal.

Another embodiment of this invention is a method for treating diseases in humans and/or other mammals which are characterized by abnormal angiogenesis or hyperpermiability processes, which are raf-mediated and/or p38 mediated, which comprises administering a compound of this invention or a pharmaceutical composition with one or more compounds of this invention to a human or other mammal.

Another embodiment of this invention is a method for treating one or more of the following conditions in humans and/or other mammals: tumor growth, retinopathy, including diabetic retinopathy, ischemic retinal-vein occlusion, retinopathy of prematurity and age related macular degeneration; rheumatoid arthritis, psoriasis, or a bullous disorder associated with subepidermal blister formation, including bullous pemphigoid, erythema multiforme, or dermatitis herpetiformis, which comprises administering a compound of this invention or a pharmaceutical composition with one or more compounds of this invention to a human or other mammal with one or more of these conditions.

A compound according to the invention can be administered simultaneously with another angiogenesis inhibiting agent to a patient with such a disorder, in the same formulation or, more typically in separate formulations and, often, using different administration routes. Administration can also be sequentially, in any order.

A compound according to the invention can be administered in tandem with another angiogenesis inhibiting agent, wherein a compound according to the invention can be administered to a patient once or more per day for up to 28 consecutive days with the concurrent or intermittent administration of another angiogenesis inhibiting agent over the same total time period.

A compound according to the invention can be administered to a patient at an oral, intravenous, intramuscular, subcutaneous, or parenteral dosage which can range from about 0.1 to about 200 mg/kg of total body weight and the additional angiogenesis inhibiting agent can be administered to a patient at an intravenous, intramuscular, subcutaneous, or parenteral dosage which can range from about 0.1 mg to 200 mg/kg of patient body weight.

This invention further relates to kits comprising separate doses of the two mentioned chemotherapeutic agents in separate containers. The combinations of angiogenesis inhibiting agents can also be formed in vivo, e.g., in a patient's body.

These additional angiogenesis inhibiting agents can be administered in the conventional formulations and regimens in which they are known for use alone.

Other conditions within a human or other mammal which can be treated by administering a compound of this invention include rheumatoid arthritis, osteoarthritis, septic arthritis, tumor metastasis, periodontal disease, cornal ulceration, proteinuria, coronary thrombosis from atherosclerotic plaque, aneurismal aortic, birth control, dystrophobic epidermolysis bullosa, degenerative cartilage loss following traumatic joint injury, osteopenias mediated by MMP activity, tempero mandibular joint disease or demyelating disease of the nervous system. Embodiments of this invention include the treatment of these conditions.

The compounds of this invention can also treat infectious diseases such as tuberculosis, Helicobacter pylori infection during peptic ulcer disease, Chaga's disease resulting from Trypanosoma cruzi infection, effects of Shiga-like toxin resulting from E. coli infection, effects of enterotoxin A resulting from Staphylococcus infection, meningococcal infection, and infections from Borrelia burgdorferi, Treponema pallidum, cytomegalovirus, influenza virus, Theiler's encephalomyelitis virus, and the human immunodeficiency virus (HIV) Embodiments of this invention include the treatment of these diseases.

The compounds of this invention can be made according to conventional chemical methods, and/or as disclosed below, from starting materials which are either commercially available or producible according to routine, conventional chemical methods. General methods for the preparation of the compounds are given below, and the preparation of a suitable compound is specifically illustrated in the Examples.

Ureas of formula (I) can be prepared by a variety of simple methods known in the art. General approaches for the formation of those compounds can be found in “Advanced Organic Chemistry”, by J. March, John Wiley and Sons, 1985 and in “Comprehensive Organic Transformations”, by R. C. Larock, VCH Publishers, 1989), which are hereby incorporated by reference. Nevertheless, the following general preparative methods are presented to aid one of skill in the art in synthesizing these compounds, with more detailed examples being presented in the experimental section describing the working examples.

General Preparative Methods

Heterocyclic amines may be synthesized utilizing known methodology (Katritzky, et al. Comprehensive Heterocyclic Chemistry; Permagon Press: Oxford, UK (1984). March. Advanced Organic Chemistry, 3^{rd} Ed.; John Wiley: New York (1985)). For example, as shown in Scheme I, 5-aminopyrazoles substituted at the N-1 position with either aryl or heteroaryl moieties may be synthesized by the reaction of an α-cyanoketone (2) with the appropriate aryl- or heteroaryl hydrazine (3, R²=aryl or heteroaryl). Cyanoketone 2, in turn, is available from the reaction of acetamidate ion with an appropriate acyl derivative, such as an ester, an acid halide, or an acid anhydride. In cases where the R² moiety offers suitable anion stabilization, 2-aryl and 2-heteroarylfurans may be synthesized from a Mitsunobu reaction of cyanoketone 2 with alcohol 5, followed by base catalyzed cyclization of enol ether 6 to give furylamine 7.

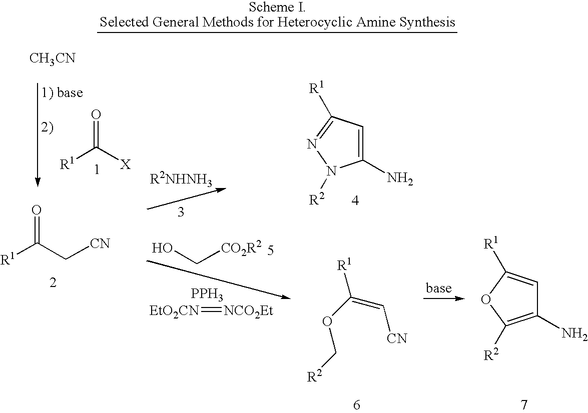

Substituted anilines may be generated using standard methods (March. Advanced Organic Chemistry, 3^{rd} Ed.; John Wiley: New York (1985). Larock. Comprehensive Organic Transformations; VCH Publishers: New York (1989)). As shown in Scheme II, aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as LiAlH₄ (Seyden-Penne. Reductions by the Alumino-and Borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)).

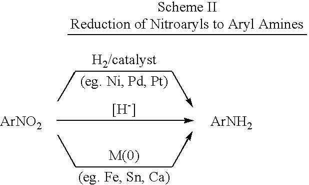

Nitroaryls are commonly formed by electrophilic aromatic nitration using HNO₃, or an alternative NO₂⁺ source. Nitro aryls may be further elaborated prior to reduction. Thus, nitroaryls substituted with

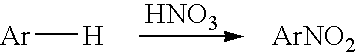

potential leaving groups (eg. F, Cl, Br, etc.) may undergo substitution reactions on treatment with nucleophiles, such as thiolate (exemplified in Scheme III) or phenoxide. Nitroaryls may also undergo Ullman-type coupling reactions (Scheme III).

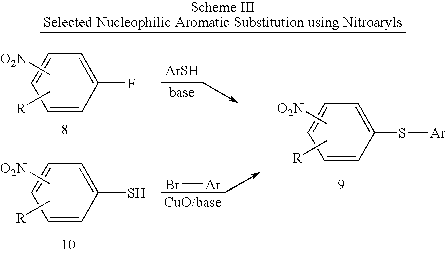

As shown in Scheme IV, urea formation may involve reaction of a heteroaryl isocyanate (12) with an aryl amine (11). The heteroaryl isocyanate may be synthesized from a heteroaryl amine by treatment with phosgene or a phosgene equivalent, such as trichloromethyl chloroformate (diphosgene), bis(trichloromethyl) carbonate (triphosgene), or N,N′-carbonyldiimidazole (CDI). The isocyanate may also be derived from a heterocyclic carboxylic acid derivative, such as an ester, an acid halide or an anhydride by a Curtius-type rearrangement. Thus, reaction of acid derivative 16 with an azide source, followed by rearrangement affords the isocyanate. The corresponding carboxylic acid (17) may also be subjected to Curtius-type rearrangements using diphenylphosphoryl azide (DPPA) or a similar reagent. A urea may also be generated from the reaction of an aryl isocyanate (15) with a heterocyclic amine.

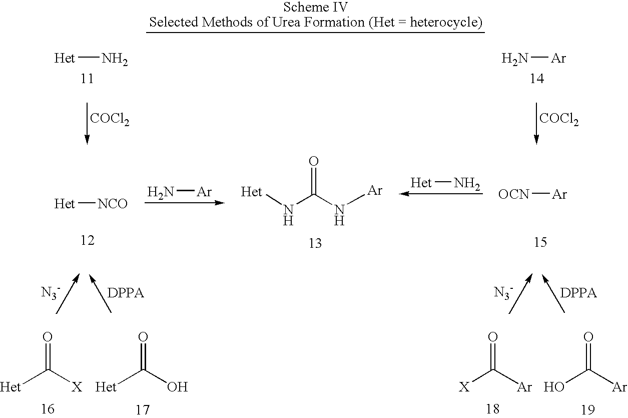

Finally, ureas may be further manipulated using methods familiar to those skilled in the art. For example, 2-aryl and 2-heteroarylthienyl ureas are available from the corresponding 2-halothienyl urea through transition metal mediated cross coupling reactions (exemplified with 2-bromothiophene 25, Scheme V). Thus, reaction of nitrile 20 with an α-thioacetate ester gives 5-substituted-3-amino-2-thiophenecarboxylate 21 (Ishizaki et al. JP 6025221). Decarboxylation of ester 21 may be achieved by protection of the amime, for example as the tert-butoxy (BOC) carbamate (22), followed by saponification and treatment with acid. When BOC protection is used, decarboxylation may be accompanied by deprotection giving the substituted 3-thiopheneammonium salt 23. Alternatively, ammonium salt 23 may be directly generated through saponification of ester 21 followed by treatment with acid. Following urea formation as described above, bromination affords penultimate halothiophene 25. Palladium mediated cross coupling of thiophene 25 with an appropriate tributyl- or trimethyltin (R²=aryl or heteroaryl) then affords the desired 2-aryl- or 2-heteroarylthienyl urea.

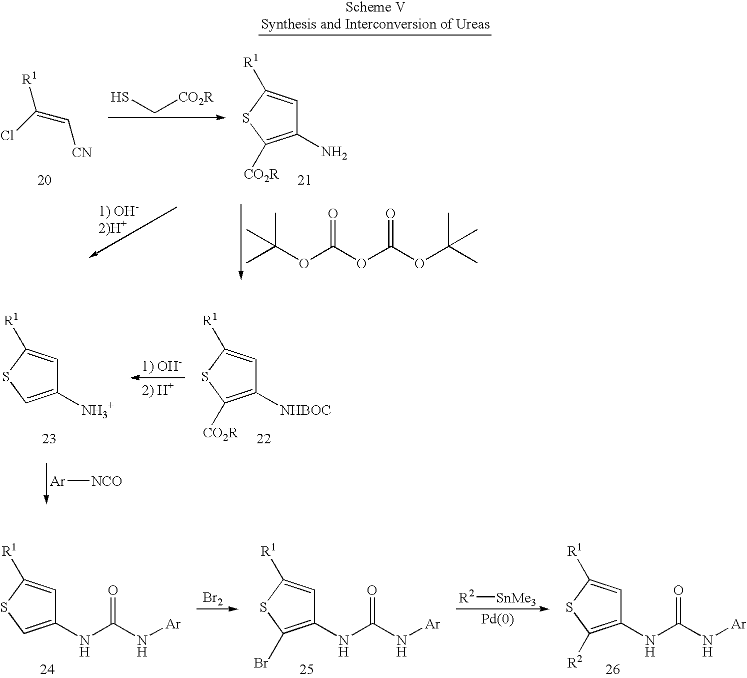

Finally, ureas may be further manipulated using methods familiar to those skilled in the art.

The compounds may be administered orally, topically, parenterally, by inhalation or spray or vaginally, sublingually, or rectally in dosage unit formulations. The term ‘administration by injection’ includes intravenous, intramuscular, subcutaneous and parenteral injections, as well as use of infusion techniques. Dermal administration may include topical application or transdermal administration. One or more compounds may be present in association with one or more non-toxic pharmaceutically acceptable carriers and if desired other active ingredients.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in to order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, microcrystalline cellulose, carboxymethyl cellulose, hydroxypropylmethylcellulose or alginic acid; and binding agents, for example magnesium stearate, stearic acid or talc and lubricants/surfactants such as sodium lauryl sulfate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally occurring phosphatide, for example, lecithin, or condensation products or an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example sum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal or vaginal temperature and will therefore melt in the rectum or vagina to release the drug. Such materials include cocoa butter and polyethylene glycols.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a compound of Formula I in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound, of Formula I may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl isobutyl tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene coploymers, and natural and synthetic rubbers. Cellulose ethers, derivatized polyethylenes, and silicates may also be used as matrix components. Additional additives, such as viscous resins or oils may be added to increase the viscosity of the matrix.

For all regimens of use disclosed herein for compounds of Formula I, the daily oral dosage regimen will preferably be from 0.01 to 200 mg/Kg of total body weight. The daily dosage for administration by injection, including intravenous, intramuscular, subcutaneous and parenteral injections, and use of infusion techniques will preferably be from 0.01 to 200 mg/Kg of total body weight. The daily vaginal dosage regimen will preferably be from 0.01 to 200 mg/Kg of total body weight. The daily rectal dosage regime will preferably be from 0.01 to 200 mg/Kg of total body weight. The daily topical dosage regime will preferably be from 0.1 to 200 mg administered between one to four times daily. The transdermal concentration will preferably be that required to maintain a daily dose of from 0.01 to 200 mg/Kg. The daily inhalation dosage regime will preferably be from 0.01 to 10 mg/Kg of total body weight. These dosages regimes can be achieved with multiple dosages within a single day or extended dosages, such as those given on a weekly or monthly basis.

It will be appreciated by those skilled in the art that the particular method of administration will depend on a variety of factors, all of which are considered routinely when administering therapeutics. It will also be appreciated by one skilled in the art that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy.

It will be further appreciated by one skilled in the art that the optimal course of treatment, i.e., the mode of treatment and the daily number of doses of a compound of this invention given for a defined number of days, can be ascertained by those skilled in the art using conventional treatment tests.

It will be understood, however, that the specific dose level for any particular patient will depend upon a variety of factors, including the activity of the specific compound employed, the age, body weight, general health, sex, diet, time of administration, route of administration, and rate of excretion, drug combination and the severity of the condition undergoing therapy.

Specific preparations of the compounds of this invention are already described in the patent literature, and can be adapted to the compounds of the present invention such as, for example, WO 99/23091, WO 00/43384, WO 00/55139, WO 00/55152 and WO 01/36403, which are incorporated herein by reference.

The entire disclosure of all applications, patents and publications cited above and below are hereby incorporated by reference.

The compounds of this invention are producible from known compounds (or from starting materials which, in turn, are producible from known compounds), e.g., through the general preparative methods shown below. The activity of a given compound to inhibit angiogenesis activity can be routinely assayed, e.g., according to procedures disclosed below.

Without further elaboration, it is believed that one skilled in the art can, using the preceding description, utilize the present invention to its fullest extent. The following examples are, therefore, to be construed as merely illustrative and not limitative of the remainder of the disclosure in any way whatsoever. The following examples are for illustrative purposes only and are not intended, nor should they be construed to limit the invention in any way.

### EXAMPLES

All reactions were performed in flame-dried or oven-dried glassware under a positive pressure of dry argon or dry nitrogen, and were stirred magnetically unless otherwise indicated. Sensitive liquids and solutions were transferred via syringe or cannula, and introduced into reaction vessels through rubber septa. Unless otherwise stated, the term ‘concentration under reduced pressure’ refers to use of a Buchi rotary evaporator at approximately 15 mmHg.

All temperatures are reported uncorrected in degrees Celsius (° C.). Unless otherwise indicated, all parts and percentages are by weight.

Commercial grade reagents and solvents were used without further purification.

Thin-layer chromatography (TLC) was performed on Whatman® pre-coated glass-backed silica gel 60A F-254 250 μm plates. Visualization of plates was effected by one or more of the following techniques: (a) ultraviolet illumination, (b) exposure to iodine vapor, (c) immersion of the plate in a 10% solution of phosphomolybdic acid in ethanol followed by heating, (d) immersion of the plate in a cerium sulfate solution followed by heating, and/or (e) immersion of the plate in an acidic ethanol solution of 2,4-dinitrophenylhydrazine followed by heating. Column chromatography (flash chromatography) was performed using 230-400 mesh EM Science® silica gel.

Melting points (mp) were determined using a Thomas-Hoover melting point apparatus or a Mettler FP66 automated melting point apparatus and are uncorrected. Proton (¹H) nuclear magnetic resonance (NMR) spectra were measured with a General Electric GN-Omega 300 (300 MHz) spectrometer with either Me₄Si (δ 0.00) or residual protonated solvent (CHCl₃ δ 7.26; MeOH δ 3.30; DMSO δ 2.49) as standard. Carbon (¹³C) NMR spectra were measured with a General Electric GN-Omega 300 (75 MHz) spectrometer with solvent (CDCl₃δ 77.0; MeOD-d₃; δ 49.0; DMSO-d₆δ 39.5) as standard. Low resolution mass spectra (MS) and high resolution mass spectra (HRMS) were either obtained as electron impact (EI) mass spectra or as fast atom bombardment (FAB) mass spectra. Electron impact mass spectra (EI-MS) were obtained with a Hewlett Packard 5989A mass spectrometer equipped with a Vacumetrics Desorption Chemical Ionization Probe for sample introduction. The ion source is maintained at 250° C. Electron impact ionization was performed with electron energy of 70 eV and a trap current of 300 μA. Liquid-cesium secondary ion mass spectra (FAB-MS), an updated version of fast atom bombardment, were obtained using a Kratos Concept 1-H spectrometer.

Chemical ionization mass spectra (CI-MS) were obtained using a Hewlett Packard MS-Engine (5989A) with methane as the reagent gas (1×10⁻⁴ torr to 2.5×10⁻⁴ torr). The direct insertion desorption chemical ionization (DCI) probe (Vaccumetrics, Inc.) was ramped from 0-1.5 amps in 10 sec and held at 10 amps until all traces of the sample disappeared (˜1-2 min). Spectra were scanned from 50-800 amu at 2 sec per scan. HPLC—electrospray mass spectra (HPLC ES-MS) were obtained using a Hewlett-Packard 1100 HPLC equipped with a quaternary pump, a variable wavelength detector, a C-18 column, and a Finnigan LCQ ion trap mass spectrometer with electrospray ionization. Spectra were scanned from 120-800 amu using a variable ion time according to the number of ions in the source.

Gas chromatography—ion selective mass spectra (GC-MS) were obtained with a Hewlett Packard 5890 gas chromatograph equipped with an HP-1 methyl silicone column (0.33 mM coating; 25 m×0.2 mm) and a Hewlett Packard 5971 Mass Selective Detector (ionization energy 70 eV).

Elemental analyses were conducted by Robertson Microlit Labs, Madison N.J. All compounds displayed NMR spectra, LRMS and either elemental analysis or HRMS consistent with assigned structures.

List of Abbreviations and Acronyms:

| | |
|---|---|
| AcOH | acetic acid |
| anh | anhydrous |
| BOC | tert-butoxycarbonyl |
| conc | concentrated |
| dec | decomposition |
| DMPU | 1,3-dimethyl-3,4,5,6-tetrahydro-2(1H)-pyrimidinone |
| DMF | N,N-dimethylformamide |
| DMSO | dimethylsulfoxide |
| DPPA | diphenylphosphoryl azide |
| EtOAc | ethyl acetate |
| EtOH | ethanol (100%) |
| Et₂O | diethyl ether |
| Et₃N | triethylamine |
| m-CPBA | 3-chloroperoxybenzoic acid |
| MeOH | methanol |
| pet. ether | petroleum ether (boiling range 30-60° C.) |
| THF | tetrahydrofuran |
| TFA | trifluoroacetic acid |
| Tf | trifluoromethanesulfonyl |

### Experimental

Preparation of 3-tert-butyl-1-(4-methylphenyl)-1H-pyrazol-5-yl amine

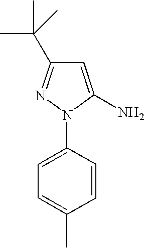

To a solution of 4,4-dimethyl-3-oxopentanenitrile (4.4 g, 34.8 mmol) and p-tolylhydrazine hydrochloride (5.0 g, 31.5 mmol) in abs EtOH (75 mL) was added conc. HCl (3.5 mL). The reaction was heated at the reflux temperature overnight. The resulting mixture was concentrated under reduced pressure and the solid residue obtained was washed with Et₂O. The solid was then suspended in EtOAc and treated with a saturated NaHCO₃ solution. The organic layer was washed with a saturated NaCl solution, and dried (MgSO₄). The solid residue obtained was washed with hexanes to afford 3-tert-butyl-1-(4-methylphenyl)-1H-pyrazol-5-ylamine (4.68 g, 59%): TLC (20% EtOAc/hexane) R_{f} 0.16; ¹H NMR (DMSO-d₆) δ 1.20 (s, 9H), 2.32 (s, 3H), 5.10 (br s, 2H), 5.35 (s, 1H), 7.23 (d, J=8.3 Hz, 2H), 7.42 (d, J=8.7 Hz, 2H).

Preparation of 4-(4-pyridinylmethoxy)-1-naphthalenamine

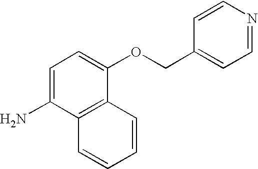

Step 1. Preparation of tert-butyl 4-hydroxy-1-naphthylcarbamate

A mixture of 4-amino-1-naphthol hydrochloride (6 g, 27.6 mmol) in dry THF (30 mL) was cooled to −78° C. under argon. Butyllithium (1.6 M in hexanes; 17 mL, 27.2 mmol) was added dropwise via syringe. After the addition was complete, the reaction was allowed to warm up to room temperature and was stirred for 30 minutes. The mixture was then cooled again to −78° C. and treated with a solution of di-tert-butyl dicarbonate (6.7 g, 30.4 mmol) in THF (7 mL). The reaction mixture was allowed to warm slowly to room temperature and was stirred at that temperature for 3 h. The reaction mixture was then diluted with EtOAc, sequentially washed with H₂O and a saturated NaCl solution, and dried (MgSO₄). The mixture was concentrated under reduced pressure and the residue obtained was treated with hexanes and sonicated. The solid that formed was removed and dried to afford tert-butyl 4-hydroxy-1-naphthylcarbamate (6 g, 85%) TLC (30% EtOAc/hexane) R_{f} 0.44; ¹H NMR (DMSO-d₆) δ 1.45 (s, 9H), 6.79 (d, J=8.4 Hz, 1H), 7.20 (d, J=7.9 Hz, 1H), 7.40-7.51 (m, 2H), 7.82-7.85 (m, 1H), 8.09-8.13 (m, 1H), 8.79 (br s, 1H), 10.04 (s, 1H).

Step 2. Preparation of tert-butyl 4-(4-pyridinylmethoxy)-1-naphthylcarbamate

A solution of tert-butyl 4-hydroxy-1-naphthylcarbamate (1.1 g, 4.24 mmol) in dry CH₃CN (20 mL) was treated with powdered K₂CO₃ (2.7 g, 19.5 mmol) and 4-picolyl chloride hydrochloride (0.67 g, 3.96 mmol). The reaction mixture was heated at 80° C. under argon for 2.5 hr. TLC analysis showed almost complete conversion to product. The reaction mixture was then cooled to room temperature and quenched with EtOAc and H₂O. The organic layer was washed with a saturated NaCl solution and dried (MgSO₄). The residue obtained was purified by MPLC (Biotage® gradient from 40% EtOAc/hexane to 60% EtOAc/hexane) to afford tert-butyl 4-(4-pyridinylmethoxy)-1-naphthylcarbamate as an off-white solid (1.22 g, 88%): TLC (50% EtOAc/hexane) R_{f} 0.21; ¹H NMR (DMSO-d₆) δ 1.46 (s, 9H), 5.38 (s, 2H), 6.98 (d, J=7.9 Hz, 1H), 7.35 (d, J=8.5 Hz, 1H), 7.51-7.60 (m, 4H), 7.91-7.95 (m, 1H), 8.27-8.31 (m, 1H) 8.60 (d, J=5.8 Hz, 2H), 8.97 (br s, 1H).

Step 3. Preparation of 4-(4-pyridinylmethoxy)-1-naphthalenamine

A solution of 4 N HCl in 1,4-dioxane (10 mL) was added to tert-butyl 4-(4-pyridinylmethoxy)-1-naphthylcarbamate (0.96 g, 2.74 mmol). The reaction mixture was stirred at room temperature for 2 h at which time TLC analysis showed complete reaction. The mixture was suspended in EtOAc and treated with a 1 N NaOH solution. The organic layer was washed with a saturated NaCl solution and dried (MgSO₄) to afford 4-(4-pyridinylmethoxy)-1-naphthalenamine as a brown solid (0.58 g, 85%): TLC (60% EtOAc/hexane) R_{f} 0.19; ¹H NMR (DMSO-d₆) δ 5.22 (s, 2H), 5.25 (br s, 2H), 6.57 (d, J=8.3 Hz, 1H), 6.81 (d, J=8.2 Hz, 1H), 7.40-7.52 (m, 4H), 8.01-8.04 (m, 1H), 8.14-8.18 (m, 1H), 8.58 (d, J=5.7 Hz, 2H).

### Example 1

Preparation of 1-[5-tert-butyl-2-p-tolyl-2H-pyrazol-3-yl]-3-[4-(pyridin-4-yl-methoxy)naphthalen-1-yl]-urea

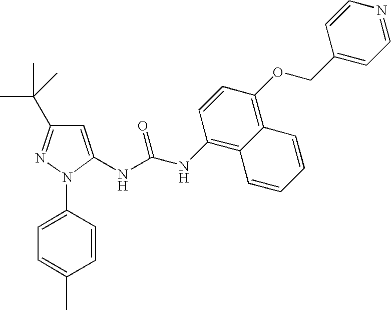

A mixture of 3-tert-butyl-1-(4-methylphenyl)-1H-pyrazol-5-ylamine (79 mg, 0.344 mmol) in CH₂Cl₂ (10 mL), and a saturated NaHCO₃ solution (10 mL) was cooled to 0° C. and treated with phosgene (1.93 M in toluene; 0.7 mL, 1.35 mmol). The resulting mixture was stirred at 0° C. for 15 min. The organic layer was separated and dried (MgSO₄). The residue was then dissolved in THF (2 mL) and added to a solution of 4-(4-pyridinylmethoxy)-1-naphthalenamine (80 mg, 0.32 mmol) in THF (5 mL). The reaction mixture was stirred at room temperature for 3 h. TLC analysis showed complete conversion. The reaction mixture was diluted with EtOAc, sequentially washed with H₂O and a saturated NaCl solution, and dried (MgSO₄). The residue was then absorbed onto silica then purified by MPLC (Biotage®; gradient from 70% EtOAc/hexane to 100% EtOAc) to afford 1-[5-tert-butyl-2-p-tolyl-2H-pyrazol-3-yl]-3-[4-(pyridin-4-yl-methoxy)naphthalen-1-yl]-urea as a solid (118 mg, 68%): TLC (60% EtOAc/hexane) R_{f} 0.16; ¹H NMR (DMSO-d₆) δ 1.26 (s, 9H), 2.38 (s, 3H), 5.37 (s, 2H), 6.35 (s, 1H), 7.00 (d, J=8.5 Hz, 1H), 7.35 (d, J=8.3 Hz, 2H), 7.43 (d, J=8.2 Hz, 2H), 7.53-7.64 (m, 5H), 7.89-7.93 (m, 1H), 8.29-8.32 (m, 1H), 8.58 (s, 1H), 8.60-8.62 (m, 2H), 8.78 (s, 1H); HPLC EI-MS m/z 506 ((M+H)⁺).

### Example 2

Preparation of 1-[5-tert-butyl-2-(4-isopropylphenyl)-2H-pyrazol-3-yl]-3-[4-(pyridin-4-yl-methoxy)naphthalen-1-yl]-urea

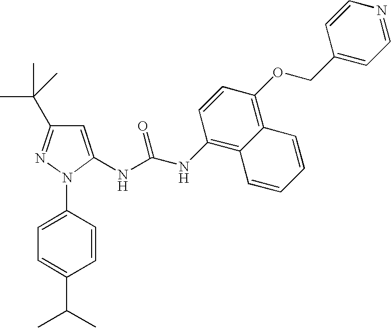

1-[5-tert-Butyl-2-(4-isopropylphenyl)-2H-pyrazol-3-yl]-3-[4-(pyridin-4-yl-methoxy)naphthalen-1-yl]-urea was prepared in a manner analogous to that for the synthesis of 1-[5-tert-butyl-2-p-tolyl-2H-pyrazol-3-yl]-3-[4-(pyridin-4-yl-methoxy)naphthalen-1-yl]-urea except 4-isopropylphenylhydrazine was used in place of p-tolylhydrazine: TLC (60% EtOAc/hexane) R_{f} 0.21: HPLC EI-MS m/z ((M+H)⁺).

### Example 3

Preparation of 1-[5-tert-butyl-2-cyclohexyl-2H-pyrazol-3-yl]-3-[4-(pyridin-4-yl-methoxy)naphthalen-1-yl]-urea

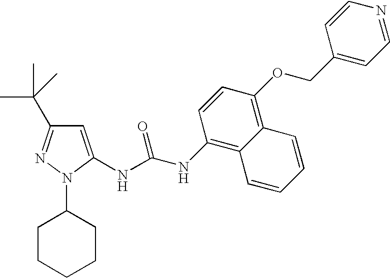

1-[5-tert-butyl-2-cyclohexyl-2H-pyrazol-3-yl]-3-[4-(pyridin-4-yl-methoxy)naphthalen-1-yl]-urea was prepared in a manner analogous to that for the synthesis of 1-[5-tert-butyl-2-p-tolyl-2H-pyrazol-3-yl]-3-[4-(pyridin-4-yl-methoxy)naphthalen-1-yl]-urea except cyclohexylhydrazine was used in place of p-tolylhydrazine: TLC (60% EtOAc/hexane) R_{f} 0.09; HPLC EI-MS m/z 498 ((M+H)⁺).

### Example 4

Preparation of 1-[5-tert-butyl-2-(2-methylphenyl)-2H-pyrazol-3-yl]-3-[4-(pyridin-4-yl-methoxy)naphthalen-1-yl]-urea

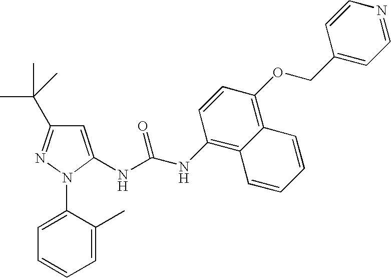

1-[5-tert-butyl-2-(2-methylphenyl)-2H-pyrazol-3-yl]-3-[4-(pyridin-4-yl-methoxy)naphthalen-1-yl]-urea was prepared in a manner analogous to that for the synthesis of 1-[5-tert-butyl-2-p-tolyl-2H-pyrazol-3-yl]-3-[4-(pyridin-4-yl-methoxy)naphthalen-1-yl]-urea except 2-methylphenylhydrazine was used in place of p-tolylhydrazine: TLC (60% EtOAc/hexane) R_{f} 0.09; HPLC EI-MS m/z 506 ((M+H)⁺).

### Example 5

Preparation of 1-[5-tert-butyl-2-(3-trifluoromethylphenyl)-2H-pyrazol-3-yl]-3-[4-(pyridin-4-yl-methoxy)naphthalen-1-yl]-urea

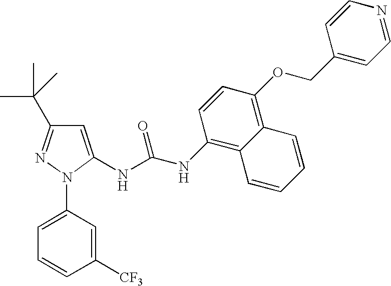

1-[5-tert-butyl-2-(3-trifluoromethylphenyl)-2H-pyrazol-3-yl]-3-[4-(pyridin-4-yl-methoxy)naphthalen-1-yl]-urea was prepared in a manner analogous to that for the synthesis of 1-[5-tert-butyl-2-p-tolyl-2H-pyrazol-3-yl]-3-[4-(pyridin-4-yl-methoxy)naphthalen-1-yl]-urea except 3-trifluoromethylphenylhydrazine was used in place of p-tolylhydrazine: TLC (60% EtOAc/hexane) R_{f} 0.30; HPLC EI-MS m/z 560 ((M+H)⁺).

### Example 6

Preparation of {[3-(tert-butyl)-1-(4-methylphenyl)pyrazol-5-yl]amino}-N-[4-(2-morpholin-4-ylethoxy)naphthyl]carboxamide-[5-tert-butyl-2-(3-trifluoromethylphenyl)-2H-pyrazol-3-yl]-3-[4-(pyridin-4-yl-methoxy)naphthalen-1-yl]-urea

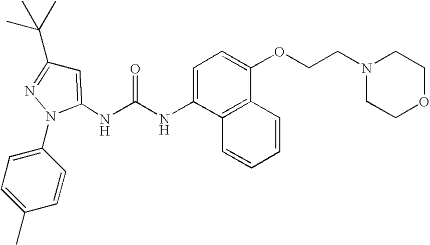

Step 1 Synthesis of 4-nitronaphthol

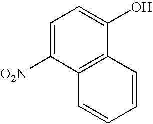

A solution of 4-methoxy-1-nitronaphthalene (50.0 g, 0.25 mol) and NaI (184 g, 1.23 mol) in DMF (1.5 L) was heated at 120° C. overnight while bubbling Ar through reaction mixture and into a trap to collect MeI. The resulting reaction was divided into two equal volume batches. The first batch was diluted with H₂O (500 mL), a 1N NaOH solution (500 mL) and washed with EtOAc (2×500 mL). The aqueous layer was acidified to pH 2 using a con. HCl solution and extracted with EtOAc (2×500 mL). The second batch was handled the same way. The final organic layers were combined and concentrated under reduced pressure. The residue was dissolved in CH₂Cl₂, and filtered through a pad of silica (gradient from hexanes to 15% EtOAc/hex) to obtain 4-nitronaphthol (21.5 g, 46%) an orange-brown solid: TLC (30% EtOAc/hex) R_{f} 0.25.

Step 2 Synthesis of 4-(2-morpholin-4-ylethoxy)-1-nitronaphthalene

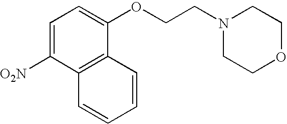

A mixture of 4-nitronaphthol (10.0 g, 52.9 mmol), 4-(2-chloroethyl)morpholine hydrochloride (13.8 g, 74.0 mmol), NaOH (3.0 g, 74.0 mmol) and K₂CO₃ (17.5 g, 126.9 mmol) in NMP (50 mL) was heated at 100° C. for 3 h, then was stirred at room temperature overnight. The resulting mixture was diluted with H₂O (150 mL) and stirred for 2 h. More H₂O (200 mL) was added. The resulting precipitate was filtered, washed with H₂O and dried under reduced pressure to obtain 4-(2-morpholin-4-ylethoxy)-1-nitronaphthalene (11.5 g, 72%) as a brown solid: TLC (EtOAc) R_{f}0.20; ES-LCMS (rel abundance) m/z 303 ((M+1)⁺, 100%).

Step 3 Synthesis of 4-(2-morpholin-4-ylethoxy)naphthylamine

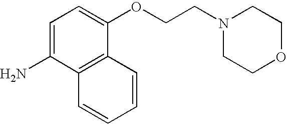

Pd/C was placed under Ar atmosphere. MeOH (50 mL) was added, followed by 4-(2-morpholin-4-ylethoxy)-1-nitronaphthalene (10.4 g, 34.4 mmol). The mixture was placed under H₂ atmosphere (1 atm) for 16 h. The resulting mixture was filtered through a pad of Celite and washed with MeOH. The filtrate was concentrated under reduced pressure. The residue was dissolved in CH₂Cl₂ and purified by MPLC (Flash 40S; EtOAc) to give 4-(2-morpholin-4-ylethoxy)naphthylamine (8.2 g, 87%) as a brown oil: TLC (EtOAc) R_{f}0.18; ES-LCMS (rel abundance) m/z 273 ((M+1)⁺,100%).

Step 4 Synthesis of {[3-(tert-butyl)-1-(4-methylphenyl)pyrazol-5-yl]amino}-N-[4-(2-morpholin-4-ylethoxy)naphthyl]carboxamide

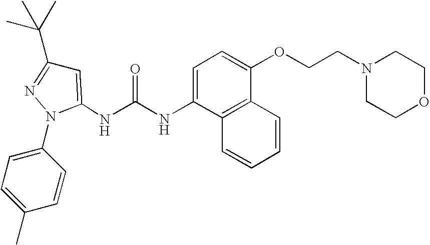

To a solution of CDI (3.53 g, 21.8 mmol) in CH₂Cl₂ (4 mL) was added dropwise a solution of 3-(tert-butyl)-1-(4-methylphenyl)pyrazole-5-ylamine (5.00 g, 21.8 mmol) in CH₂Cl₂ (10 mL) over 1 h. The resulting slurry was stirred for 2 h, then treated with the aniline (2.97 g, 10.9 mmol) in CH₂Cl₂ (4 mL). The deep brown mixture was stirred overnight. The resulting mixture was concentrated under reduced pressure. The residue was diluted with CH₂Cl₂ and purified by MPLC (Flash 75M; EtOAc). The product was further purified by HPLC (C18, iPrOH/hex) to give {[3-(tert-butyl)-1-(4-methylphenyl)pyrazol-5-yl]amino}-N-[4-(2-morpholin-4-ylethoxy)naphthyl]carboxamide (4.6 g, 80%) as a light brown solid: TLC (EtOAc) R_{f} 0.18; ES-LCMS (rel abundance) m/z 528 ((M+1)⁺,100%).

### Biological Examples

In Vitro Raf Kinase Assay

In an in vitro kinase assay, raf was incubated with MEK in 20 mM Tris-HCl, pH 8.2 containing 2 mM, 2-mercaptoethanol and 100 mM NaCl. This protein solution (20 μL) was mixed with water (5 μL) or with compounds diluted with distilled water from 10 mM stock solutions of compounds dissolved in DMSO. The kinase reaction was initiated by adding 25 μL [γ-³³P]ATP (1000-3000 dpm/pmol) in 80 mM Tris-HCl, pH 7.5, 120 mM NaCl, 1.6 mM DTT, 16 mM MgCl₂. The reaction mixtures were incubated at 32° C., usually for 22 min. Incorporation of ³³P into protein was assayed by harvesting the reaction onto phosphocellulose mats, washing away free counts with a 1% phosphoric acid solution and quantitating phosphorylation by liquid scintillation counting. For high throughput screening, 10 μM ATP and 0.4 μM MEK was used. In some experiments, the kinase reaction was stopped by adding an equal amount of Laemmli sample buffer. Samples were boiled 3 min and the proteins resolved by electrophoresis on 7.5% Laemmli gels. Gels were fixed, dried and exposed to an imaging plate (Fuji). Phosphorylation was analyzed using a Fujix Bio-Imaging Analyzer System.

{[3-(tert-Butyl)-1-(4-methylphenyl)pyrazol-5-yl]amino}-N-[4-(2-morpholin-4-ylethoxy)naphthyl]carboxamide displayed an IC₅₀ of less than 1 micromolar in this assay.

In Vitro Flk-1 ELISA Assay

The Flk-1 ELISA assay was performed on 96-well Costar 9018 high-binding plates coated overnight with poly-Glu-Tyr (Sigma #P 0275). After coating, plates were washed with 20 mM HEPES pH=7.5, 150 mM NaCl, 0.1% Tween-20. These plates were then blocked with Blocking Buffer (20 mM HEPES pH=7.5, 150 mM NaCl, 1% BSA, 0.1% Tween-20) for 1 hr at room temperature on a plate shaker. After washing, the reaction was prepared using 20 mM HEPES pH=7.5, 100 mM KCl, 10 mM MgCl₂, 3 mM MnCl₂ 0.05% glycerol, 0.005% Triton X-100, 1 mM BME and 3.3 μM ATP. The assay was initiated with Flk-1 giving a final enzyme concentration of 50 ng/well.

The reaction was incubated at room temperature for 1 hr on a plate shaker and was stopped by washing the plate. The primary antibody incubation step was for 1 hour at room temperature using PT66 phosphotyrosine antibody (Sigma 1:5000 dilution). After washing, the plate was then incubated (1 hr room temp) with the secondary antibody solution of anti-mouse HRP-conjugated antibody (Amersham 1:5000 dilution). After another wash step, plates were developed using 100 μL TMB substrate solution (KPL) and then quenched with KPL stop solution. These quenched plates were read at 450 nM on a Molecular Devices spectrophotomer plate reader. For IC₅₀ generation, compounds were added prior to the enzyme initiation. A 50-fold stock plate was made with compounds serially diluted 1:3 in a 50% DMSO/50% dH₂O solution. A 2 μL addition of the stock to the assay gave final compound concentrations ranging from 1 μM-0.456 nM in 1% DMSO. The data were expressed as percent inhibition: % inhibition=100-((O.D. with inhibitor-background)/(O.D.without inhibitor-background)) * 100.

{[3-(tert-Butyl)-1-(4-methylphenyl)pyrazol-5-yl]amino }-N- [4-(2-morpholin-4-ylethoxy)naphthyl]carboxamide displayed an IC₅₀ of less than 1 micromolar in this assay.

Cell Mechanistic Assay-Inhibition of 3T3 KDR Phosphorylation:

NIH3T3 cells expressing the full length KDR receptor are grown in DMEM (Life Technologies, Inc., Grand Island, N.Y.) supplemented with 10% newborn calf serum, low glucose, 25 mM/L sodium pyruvate, pyridoxine hydrochloride and 0.2 mg/ml of G418 (Life Technologies Inc., Grand Island, N.Y.). The cells are maintained in collagen I-coated T75 flasks (Becton Dickinson Labware, Bedford, Mass.) in a humidified 5% CO2 atmosphere at 37° C.

Fifteen thousand cells are plated into each well of a collagen I-coated 96-well plate in the DMEM growth medium. Six hours later, the cells are washed and the medium is replaced with DMEM without serum. After overnight culture to quiesce the cells,t he medium is replaced by Dulbecco's phosphate-buffered saline (Life Technologies Inc., Grand Island, N.Y.) with 0.1% bovine albumin (Sigma Chemical Co., St. Louis, Mo.). After adding various concentrations (0-300 nM) of test compounds to the cells in 1% final concentration of DMSO, the cells are incubated at room temperature for 30 minutes. Following VEGF stimulation, the buffer is removed and the cells are lysed by addition of 150 μl of extraction buffer (50 mM Tris, pH 7.8, supplemented with 10% glycerol, 50 mM BGP, 2 mM EDTA, 10 mM NaF, 0.5 mM NaVO4, and 0.3% TX-100) at 4° C. for 30 minutes.

To assess receptor phosphorylation, 100 microliters of each cell lysate are added to the wells of an ELISA plate precoated with 300 ng of antibody C20 (Santa Cruz Biotechnology, Inc., Santa Cruz, Calif.). Following a 60-minute incubation, the plate is washed and bound KDR is probed for phosphotyrosine using an anti-phosphotyrosine mAb clone 4G10 (Upstate Biotechnology, Lake Placid, N.Y.). The plate is washed and wells are incubated with anti-mouse IgG/HRP conjugate (Amersham International plc, Buckinghamshire, and England) for 60 minutes. Wells are washed and phosphotyrosine is quantitated by addition of 100 μl per well of 3,3′,5,5′tetramethylbenzidine (Kirkegaard and Perry, TMB 1 Component Stop Solution).

Optical densities (OD) are determined spectrophotometrically at 450 mm in a 96-well plate reader (SpectraMax 250, Molecular Devices). Background (no VEGF added) OD values are subtracted from all Ods and percent inhibition is calculated according to the equation:

$$\% Inhibition = \frac{( OD ( VEGF control ) - OD ( with test compound ) \times 100}{OD ( VEGF control ) - OD ( no VEGF added )}$$

IC_{50S} are determined on some of the exemplary materials with at least squares analysis program using compound concentration versus percent inhibition.

Matrigel® Angiogenesis Model:

Preparation of Martigel Plugs and in vivo Phase: Matrigel® (Collaborative Biomedical Products, Bedford, Mass.) is a basement membrane extract from a murine tumor composed primarily of laminin, collagen IV and heparan sulfate proteoglycan. It is provided as a sterile liquid at 4° C., but rapidly from a solid gel at 37° C.

Liquid Matrigel at 4° C. is mixed with SK-MEL2 human tumor cells that are transfected with a plasmid containing the murine VEGF gene with a selectable marker. Tumor cells are grown in vitro under selection and cells are mixed with cold liquid Matrigel at a ratio of 2×10⁶ per 0.5 ml. One half milliliter is implanted subcutaneously near the abdominal midline using a 25 gauge needle. Test compounds are dosed as solutions in Ethanol/Ceremaphor EL/saline (12.5%:12.5%:75%) at 30, 100, and 300 mg/kg po once daily starting on the day of implantation. Mice are euthanized 12 days post-implantation and the Matrigel pellets are harvested for analysis of hemoglobin content.

Hemoglobin Assay: The Matrigel pellets are placed in 4 volumes (w/v) of 4° C. Lysis Buffer (20 mM Tris pH 7.5, 1 mM EGTA, 1 mM EDTA, 1% Triton X-100 [EM Science, Gibbstown, N.J.], and complete EDTA-free protease inhibitor cocktail [Mannheim, Germany]), and homogenized at 4° C. homogenates are incubated on ice for 30 minutes with shaking and centrifuged at 14K×g for 30 minutes at 4° C. Supernatants are transferred to chilled microfuge tubes and stored at 4° C. for hemoglobin assay.

Mouse hemoglobin (Sigma Chemical Co., St. Louis, Mo.) is suspended in autoclaved water (BioWhittaker, Inc, Walkersville, Md.) at 5 mg/ml. A standard curve is generated from 500 micrograms/ml to 30 micrograms/ml in Lysis Buffer (see above). Standard curve and lysate samples are added at 5 microliters/well in duplicate to a polystyrene 96-well plate. Using the Sigma Plasma Hemoglobin Kit (Sigma Chemical Co., St. Louis, Mo.), TMB substrate is reconstituted in 50 mls room temperature acetic acid solution. One hundred microliters of substrate is added to each well, followed by 100 microliters/well of Hydrogen Peroxide Solution at room temperature. The plate is incubated at room temperature for 10 minutes.

Optical densities are determined spectrophotometrically at 600 nm in a 96-well plate reader, SpectraMax 250 Microplate Spectrophotometer System (Molecular Devices, Sunnyvale, Calif.). Background Lysis Buffer readings are subtracted from all wells.

Total sample hemoglobin content is calculated according to the following equation:

Total Hemoglobin=(Sample Lysate Volume)×(Hemoglobin Concentration)

The average Total Hemoglobin of Matrigel samples without cells are subtracted from each Total Hemoglobin Matrigel sample with cells. Percent inhibition is calculated according to the following equation:

$$\% Inhibition = \frac{( Average Total Hemoglobin Drug \text{-} Treated Tumor Lysates ) \times 100}{( Average Total Hemoglobin Non \text{-} Treated Tumore Lysates )}$$

In Vivo Assay of Antitumor Activity:

An in vivo assay of the inhibitory effect of the compounds on tumors (e.g., solid cancers) mediated by raf kinase can be performed as follows: CDI nu/nu mice (6-8 weeks old) are injected subcutaneously into the flank at 1×10⁶ cells with human colon adenocarcinoma cell line. The mice are dosed i.p., i.v. or p.o. at 10, 30, 100, or 300 mg/Kg beginning on approximately day 10, when tumor size is between 50-100 mg. Animals are dosed for 14 consecutive days; tumor size is monitored with calipers twice a week. The inhibitory effect of the compounds on raf kinase and therefore on tumors (e.g., solid cancers) mediated by raf kinase can further be demonstrated in vivo according to the technique of Monia et al. (Nat. Med. 1996, 2, 668-75).

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various conditions and usages.

### Examples 1-283

The compounds listed in the tables below are described in the PCT applications identified in the heading for each table. These compounds can be prepared by the methods, described in these PCT applications, by an analogous procedure or other procedures well known to those skilled in the art. The PCT applications identified in the tables are incorporated herein by reference.

TABLE 1
WO 99/23091

| Example | |
|---|---|
| Number | Compound Name |
| 1 | 1-[5-tert-butyl-2-(2-methylpyridin-5-yl)-2H-pyrazol-3- |
| | yl]-3-(4-chlorophenyl)urea |
| 2 | 1-(5-tert-butyl-2-p-tolyl-2H-pyrazol-3-yl)-3-(4- |
| | methoxynaphthalen-1-yl)urea |
| 3 | 1-[5-tert-butyl-2-(3,4-dimethylphenyl)-2H-pyrazol-3-yl]-3-(4- |
| | fluorophenyl)urea |
| 4 | 1-[5-tert-butyl-2-(pyridin-3-yl)-2H-pyrazol-3-yl]-3-(4- |
| | cyanonaphthalen-1-yl)urea |

TABLE 2
WO 00/43384

| Ex- | |
|---|---|
| am- | |
| ple | |
| Num- | |
| ber | Compound Name |
| 5 | 1-[5-tert-butyl-2-p-tolyl-2H-pyrazol-3-yl]-3-[4-(2-morpholin-4- |
| | ylethoxy)naphthalen-1-yl]-urea |
| 6 | 1-[5-tert-butyl-2-p-tolyl-2H-pyrazol-3-yl]-3-[4-(2-(cis-2,6- |
| | dimethylmorpholin-4-yl)ethoxy)naphthalen-1-yl]-urea |
| 7 | 1-[5-tert-butyl-2-p-tolyl-2H-pyrazol-3-yl]-3-[4-(2-(trans-2,6- |
| | dimethylmorpholin-4-yl)ethoxy)naphthalen-1-yl]-urea |
| 8 | 1-[5-tert-butyl-2-p-tolyl-2H-pyrazol-3-yl]-3-[4-(2-(2- |
| | (methoxymethyl)morpholin-4-yl)ethoxy)naphthalen-1-yl]-urea |
| 9 | 1-[5-tert-butyl-2-p-tolyl-2H-pyrazol-3-yl]-3-[4-(2-(morpholin- |
| | 4-yl)-2-oxoethoxy)naphthalen-1-yl]-urea |
| 10 | 1-[5-tert-butyl-2-p-tolyl-2H-pyrazol-3-yl]-3-[4-(2-(morpholin- |
| | 4-yl)-2-methylethoxy)naphthalen-1-yl]-urea |
| 11 | 1-[5-tert-butyl-2-p-tolyl-2H-pyrazol-3-yl]-3-[4-(2-(morpholin- |
| | 4-yl)-1-methylethoxy)naphthalen-1-yl]-urea |
| 12 | 1-[5-tert-butyl-2-p-tolyl-2H-pyrazol-3-yl]-3-[4-(2-thiomorpholin-4- |
| | ylethoxy)naphthalen-1-yl]-urea |
| 13 | 1-[5-tert-butyl-2-p-tolyl-2H-pyrazol-3-yl]-3-[4-(2-(1- |
| | oxothiomorpholin-4-yl)ethoxy)naphthalen-1-yl]-urea |
| 14 | 1-[5-tert-butyl-2-p-tolyl-2H-pyrazol-3-yl]-3-[4-(2-morpholin-4-yl- |
| | ethoxy)-3-methylnaphthalen-1-yl]-urea |
| 15 | 1-[5-tert-butyl-2-p-tolyl-2H-pyrazol-3-yl]-3-[4-(2-(morpholin-4-yl- |
| | carbonyloxo)ethoxy)naphthalen-1-yl]-urea |
| 16 | 1-[5-tert-butyl-2-p-tolyl-2H-pyrazol-3-yl]-3-[4-(2- |
| | (tetrahydropyran-4-yl)ethoxy)naphthalen-1-yl]-urea |
| 17 | 1-[5-tert-butyl-2-p-tolyl-2H-pyrazol-3-yl]-3-[4-(2-(1-oxo- |
| | tetrahydrothiophen-3-yl)ethoxy)naphthalen-1-yl]-urea |
| 18 | 1-[5-tert-butyl-2-p-tolyl-2H-pyrazol-3-yl]-3-[4-(3-morpholin-4-yl- |
| | propyl)naphthalen-1-yl]-urea |
| 19 | 1-[5-tert-butyl-2-p-tolyl-2H-pyrazol-3-yl]-3-[4-(3-morpholin-4-yl- |
| | methyl)naphthalen-1-yl]-urea |
| 20 | 1-[5-tert-butyl-2-p-tolyl-2H-pyrazol-3-yl]-3-[4-(2-pyridin-4-yl- |
| | ethyl)naphthalen-1-yl]-urea |
| 21 | 1-[5-tert-butyl-2-p-tolyl-2H-pyrazol-3-yl]-3-[4-(3-(morpholin-4- |
| | yl)propyn-1-yl)naphthalen-1-yl]-urea |
| 22 | 1-[5-tert-butyl-2-p-tolyl-2H-pyrazol-3-yl]-3-[4-(3- |
| | (tetrahydropyran-2-yl-oxy)propyn-1-yl)naphthalen- |
| | 1-yl]-urea |
| 23 | 1-[5-tert-butyl-2-p-tolyl-2H-pyrazol-3-yl]-3-[4-(3- |
| | (tetrahydropyran-2-yl-oxy)butyn-1-yl)naphthalen- |
| | 1-yl]-urea |
| 24 | 1-[5-tert-butyl-2-p-tolyl-2H-pyrazol-3-yl]-3-[4-(3-(piperidin-1- |
| | yl)propyn-1-yl)naphthalen-1-yl]-urea |
| 25 | 1-[5-tert-butyl-2-p-tolyl-2H-pyrazol-3-yl]-3-[4-(3-(2- |
| | methoxymethylmorpholin-4-yl)propyn-1-yl)naphthalen-1-yl]-urea |
| 26 | 1-[5-tert-butyl-2-p-tolyl-2H-pyrazol-3-yl]-3-[4-(pyridin-4-yl- |
| | methoxy)naphthalen-1-yl]-urea |
| 27 | 1-[5-tert-butyl-2-p-tolyl-2H-pyrazol-3-yl]-3-[4-(2-pyridin-4-yl- |
| | ethoxy)naphthalen-1-yl]-urea |
| 28 | 1-[5-tert-butyl-2-p-tolyl-2H-pyrazol-3-yl]-3-[4-(3-pyridin-4-yl- |
| | propoxy)naphthalen-1-yl]-urea |
| 29 | 1-[5-tert-butyl-2-p-tolyl-2H-pyrazol-3-yl]-3-[4-(2-imidazol-1-yl- |
| | ethoxy)naphthalen-1-yl]-urea |
| 30 | 1-[5-tert-butyl-2-p-tolyl-2H-pyrazol-3-yl]-3-[4-(2-(3,4- |
| | dimethoxyphenyl)-ethoxy)naphthalen-1-yl]-urea |
| 31 | 1-[5-tert-butyl-2-p-tolyl-2H-pyrazol-3-yl]-3-[4-(pyridin-4-yl- |
| | methylamino)naphthalen-1-yl]-urea |
| 32 | 1-[5-isopropyl-2-phenyl-2H-pyrazol-3-yl]-3-[4-(2-morpholin-4-yl- |
| | ethoxy)naphthalen-1-yl]-urea |
| 33 | 1-[5-cyclohexyl-2-phenyl-2H-pyrazol-3-yl]-3-[4-(2-morpholin-4-yl- |
| | ethoxy)naphthalen-1-yl]-urea |
| 34 | 1-[5-(2,2,2-trifluoroethyl)-2-phenyl-2H-pyrazol-3-yl]-3-[4-(2- |
| | morpholin-4-yl-ethoxy)naphthalen-1-yl]-urea |
| 35 | 1-[5-(1-methylcycloprop-1-yl)-2-phenyl-2H-pyrazol-3-yl]-3-[4-(2- |
| | morpholin-4-yl-ethoxy)naphthalen-1-yl]-urea |
| 36 | 1-[5-(1-methylcyclohex-1-yl)-2-phenyl-2H-pyrazol-3-yl]-3-[4-(2- |
| | morpholin-4-yl-ethoxy)naphthalen-1-yl]-urea |
| 37 | 1-[5-tert-butyl-2-methyl-2H-pyrazol-3-yl[-3-[4-(2-morpholin-4-yl- |
| | ethoxy)naphthalen-1-yl]-urea |
| 38 | 1-[5-tert-butyl-2-(4-chlorophenyl)-2H-pyrazol-3-yl]-3-[4-(2- |
| | morpholin-4-yl-ethoxy)naphthalen-1-yl]-urea |
| 39 | 1-[5-tert-butyl-2-butyl-2H-pyrazol-3-yl]-3-[4-(2-morpholin-4-yl- |
| | ethoxy)naphthalen-1-yl]-urea |
| 40 | 1-[5-tert-butyl-2-(4-methyl-3-carbamylphenyl)-2H-pyrazol-3-yl]-3- |
| | [4-(2-morpholin-4-yl-ethoxy)naphthalen-1-yl]-urea |
| 41 | 1-[5-tert-butyl-2-(4-methyl-3-(morpholin-4-yl)methylphenyl)-2H- |
| | pyrazol-3-yl]-3-[4-(2-morpholin-4-yl-ethoxy)naphthalen-1-yl]-urea |
| 42 | 1-[5-tert-butyl-2-(4-methyl-3-dimethylaminomethylphenyl)-2H- |
| | pyrazol-3-yl]-3-[4-(2-morpholin-4-yl-ethoxy)naphthalen-1-yl]-urea |
| 43 | 1-[5-tert-butyl-2-(3-dimethylaminomethylphenyl)-2H-pyrazol- |
| | 3-yl]-3-[4-(2-morpholin-4-yl-ethoxy)naphthalen-1-yl]-urea |
| 44 | 1-[5-tert-butyl-2-(2-chloropyridin-5-yl)-2H-pyrazol-3-yl]-3-[4-(2- |
| | morpholin-4-yl-ethoxy)naphthalen-1-yl]-urea |
| 45 | 1-[5-tert-butyl-2-(2-methylpyridin-5-yl)-2H-pyrazol-3-yl]-3-[4-(2- |
| | morpholin-4-yl-ethoxy)naphthalen-1-yl]-urea |
| 46 | 1-[5-tert-butyl-2-(2-methoxypyridin-5-yl)-2H-pyrazol-3-yl]-3-[4-(2- |
| | morpholin-4-yl-ethoxy)naphthalen-1-yl]-urea |
| 47 | 1-[5-tert-butyl-2-(pyridin-3-yl)-2H-pyrazol-3-yl]-3-[4-(2- |
| | morpholin-4-yl-ethoxy)naphthalen-1-yl]-urea |
| 48 | 1-[5-tert-butyl-2-(2-methylpyridin-5-yl)-2H-pyrazol-3-yl]-3-[4-(2- |
| | pyridin-4-yl-ethoxy)naphthalen-1-yl]-urea |
| 49 | 1-[5-tert-butyl-2-(2-methylpyridin-5-yl)-2H-pyrazol-3-yl]-3-[4-(2- |
| | (trans-2,6-dimethylmorpholin-4-yl)ethoxy)naphthalen-1-yl]-urea |
| 50 | 1-[5-tert-butyl-2-(2-methylpyridin-5-yl)-2H-pyrazol-3-yl]-3-[4-(3- |
| | morpholin-4-yl-propyn-1-yl)naphthalen-1-yl]-urea |
| 51 | 1-[5-(2-hydroxy-1,1-dimethyl-ethyl)-2-p-tolyl-2H-pyrazol-3- |
| | yl]-3-[4-(2-morpholin-4-yl-ethoxy)naphthalen-1-yl]-urea |
| 52 | 1-[5-tert-butyl-2-(3-hydroxy-4-methyl-phenyl)-2H-pyrazol-3- |
| | yl]-3-[4-(2-morpholin-4-yl-ethoxy)naphthalen-1-yl]-urea |
| 53 | 1-[5-tert-butyl-2-(4-hydroxymethyl-phenyl)-2H-pyrazol-3- |
| | yl]-3-[4-(2-morpholin-4-yl-ethoxy)naphthalen-1-yl]-urea |
| 54 | 1-[5-tert-butyl-2-p-tolyl-2H-pyrazol-3-yl]-3-{4-[2-(3-oxo- |
| | morpholin-4-yl)-ethoxy]naphthalen-1-yl}-urea |
| 55 | 1-[5-tert-butyl-2-p-tolyl-2H-pyrazol-3-yl]-3-{4-[2-(4-hydroxy- |
| | morpholin-4-yl)-ethoxy]naphthalen-1-yl}-urea |
| 56 | 1-[5-(2-hydroxy-1,1-dimethyl-ethyl)-2-(6-methyl-pyridin-3-yl)-2H- |
| | pyrazol-3-yl]-3-[4-(2-morpholin-4-yl-ethoxy)naphthalen-1-yl]-urea |
| 57 | 1-[5-tert-butyl-2-(1-hydroxy-6-methyl-pyridin-3-yl)-2H-pyrazol-3- |
| | yl]-3-[4-(2-morpholin-4-yl-ethoxy)naphthalen-1-yl]-urea |
| 58 | 1-[5-tert-butyl-2-(6-methyl-pyridin-3-yl)-2H-pyrazol-3-yl]-3-{4-[2- |
| | (4-hydroxy-morpholin-4-yl)-ethoxy]naphthalen-1-yl}-urea |
| 59 | 1-[5-(2-hydroxy-1,1-dimethyl-ethyl)-2-(6-methyl-pyridin-3-yl)-2H- |
| | pyrazol-3-yl]-3-[4-(2-pyridin-4-yl-ethoxy)naphthalen-1-yl]-urea |
| 60 | 1-[5-tert-butyl-2-(6-methyl-pyridin-3-yl)-2H-pyrazol-3-yl]-3-[4-(2- |
| | hydroxy-2-pyridin-4-yl-ethoxy)naphthalen-1-yl]-urea |
| 61 | 1-[5-tert-butyl-2-(6-methyl-pyridin-3-yl)-2H-pyrazol-3-yl]-3-{4-[2- |
| | (1-hydroxy-2-pyridin-4-yl)-ethoxy]naphthalen-1-yl}-urea |
| 62 | 1-[5-(2-hydroxy-1,1-dimethyl-ethyl)-2-p-tolyl-2H-pyrazol-3- |
| | yl]-3-{4-[2-(1-oxo-thiomorpholin-4-yl)- |
| | ethoxy]naphthalen-1-yl}-urea |
| 63 | 1-[5-tert-butyl-2-(4-hydroxymethyl-phenyl)-2H-pyrazol-3-yl]-3-{4- |
| | [2-(1-oxo-thiomorpholin-4-yl)-ethoxy]naphthalen-1-yl}-urea |
| 64 | 1-[5-tert-butyl-2-p-tolyl-2H-pyrazol-3-yl]-3-{4-[2-(1,3-dioxo- |
| | thiomorpholin-4-yl)-ethoxy]naphthalen-1-yl}-urea |
| 65 | 1-[5-(2-hydroxy-1,1-dimethyl-ethyl)-2-methyl-2H-pyrazol-3- |
| | yl]-3-[4-(2-morpholin-4-yl-ethoxy)-naphthalen-1-yl]-urea |
| 66 | 1-[5-tert-butyl-2-methyl-2H-pyrazol-3-yl]-3-{4-[2-(4-hydroxy- |
| | morpholin-4-yl)-ethoxy]-naphthalen-1-yl}-urea |

TABLE 3
WO 00/55139

| Ex- | |
|---|---|
| am- | |
| ple | |
| Num- | |
| ber | Compound Name |
| 67 | 1-[5-tert-butyl-2-p-tolyl-2H-pyrazolyl-3-yl]-3-[4-(4-(morpholin-4- |
| | yl-methyl)phenyl)naphthalen-1-yl]urea |
| 68 | 1-[5-tert-butyl-2-p-tolyl-2H-pyrazolyl-3-yl]-3-[4-(4-(2- |
| | (morpholin-4-yl)ethyl)phenyl)naphthalen-1-yl]urea |
| 69 | 1-[5-tert-butyl-2-p-tolyl-2H-pyrazolyl-3-yl]-3-[4-(3-(morpholin-4- |
| | yl-methyl)phenyl)naphthalen-1-yl]urea |
| 70 | 1-[5-tert-butyl-2-p-tolyl-2H-pyrazolyl-3-yl]-3-[4-(6-(morpholin-4- |
| | ylmethyl-pyridin-3-yl)naphthalen-1-yl]urea |
| 71 | 1-[5-tert-butyl-2-p-tolyl-2H-pyrazolyl-3-yl]-3-[4-(5-(morpholin-4- |
| | ylmethyl-pyridin-2-yl)naphthalen-1-yl]urea |
| 72 | 1-[5-tert-butyl-2-p-tolyl-2H-pyrazolyl-3-yl]-3-[4-(5-(morpholin-4- |
| | ylmethyl-fur-2-yl)naphthalen-1-yl]urea |
| 73 | 1-[5-tert-butyl-2-(6-methyl-pyridin-3-yl)-2H-pyrazolyl-3-yl]-3- |
| | [4-(6-(morpholin-4-ylmethyl-pyridin-3-yl)naphthalen-1- |
| | yl]urea |
| 74 | 1-[5-tert-butyl-2-methyl-2H-pyrazolyl-3-yl]-3-[4-(6-morpholin-4- |
| | ylmethyl-pyridin-3-yl)naphthalen-1-yl]urea |
| 75 | 1-[5-tert-butyl-2-p-tolyl-2H-pyrazolyl-3-yl]-3-[4-(5-(morpholin- |
| | 4-yl-methyl)pyridin-2-yl)naphthalen-1-yl]urea |
| 76 | 1-[5-tert-butyl-2-p-tolyl-2H-pyrazolyl-3-yl]-3-[4-(6-(morpholin- |
| | 4-yl-methyl)pyridin-3-yl)naphthalen-1-yl]urea |
| 77 | 1-[5-tert-butyl-2-(6-methyl-pyridin-3-yl)-2H-pyrazolyl-3-yl]-3- |
| | [4-(3-(2-(pyridin-2-yl)ethylamino)cyclohexenyl)- |
| | naphthalen-1-yl]urea |
| 78 | 1-[5-tert-butyl-2-p-tolyl-2H-pyrazolyl-3-yl]-3-[4-(4-(pyridin-3-yl- |
| | methylaminomethyl)phenyl)naphthalen-1-yl]urea |
| 79 | 1-[5-tert-butyl-2-(6-methyl-pyridin-3-yl)-2H-pyrazolyl-3-yl]-3- |
| | [4-(4-(morpholin-4-yl-methyl)phenyl)naphthalen-1-yl]urea |
| 80 | 1-[5-tert-butyl-2-(6-methyl-pyridin-3-yl)-2H-pyrazolyl-3-yl]-3- |
| | [4-(6-(4-(hydroxybutylamino)pyridin-3-yl)-naphthalen- |
| | 1-yl]urea |
| 81 | 1-[5-tert-butyl-2-(4-methyl-3-carbamylphenyl)-2H-pyrazolyl-3- |
| | yl]-3-[4-(6-(morpholin-4-yl-methyl)pyridin-3-yl)naphthalen- |
| | 1-yl]urea |
| 82 | 1-[5-tert-butyl-2-(6-methyl-pyridin-3-yl)-2H-pyrazolyl-3-yl]-3- |
| | [4-(4-(3-hydroxypiuperidin-1-yl-methyl)phenyl)naphthalen- |
| | 1-yl]urea |
| 83 | 1-[5-tert-butyl-2-(6-methyl-pyridin-3-yl)-2H-pyrazolyl-3-yl]-3- |
| | [4-(4-(4-hydroxymorpholin-4-yl-methyl)phenyl)naphthalen- |
| | 1-yl]urea |
| 84 | 1-[5-tert-butyl-2-(6-methyl-pyridin-3-yl)-2H-pyrazolyl-3-yl]-3- |
| | [4-(3-(morpholin-4-yl-methyl)cyclohexenyl)naphthalen- |
| | 1-yl]urea |
| 85 | 1-[5-tert-butyl-2-(6-methyl-pyridin-3-yl)-2H-pyrazolyl-3-yl]-3- |
| | [4-(4-(tetrahydrofuran-3-yl-methyl)-3- |
| | hydroxyphenyl)naphthalen-1-yl]urea |
| 86 | 1-[5-tert-butyl-2-(6-methyl-pyridin-3-yl)-2H-pyrazolyl-3-yl]-3- |
| | [4-(4-(N,N-di-(2- |
| | methoxyethyl)aminomethyl)phenyl)naphthalen-1-yl]urea |
| 87 | 1-[5-tert-butyl-2-(6-methyl-pyridin-3-yl)-2H-pyrazolyl-3-yl]-3- |
| | [4-(6-(cyanopropoxy)pyridin-3-yl)naphthalen-1-yl]urea |
| 88 | 1-[5-tert-butyl-2-(6-methyl-pyridin-3-yl)-2H-pyrazolyl-3-yl]-3- |
| | [4-(4-morpholin-4-yl-methyl-piperidinyl)naphthalen- |
| | 1-yl]urea |
| 89 | 1-[5-tert-butyl-2-(6-methyl-pyridin-3-yl)-2H-pyrazolyl-3-yl]-3- |
| | [4-(4-(N,N-di-(2- |
| | cyanoethyl)aminomethyl)phenyl)naphthalen-1-yl]urea |
| 90 | 1-[5-tert-butyl-2-(6-methyl-pyridin-3-yl)-2H-pyrazolyl-3-yl]-3- |
| | [4-(4-(furan-2-yl-methyl)-3-hydroxyphenyl)naphthalen- |
| | 1-yl]urea |
| 91 | 1-[5-tert-butyl-2-(6-methyl-pyridin-3-yl)-2H-pyrazolyl-3-yl]-3- |
| | [4-(4-(thiomorpholin-4-yl-methyl)phenyl)naphthalen- |
| | 1-yl]urea |
| 92 | 1-[5-tert-butyl-2-(6-methyl-pyridin-3-yl)-2H-pyrazolyl-3-yl]-3- |
| | [4-(4-(3-carboxamidopiperidin-1-yl- |
| | methyl)phenyl)naphthalen-1-yl]urea |
| 93 | 1-[5-tert-butyl-2-(6-methyl-pyridin-3-yl)-2H-pyrazolyl-3-yl]-3- |
| | [4-(4-(2-methyl-3-oxo-piperzin-1-yl- |
| | methyl)phenyl)naphthalen-1-yl]urea |
| 94 | 1-[5-tert-butyl-2-(2-methylpyrimidin-5-yl)-2H-pyrazolyl-3-yl]-3-[4- |
| | (6-(morpholin-4-yl-methyl)pyridin-3-yl)naphthalen-1-yl]urea |
| 95 | 1-[5-tert-butyl-2-(6-methyl-pyridin-3-yl)-2H-pyrazolyl-3- |
| | yl]-3-[4-(6-(4-hydroxybutyloxy)pyridin-3- |
| | yl)naphthalen-1-yl]urea |
| 96 | 1-[3-tert-butyl-1′H-[1,4′]bipyrazol-5-yl]-3-[4-(6-(morpholin-4-yl- |
| | methyl)pyridin-3-yl)naphthalen-1-yl]urea |
| 97 | 1-[5-tert-butyl-2-2H-pyrazolyl-3-yl]-3-[4-(6- |
| | (tetrahydrothiopyran-4-yl-amino)pyridin-3- |
| | yl)naphthalen-1-yl]urea |
| 98 | 1-[5-tert-butyl-2-(2-cyanoethyl)-2H-pyrazolyl-3-yl]-3-[4-(6- |
| | (morpholin-4-yl-methyl)pyridin-3-yl)naphthalen-1-yl]urea |
| 99 | 1-[5-tert-butyl-2-(6-methyl-pyridin-3-yl)-2H-pyrazolyl-3-yl]-3- |
| | [4-(6-(2,6-dim ethylmorpholin-4-yl-methyl)pyridin-3- |
| | yl)naphthalen-1-yl]urea |
| 100 | 1-[5-tert-butyl-2-(2-methoxypyridin-5-yl)-2H-pyrazolyl-3-yl]-3- |
| | [4-(6-(morpholin-4-yl-methyl)pyridin-3-yl)naphthalen- |
| | 1-yl]urea |
| 101 | 1-[5-tert-butyl-2-(2-aminopyridin-5-yl)-2H-pyrazolyl-3-yl]-3-[4-(6- |
| | (morpholin-4-yl-methyl)pyridin-3-yl)naphthalen-1-yl]urea |
| 102 | 1-[5-tert-butyl-2-(6-methyl-pyridin-3-yl)-2H-pyrazolyl-3-yl]-3- |
| | [4-(6-(morpholin-4-yl-4-carbonyl)pyridin-3-yl)naphthalen- |
| | 1-yl]urea |
| 103 | 1-[5-tert-butyl-2-(6-methyl-pyridin-3-yl)-2H-pyrazolyl-3-yl]-3- |
| | [4-(6-(2-oxa-5-aza-bicyclo[2.2.1]hept-5-yl-methyl)pyridin- |
| | 3-yl)naphthalen-1-yl]urea |
| 104 | 1-[5-tert-butyl-2-(6-methyl-pyridin-3-yl)-2H-pyrazolyl-3-yl]-3- |
| | [4-(4-(N-(2-cyanoethyl)-N-(pyridin-3- |
| | ylmethyl)aminomethyl)phenyl)naphthalen-1-yl]urea |
| 105 | 1-[5-tert-butyl-2-(6-methyl-pyridin-3-yl)-2H-pyrazolyl-3-yl]-3- |
| | [4-(4-(N-(2-cyanoethyl)-N-(tetrahydrofuran-2-yl- |
| | methyl)aminomethyl)phenyl)naphthalen-1-yl]urea |
| 106 | 1-[5-tert-butyl-2-(6-methyl-pyridin-3-yl)-2H-pyrazolyl-3-yl]-3- |
| | [4-(6-(morpholin-4-yl-methyl)-4-methoxypyridin-3- |
| | yl)naphthalen-1-yl]urea |
| 107 | 1-[5-tert-butyl-2-(6-methyl-pyridin-3-yl)-2H-pyrazolyl-3-yl]-3- |
| | [4-(6-(1-morpholin-4-yl-propyl)pyridin-3-yl)naphthalen-1- |
| | yl]urea |
| 108 | 1-[3-tert-butyl-1′-methyl-1′H-[1,4′]bipyrazol-5-yl]-3-[4-(6- |
| | (morpholin-4-yl-methyl)pyridin-3-yl)naphthalen-1-yl]urea |
| 109 | 1-[5-tert-butyl-2-(6-methyl-pyridin-3-yl)-2H-pyrazolyl-3-yl]-3- |
| | [4-(6-(1-oxo-tetrahydrothiopyran-4-yl-amino)pyridin-3- |
| | yl)naphthalen-1-yl]urea |
| 110 | 1-[5-tert-butyl-2-(6-methyl-pyridin-3-yl)-2H-pyrazolyl-3-yl]-3- |
| | [4-(6-(tetrahydropyran-4-yl-amino)pyridin-3-yl)naphthalen- |
| | 1-yl]urea |
| 111 | 1-[5-tert-butyl-2-(6-methyl-pyridin-3-yl)-2H-pyrazolyl-3-yl]-3- |
| | [4-(5-(tetrahydrothiopyran-4-yl-aminbo)pyrazin-2- |
| | yl)naphthalen-1-yl]urea |
| 112 | 1-[5-tert-butyl-2-(6-methyl-pyridin-3-yl)-2H-pyrazolyl-3-yl]-3- |
| | [4-(6-(metrhylcarbonylamino)pyridin-3-yl)naphthalen- |
| | 1-yl]urea |
| 113 | 1-[5-tert-butyl-1′-(3-methylsulfanylpropyl)-1′H-[1,4′]bipyrazol-5- |
| | yl]-3-[4-(6-(morpholin-4-yl-methyl)pyridin-3-yl)naphthalen- |
| | 1-yl]urea |
| 114 | 1-[5-tert-butyl-2-(2-methylpyrimidin-5-yl)-2H-pyrazolyl-3-yl]-3-[4- |
| | (6-(1-oxo-thiomorpholin-4-yl-methyl)pyridin-3-yl)naphthalen-1- |
| | yl]urea |
| 115 | 1-[5-tert-butyl-2-(2-methylpyrimidin-5-yl)-2H-pyrazolyl-3-yl]-3-[4- |
| | (6-(tetrahydropyran-4-ylamino)pyridin-3-yl)naphthalen-1-yl]urea |
| 116 | 1-[5-tert-butyl-2-(2-methylthiopyrimidin-5-yl)-2H-pyrazolyl-3- |
| | yl]-3-[4-(6-(morpholin-4-yl-methyl)pyridin-3-yl)naphthalen- |
| | 1-yl]urea |
| 117 | 1-[5-tert-butyl-2-(2-aminopyrimidin-5-yl)-2H-pyrazolyl-3-yl]-3-[4- |
| | (6-(morpholin-4-yl-methyl)pyridin-3-yl)naphthalen-1-yl]urea |
| 118 | 1-[3-tert-butyl-1′-methyl-1′H-[1,4;]bipyrazol-5-yl]-3-[4-(6- |
| | (morpholin-4-yl-methyl)phenyl)naphthalen-1-yl]urea |
| 119 | 1-[5-tert-butyl-2-p-tolyl-2H-pyrazolyl-3-yl]-3-[4-(6-(1-oxo- |
| | tetrahydrothiopyran-4-yl-amino)pyridin-3-yl)naphthalen-1-yl]urea |
| 120 | 1-[5-tert-butyl-2-p-tolyl-2H-pyrazolyl-3-yl]-3-[4-(6-(thiomorpholin- |
| | 4-yl-methyl)pyridin-3-yl)naphthalen-1-yl]urea |
| 121 | 1-[5-tert-butyl-2-p-tolyl-2H-pyrazolyl-3-yl]-3-[4-(2-(morpholin- |
| | 4-yl-carbonyl)pyrimidin-5-yl)naphthalen-1-yl]urea |
| 122 | 1-[5-tert-butyl-2-p-tolyl-2H-pyrazolyl-3-yl]-3-[4-(4-(morpholin- |
| | 4-yl-methyl)pyrimidin-5-yl)naphthalen-1-yl]urea |
| 123 | 1-[5-tert-butyl-2-p-tolyl-2H-pyrazolyl-3-yl]-3-[4-(6-(1-oxo- |
| | thiomorpholin-4-yl-methyl)pyridin-3-yl)naphthalen-1-yl]urea |
| 124 | 1-[5-tert-butyl-2-(2-methylpyrimidin-5-yl)-2H-pyrazolyl-3-yl]-3-[4- |
| | (2-(morpholin-4-yl-methyl)pyrimidin-5-yl)naphthalen-1-yl]urea |
| 125 | 1-[2-tert-butyl-5-methyl-pyridin-4-yl]-3-[4-(6-(morpholin-4-yl- |
| | methyl-pyridin-3-yl)naphthalen-1-yl]urea |
| 126 | 1-[3-tert-butyl-phenyl]-3-[4-(6-(morpholin-4-yl-methyl)pyridin-3- |
| | yl)naphthalen-1-yl]urea |
| 127 | 1-[4-methyl-biphenyl-3-yl]-3-[4-(6-(morpholin-4-yl-methyl- |
| | pyridin-3-yl)naphthalen-1-yl]urea |
| 128 | 1-[4-tert-butyl-biphenyl-2-yl]-3-[4-(6-morpholin-4-yl-methyl- |
| | pyridin-3-yl)naphthalen-1-yl]urea |
| 129 | 1-[5-isopropyl-2-methyl-phenyl]-3-[4-(6-morpholin-4-yl-methyl- |
| | pyridin-3-yl)naphthalen-1-yl]urea |
| 130 | 1-[5-sec-butyl-2-methoxy-phenyl]-3-[4-(6-morpholin-4-yl-methyl- |
| | pyridin-3-yl)naphthalen-1-yl]urea |
| 131 | 1-[5-tert-butyl-2-methoxymethyl-phenyl]-3-[4-(6-(morpholin-4-yl- |
| | methyl-pyridin-3-yl)naphthalen-1-yl]urea |
| 132 | 1-[5-tert-butyl-2-methoxy-phenyl]-3-[4-(6-(morpholin-4-yl-methyl- |
| | pyridin-3-yl)naphthalen-1-yl]urea |
| 133 | 1-[5-tert-butyl-2-mewthyl-phenyl]-3-{6-[(3-(methoxy-propyl)- |
| | methyl-amino]-pyridin-3-yl}naphthalen-1-yl]urea |
| 134 | 1-[5-tert-butyl-2-methyl-phenyl]-3-[4-(6-morpholin-4-yl-methyl- |
| | pyridin-3-yl)naphthalen-1-yl]urea |
| 135 | 1-[5-tert-butyl-2-methyl-pyridin-3-yl]-3-[4-(6-morpholin-4-yl- |
| | methyl-pyridin-3-yl)naphthalen-1-yl]urea |
| 136 | 1-[5-(1,1-dimethylpropyl)-2-methoxy-phenyl]-3-[4-(6-morpholin-4- |
| | yl-methyl-pyridin-3-yl)naphthalen-1-yl]urea |
| 137 | 1-[5-tert-butyl-2-(1H-pyrazol-4-yl)-phenyl]-3-[4-(6-morpholin-4-yl- |
| | methyl-pyridin-3-yl)naphthalen-1-yl]urea |
| 138 | 1-[5-tert-butyl-2-(2-methyl-pyrimidin-5-yl)-phenyl]-3-[4-(6- |
| | morpholin-4-yl-methyl-pyridin-3-yl)naphthalen-1-yl]urea |
| 139 | 1-[5-tert-butyl-2-(3-hydroxy-propyl)-phenyl]-3-[4-(6-morpholin- |
| | 4-yl-methyl-pyridin-3-yl)naphthalen-1-yl]urea |
| 140 | 1-[5-tert-butyl-2-(morpholine-4-carbonyl)-phenyl]-3-[4-(6- |
| | morpholin-4-yl-methyl-pyridin-3-yl)naphthalen-1-yl]urea |
| 141 | 1-[5-tert-butyl-2-methoxy-3-{3-[4-(6-morpholin-4-ylmethyl- |
| | pyridin-3-yl)-naphthalen-1-yl]-ureido}-phenyl)-acetamide |
| 142 | 1-[3-methyl-naphthalen-2-yl]-3-[4-(6-morpholin-4-yl-methyl- |
| | pyridin-3-yl)naphthalen-1-yl]urea |
| 143 | 1-[5-tert-butyl-2-methoxy-3-{3-[4-(6-morpholin-4-ylmethyl- |
| | pyridin-3-yl)-naphthalen-1-yl]-urido}-phenyl)-acetamide |
| 144 | 1-[5-tert-butyl-3-(2,3-dihydroxy-propyl)-2-hydroxy-phenyl]- |
| | 3-[4-(6-morpholin-4-yl-methyl-pyridin-3-yl)naphthalen-1- |
| | yl]urea |
| 145 | 1-[2,3-dimethyl-1H-indol-5-yl]-3-[4-(6-morpholin-4-yl-methyl- |
| | pyridin-3-yl)naphthalen-1-yl]urea |
| 146 | 1-{5-tert-butyl-2-methyl-3-[3-(tetrahydro-pyran-2-yloxy)-prop-1- |
| | ynyl]-phenyl}-3-[4-(6-morpholin-4-yl-methyl-pyridin-3- |
| | yl)naphthalen-1-yl]urea |
| 147 | 1-[2-methoxy-5-trifluoromethyl-phenyl]-3-[4-(6-morpholin-4-yl- |
| | methyl-pyrimidin-3-yl)naphthalen-1-yl]urea |
| 148 | 1-[5-(2,2-dimethyl-propionyl)-2-methyl-phenyl]-3-[4-(6-morpholin- |
| | 4-yl-methyl-pyridin-3-yl)naphthalen-1-yl]urea |
| 149 | 1-[5-tert-butyl-3-(3-hydroxy-prop-1-ynyl)-2-methyl-phenyl]-3- |
| | [4-(6-morpholin-4-yl-methyl-pyridin-3-yl)naphthalen-1- |
| | yl]urea |
| 150 | 1-[5-tert-butyl-2-(3-hydroxy-propyn-1-ynyl)-phenyl]-3-[4-(6- |
| | morpholin-4-yl-methyl-pyridin-3-yl)naphthalen-1-yl]urea |
| 151 | 1-[5-tert-butyl-3-(2,2-dimethyl-[1,3]dioxolan-4-ylmethyl)-2- |
| | methoxy-phenyl]-3-[4-(6-morpholin-4-yl-methyl-pyridin-3- |
| | yl)naphthalen-1-yl]urea |
| 152 | 1-[5-tert-butyl-3-(2,3-dihydroxy-propyl)-2-methoxy-phenyl]-3- |
| | [4-(6-morpholin-4-yl-methyl-pyridin-3-yl)naphthalen-1- |
| | yl]urea |
| 153 | 1-[5-tert-butyoxy-2-methoxy-phenyl]-3-[4-(6-morpholin-4-yl- |
| | methyl-pyridin-3-yl)naphthalen-1-yl]urea |
| 154 | 1-[5-(1-cyano-cyclopropyl)-2-methoxy-phenyl]-3-[4-(6- |
| | morpholin-4-yl-methyl-pyridin-3-yl)naphthalen-1- |
| | yl]urea |
| 155 | 1-[5-tert-butyl-3-(2-diethylamino-ethyl)-2-methoxy-phenyl]-3- |
| | [4-(6-morpholin-4-yl-methyl-pyridin-3-yl)naphthalen-1- |
| | yl]urea |
| 156 | 1-[5-tert-butyl-2-methoxy-phenyl]-3-[4-(6-[1,3]dioxolan-2-yl- |
| | pyridin-3-yl)naphthalen-1-yl]urea |
| 157 | 1-[5-tert-butyl-2-pyrrolidin-1-yl-phenyl]-3-[4-(6-morpholin-4-yl- |
| | methyl-pyridin-3-yl)naphthalen-1-yl]urea |
| 158 | 1-[5-tert-butyl-2-dimethylamino-phenyl]-3-[4-(6-morpholin-4-yl- |
| | methyl-pyridin-3-yl)naphthalen-1-yl]urea |
| 159 | 1-[5-tert-butyl-2-propoxy-phenyl]-3-[4-(6-morpholin-4-yl-methyl- |
| | pyridin-3-yl)naphthalen-1-yl]urea |
| 160 | 1-[5-tert-butyl-2-methoxy-phenyl]-3-[4-(6-hydroxymethyl- |
| | pyridin-3-yl)naphthalen-1-yl]urea |
| 161 | 1-[5-tert-butyl-2-methoxy-phenyl]-3-{4-[6-(2,6-dimethyl- |
| | morpholin-4-yl-methyl)pyridin-3-yl]naphthalen-1- |
| | yl}urea |
| 162 | 1-[5-cyclohexyl-2-methoxy-phenyl]-3-[4-(6-morpholin-4-yl- |
| | methyl-pyridin-3-yl)naphthalen-1-yl]urea |
| 163 | 1-[2,4-dimethoxy-5-trifluoromethyl-phenyl]-3-[4-(6-morpholin- |
| | 4-yl-methyl-pyridin-3-yl)naphthalen-1-yl]urea |
| 164 | 1-[5-tert-butyl-2-methoxy-3-nitro-phenyl]-3-[4-(6-morpholin-4-yl- |
| | methyl-pyridin-3-yl)naphthalen-1-yl]urea |
| 165 | 1-[3-amino-5-tert-butyl-2-methoxy-phenyl]-3-[4-(6-methyl- |
| | pyridin-3-yl)naphthalen-1-yl]urea |
| 166 | N-acetyl-N-(5-tert-butyl-2-methoxy-3-{3-[4-(6-morpholin-4- |
| | ylmethyl-pyridin-3-yl)-naphthalen-1-yl]-ureido}-phenyl)-acetamide |
| 167 | 1-[6-tert-butyl-4-methyl-3-oxo-3,4-dihydro-2H- |
| | benzo[1,4]oxazin-8-yl]-3-[4-(6-morpholin-4- |
| | ylmethyl-pyridin-3-yl)naphthalen-1-yl]urea |
| 168 | 1-[5-tert-butyl-2-ethoxy-phenyl]-3-[4-(6-morpholin-4-yl-methyl- |
| | pyridin-3-yl)naphthalen-1-yl]urea |
| 169 | 1-[5-tert-butyl-2-isopropoxy-phenyl]-3-[4-(6-morpholin-4-yl- |
| | methyl-pyridin-3-yl)naphthalen-1-yl]urea |
| 170 | 1-[5-tert-butyl-2-imidazol-1-yl-phenyl]-3-[4-(6-morpholin-4-yl- |
| | methyl-pyridin-3-yl)naphthalen-1-yl]urea |
| 171 | 1-[5-tert-butyl-3-ethylamino-2-methoxy-phenyl]-3-[4-(6- |
| | morpholin-4-yl-methyl-pyridin-3-yl)naphthalen-1-yl]urea |
| 172 | 1-[5-tert-butyl-2-methoxy-3-{3-[4-(6-morpholin-4-ylmethyl- |
| | pyridin-3-yl)-naphthalen-1-yl]-ureido}-phenyl)- |
| | bis(methanesulfon)amide |
| 173 | 1-[5-tert-butyl-2-(1-methyl-1H-pyrazol-4-yl)-phenyl]-3-[4-(6- |
| | morpholin-4-yl-methyl-pyridin-3-yl)naphthalen-1-yl]urea |
| 174 | 1-[2-methanesulfinyl-5-trifluoromethyl-phenyl]-3-[4-(6- |
| | morpholin-4-yl-methyl-pyridin-3-yl)naphthalen-1- |
| | yl]urea |
| 175 | 1-[4-(6-{[bis(2-methoxy-ethyl)-amino]-methyl}-pyridin-3-yl- |
| | naphthalen-1-yl]-3-[5-tert-2-methoxy-phenyl]urea |
| 176 | N-[1-(5-{4-[3-(5-tert-butyl-2-methoxy-phenyl)-ureido]- |
| | naphthalen-1-yl}-pyridin-2-ylmethyl)-pyrrolidin-3- |
| | yl]-acetamide |
| 177 | 1-[1-acetyl-3,3-dimethyl-2,3-dihydro-1H-indol-5-yl]-3-[4-(6- |
| | morpholin-4-yl-methyl-pyridin-3-yl)naphthalen-1-yl]urea |
| 178 | 1-(5-tert-butyl-2-methoxy-3-{3-[4-(6-morpholin-4-ylmethyl- |
| | pyridin-3-yl)-naphthalen-1-yl]-ureido}-phenyl)- |
| | propionamide |
| 179 | 1-[5-tert-butyl-2-methyl-benzooxazol-7-yl]-3-[4-(6-morpholin-4-yl- |
| | methyl-pyridin-3-yl)naphthalen-1-yl]urea |
| 180 | 1-[3-trifluoromethanesulfonyl-phenyl]-3-[4-(6-morpholin-4-yl- |
| | methyl-pyridin-3-yl)naphthalen-1-yl]urea |
| 181 | N-(5-tert-butyl-2-methoxy-3-{3-{4-(6-morpholin-4-ylmethyl- |
| | pyridin-3-yl)-naphthalen-1-yl]-ureido}-phenyl)- |
| | isobutyramide |
| 182 | 2-(4-tert-butyl-2-{3-[4-(6-morpholin-4-ylmethyl-pyridin-3-yl)- |
| | naphthalen-1-yl]-ureido}-phenoxy)-acetamide |
| 183 | 1-[5-tert-butyl-2-oxo-2,3-dihydro-benzooxazol-7-yl]-3-[4-(6- |
| | morpholin-4-yl-methyl-pyridin-3-yl)naphthalen-1-yl]urea |
| 184 | 1-[5-tert-butyl-3-cyano-2-methoxy-phenyl]-3-[4-(6-morpholin-4-yl- |
| | methyl-pyridin-3-yl)naphthalen-1-yl]urea |
| 185 | 1-[5-tert-butyl-benzooxazol-7-yl]-3-[4-(6-morpholin-4-yl-methyl- |
| | pyridin-3-yl)naphthalen-1-yl]urea |
| 186 | 5-tert-butyl-2-methoxy-{3-[4-(6-morpholin-4-ylmethyl-pyridin- |
| | 3-yl)-naphthalen-1-yl]-ureido}-phenyl)- |
| | benzenesulfonamide |
| 187 | Ethanesulfonic acid (5-tert-butyl-2-methoxy-3-{3-[4-(6- |
| | morpholin-4-ylmethyl-pyridin-3-yl)-naphthalen-1- |
| | yl]-ure3ido}-phenyl)-amide |
| 188 | 1-[5-tert-butyl-2-methoxy-phenyl]-3-[4-(2-morpholin-4-yl-methyl- |
| | pyrimidin-5-yl)naphthalen-1-yl]urea |
| 189 | 1-[5-tert-butyl-2-methylsulfanyl-phenyl]-3-[4-(6-morpholin-4-yl- |
| | methylpyridin-3-yl)naphthalen-1-yl]urea |
| 190 | 1-[5-tert-butyl-2-methoxy-pyridin-3-yl]-3-[4-(6-morpholin-4-yl- |
| | methyl6-pyridin-3-yl)naphthalen-1-y1]urea |
| 191 | 2,2,2-trifluoroethanesulfonic acid (5-tert-butyl-2-methoxy-3- |
| | {3-[4-(6-morpholin-4-ylmethyl-pyridin-3-yl)- |
| | naphthalen-1-yl]-ureido}-phenyl)-amide |
| 192 | N-(5-{4-[3-(5-tert-butyl-2-methyl-phenyl)-ureido]-naphthalen-1- |
| | yl}-pyrazin-2-yl)-methanesulfonamide |
| 193 | 1-[5-tert-butyl-2-methoxy-phenyl]-3-[4-(6-{[bis-(2-cyano-ethyl)- |
| | amino]-methyl}-pyridin-3-yl)naphthalen-1-yl]urea |
| 194 | 1-[5-tert-butyl-2-methoxy-phenyl]-3-{4-[6-(4-methyl-piperazin-1- |
| | ylmethyl)pyridin-3-yl]naphthalen-1-yl}urea |
| 195 | 1-[5-tert-butyl-2-methoxy-phenyl]-3-[4-(6-thiomorpholin-4-yl- |
| | methyl-pyridin-3-yl)naphthalen-1-yl]urea |
| 196 | 1-[5-tert-butyl-2-methoxy-phenyl]-3-{4-[6-(2,6-dimethyl- |
| | piperidin-1-ylmethyl)-pyridin-3-yl]naphthalen-1-yl}urea |
| 197 | 1-[5-tert-butyl-2-methoxy-phenyl]-3-{4-[6-(1-oxo- |
| | tetrahydropyran-4-ylamino)-pyridin-3-yl]naphthalen-1- |
| | yl}urea |
| 198 | 1-[5-tert-butyl-2-methoxy-phenyl]-3-{4-[6-(tetrahydropyran-4- |
| | ylamino)-pyridin-3-yl]naphthalen-1-yl}urea |
| 199 | 1-[5-tert-butyl-2-methoxy-phenyl]-3-[4-[6-{[(2-cyano-ethyl)- |
| | tetrahydro-furan-2-ylmethyl)-amino]-methyl}-pyridin-3- |
| | yl)naphthalen-1-yl}urea |
| 200 | 1-[5-tert-butyl-2-methoxy-phenyl]-3-{4-[6-(2-methoxymethyl- |
| | morpholin-4-ylmethyl)-pyridin-3-yl]naphthalen-1-yl}urea |
| 201 | 1-[5-tert-butyl-2-methoxy-phenyl]-3-{4-[6-(2-methyl-3-oxo- |
| | piperazin-1-ylmethyl)-pyridin-3-yl]naphthalen-1-yl}urea |
| 202 | 1-(5-{4-[3-(5-tert-butyl-2-methoxy-phenyl)-ureido]-naphthalen- |
| | 1-yl}-pyridin-2-ylmethyl)-piperidine-3- |
| | carboxylic acid amide |
| 203 | 1-[5-tert-butyl-2-methoxy-phenyl]-3-{4-[6-(1-oxo-thiomorpholin-4- |
| | ylmethyl)-pyridin-3-yl]naphthalen-1-yl}urea |
| 204 | 1-(3,3-dimethyl-2-oxo-2,3-dihydro-1H-indol-5-yl)-3-[4-(6- |
| | morpholin-4-ylmethyl-pyridin-3-yl)-naphthalen-1-yl]urea |
| 205 | 1-[5-tert-butyl-2-methoxy-phenyl]-3-{4-[6-(3-oxo-piperazin-1- |
| | ylmethyl)-pyridin-3-yl]naphthalen-1-yl}urea |
| 206 | 1-[5-tert-butyl-2-methoxy-phenyl]-3-(4-{6-[(tetrahydro-furan-3- |
| | ylamino)methyl]-pyridin-3-yl}naphthalen-1-yl}urea |
| 207 | 1-[5-tert-butyl-2-methoxy-phenyl]-3-[4-(6-{[(2-cyano-ethyl)- |
| | pyridin-3-ylmethyl-amino]-methyl}-pyridin-3-yl)naphthalen- |
| | 1-yl]urea |
| 208 | 1-[5-tert-butyl-2-methoxy-phenyl]-3-{4-[6-(2-oxa-5-aza- |
| | bicyclo[2.2.1]hept-5-ylmethyl)-pyridin-3-yl]naphthalen-1-yl}urea |
| 209 | 1-[5-tert-butyl-2-methoxy-phenyl]-3-{4-[6-(2,6-dimethyl- |
| | morpholin-4-ylmethyl)pyridin-3-yl]naphthalen-1- |
| | yl}urea |
| 210 | 1-[5-tert-butyl-2-methoxy-phenyl]-3-(4-{6-[4-(3-methoxy- |
| | phenyl)-1-piperazin-1-ylmethyl]-pyridin-3-yl}naphthalen- |
| | 1-yl)urea |
| 211 | 1-[5-tert-butyl-2-methoxy-phenyl]-3-{4-[6-(morpholine-4- |
| | carbonyl)-pyridin-3-yl]naphthalen-1-yl}urea |
| 212 | 1-[5-tert-butyl-2-methoxy-phenyl]-3-[4-(5-morpholin-4-ylmethyl- |
| | pyrazin-2-yl)-naphthalen-1-yl}urea |
| 213 | 1-[6-tert-butyl-3-oxo-3,4-dihydro-2H-benzo[1,4]oxazin-8-yl]-3- |
| | [4-(6-morpholin-4-ylmethyl-pyridin-3-yl) naphthalen- |
| | 1-yl]urea |
| 214 | 1-[3-amino-5-tert-butyl-2-methoxy-phenyl]-3-[4-(6-morpholin-4-yl- |
| | methyl-pyridin-3-yl)naphthalen-1-yl]urea |
| 215 | N-(5-{4-[3-(5-tert-butyl-2-methoxy-phenyl)-ureido]-naphthalen-1- |
| | yl}pyridin-2-yl)-acetamide |
| 216 | N-(5-tert-butyl-2-methoxy-3-{3-[4-(6-morpholin-4-ylmethyl- |
| | pyridin-3-yl)-naphthalen-1-y1]-ureido}-phenyl)- |
| | N-methyl-acetamide |
| 217 | N-(5-tert-butyl-2-methoxy-3-{3-[4-(6-morpholin-4-ylmethyl- |
| | pyridin-3-yl)-naphthalen-1-yl]-ureido}-phenyl)- |
| | 2,2,2-trifluoro-acetamide |
| 218 | 1-[5-tert-butyl-2-methoxy-phenyl]-3-{4-[6-(pyridin-3-yloxy)- |
| | pyridin-3-yl]naphthalen-1-yl}urea |
| 219 | [4-(6-morpholin-4-ylmethyl-pyridin-3-yl)-naphthalen-1-yl]- |
| | carbamic acid 3-tert-butyl-phenyl ester |
| 220 | N-(5-tert-butyl-2-methoxy-3-{3-[4-(6-morpholin-4-ylmethyl- |
| | pyridin-3-yl)-naphthalen-1-yl]-ureido}-phenyl)- |
| | methanesulfonamide |

TABLE 4
WO 00/55152

| Ex- | |
|---|---|
| am- | |
| ple | |
| Num- | |
| ber | Compound Name |
| 221 | 1-[2-carbomethoxy-5-tert-butyl-3-thienyl]-3-[4-(2-morpholin-4-yl- |
| | ethoxy)naphthalen-1-yl]urea |
| 222 | 1-[2-methylcarbamoyl-5-tert-butyl-3-thienyl]-3-[4-(2-morpholin- |
| | 4-yl-ethoxy)naphthalen-1-yl]urea |
| 223 | 1-[2-methylcarbamoyl-5-tert-butyl-3-thienyl]-3-[4-(2-(trans-2,6- |
| | dimethylmorpholin-4-yl)ethoxy)naphthalen-1-yl]urea |
| 224 | 1-[2-methylcarbamoyl-5-tert-butyl-3-thienyl]-3-[4-(2-(2- |
| | dimethylaminomethyldimethylmorpholin-4-yl)ethoxy) |
| | naphthalen-1-yl]urea |
| 225 | 1-[2-carbomethoxy-5-tert-butyl-3-thienyl]-3-[4-(2-(1- |
| | oxothiomorpholin-4-yl)ethoxy)naphthalen-1-yl]urea |
| 226 | 1-[2-methylcarbamoyl-5-tert-butyl-3-thienyl]-3-[4-(2-(1- |
| | oxothiomorpholin-4-yl)ethoxy)naphthalen-1-yl]urea |
| 227 | 1-[3-carbomethoxy-5-tert-butyl-2-thienyl]-3-[4-(2-(1- |
| | oxothiomorpholin-4-yl)ethoxy)naphthalen-1-yl]urea |
| 228 | 1-[2-carbomethoxy-5-tert-butyl-3-thienyl]-3-[4-(2- |
| | (tetrahydropyran-4-yl)ethoxy )naphthalen-1-yl]urea |
| 229 | 1-[2-methylcarbamoyl-5-tert-butyl-3-thienyl]-3-[4-(2- |
| | (tetrahydropyran-4-yl)ethoxy)naphthalen-1-yl]urea |
| 230 | 1-[3-carbomethoxy-5-tert-butyl-2-thienyl]-3-[4-(2- |
| | (tetrahydropyran-4-yl)ethoxy)naphthalen-1-yl]urea |
| 231 | 1-[2-carbomethoxy-5-tert-butyl-3-thienyl]-3-[4-(2-(1-oxo- |
| | tetrahydrothiophen-3-yl)ethoxy)naphthalen-1-yl]urea |
| 232 | 1-[2-methylcarbamoyl-5-tert-butyl-3-thienyl]-3-[4-(2-(1-oxo- |
| | tetrahydrothiophen-3-yl)ethoxy)naphthalen-1-yl]urea |
| 233 | 1-[2-carbomethoxy-5-tert-butyl-3-thienyl]-3-[4-(pyridin-4-yl- |
| | methoxy)naphthalen-1-yl]urea |
| 234 | 1-[2-methylcarbamoyl-5-tert-butyl-3-thienyl]-3-[4-(pyridin-4- |
| | yl)methoxy)naphthalen-1-yl]urea |
| 235 | 1-[2-carbomethoxy-5-tert-butyl-3-thienyl]-3-[4-(2-imidazol-1-yl- |
| | ethoxy)naphthalen-1-yl]urea |
| 236 | 1-[2-carbomethoxy-5-tert-butyl-3-thienyl]-3-[4-(2-(3,4- |
| | dimethoxyphenyl)-ethoxy)naphthalen-1-yl]urea |
| 237 | 1-[2-methylcarbamoyl-5-tert-butyl-3-thienyl]-3-[4-(2-(3,4- |
| | dimethoxyphenyl)ethoxy)naphthalen-1-yl]urea |
| 238 | 1-[2-carbomethoxy-5-tert-butyl-3-pyrrolyl]-3-[4-(2-morpholin-4-yl- |
| | ethoxy)naphthalen-1-yl]urea |
| 239 | 1-[2-methylcarbamoyl-5-tert-butyl-3-pyrrolyl]-3-[4- |
| | (2-morpholin-4-yl-ethoxy)naphthalen-1-yl]urea |
| 240 | 1-[1-methyl-2-carbomethoxy-5-tert-butyl-3-pyrrolyl]-3-[4-(2- |
| | morpholin-4-yl-ethoxy)naphthalen-1-yl]urea |
| 241 | 1-[2-carbomethoxy-5-tert-butyl-3-pyrrolyl]-3-[4-(2-(1- |
| | oxothiomorpholin-4-yl)ethoxy)naphthalen-1-yl]urea |
| 242 | 1-[2-methylcarbamoyl-5-tert-butyl-3-pyrrolyl]-3-[4-(2-(1- |
| | oxothiomorpholin-4-yl)ethoxy)naphthalen-1-yl]urea |
| 243 | 1-[1-methyl-2-carbomethoxy-5-tert-butyl-3-pyrrolyl]-3-[4-(2-(1- |
| | oxothiomorpholin-4-yl)ethoxy)naphthalen-1-yl]urea |
| 244 | 1-[2-carbomethoxy-5-tert-butyl-3-pyrrolyl]-3-[4-(2- |
| | (tetrahydropyran-4-yl)ethoxy)naphthalen-1-yl]urea |
| 245 | 1-[2-methylcarbamoyl-5-tert-butyl-3-pyrrolyl]-3-[4-(2- |
| | (tetrahydropyran-4-yl)ethoxy)naphthalen-1-yl]urea |
| 246 | 1-[2-carbomethoxy-5-tert-butyl-3-pyrrolyl]-3-[4-(2-(1-oxo- |
| | tetrahydrothiophen-3-yl)ethoxy)naphthalen-1-yl]urea |
| 247 | 1-[2-methylcarbamoyl-5-tert-butyl-3-pyrrolyl]-3-[4-(2-(1-oxo- |
| | tetrahydrothiophen-3-yl)ethoxy)naphthalen-1-yl]urea |
| 248 | 1-[1-methyl-2-carbomethoxy-5-tert-butyl-3-pyrrolyl]-3-[4-(2-(1- |
| | tetrahydrothiophen-3-yl)ethoxy)naphthalen-1-yl]urea |
| 249 | 1-[2-carbomethoxy-5-tert-butyl-3-pyrrolyl]-3-[4-(pyridin-4-yl- |
| | methoxy )naphthalen-1-yl]urea |
| 250 | 1-[2-methylcarbamoyl-5-tert-butyl-3-pyrrolyl]-3-[4-(pyridin-4-yl- |
| | methoxy)naphthalen-1-yl]urea |
| 251 | 1-[2-carbomethoxy-5-tert-butyl-3-pyrrolyl]-3-[4-(pyridin-4-yl- |
| | ethoxy)naphthalen-1-yl]urea |
| 252 | 1-[2-carbomethoxy-5-tert-butyl-3-pyrrolyl]-3-[4-(2-imidazol-1-yl- |
| | ethoxy)naphthalen-1-yl]urea |
| 253 | 1-[2-carbomethoxy-5-tert-butyl-3-pyrrolyl]-3-[4-(2-(3,4- |
| | dimethoxyphenyl)ethoxy)naphthalen-1-yl]urea |
| 254 | 1-[2-methylcarbamoyl-5-tert-butyl-3-pyrrolyl]-3-[4-(2-(3,4- |
| | dimethoxyphenyl)ethoxy)naphthalen-1-yl]urea |

TABLE 5
WO 01/36403

| Ex- | |
|---|---|
| am- | |
| ple | |
| Num- | |
| ber | Compound Name |
| 255 | 1-(5-tert-butyl-2-methoxyphenyl)-3-[4-(2-methoxy-pyridin-4- |
| | yl-oxy)-naphthalen-1-yl]urea |
| 256 | 1-(5-tert-butyl-2-methoxyphenyl)-3-[4-(2-methyl-pyridin-4-yl-oxy)- |
| | naphthalen-1-yl]urea |
| 257 | 1-(5-tert-butyl-2,3-dimethoxyphenyl)-3-[4-(2-methoxy-pyridin- |
| | 4-yl-oxy)-naphthalen-1-yl]urea |
| 258 | 5-tert-butyl-2-methoxy-3-{3-(pyridin-4-yl-oxy)naphthalen-1- |
| | yl]ureido}benzamide |
| 259 | Morpholine carboxylic acid (5-tert-butyl-2-methoxy-3-{3-[- |
| | (pyridin-4-yloxy)naphthalen-1-yl}ureido}phenyl)amide |
| 260 | N-(5-tert-butyl-2-methoxy-3-{3-[-(pyridin4-yl-oxy)naphthalen-1- |
| | yl]ureido}phenyl)acetamide |
| 261 | 3-(5-tert-butyl-2-methoxy-3-{3-[-(pyridin-4-yl-oxy)naphthalen-1- |
| | yl]ureido}phenyl)-1,1-dimethylurea |
| 262 | 1-(5-tert-butyl-2,3-dimethoxy-phenyl)-3-[4-(2-amino-pyridin-4- |
| | yloxy)-naphthalen-1-yl]urea |
| 263 | 1-(5-tert-butyl-2,3-dimethoxyphenyl)-3-[4-(2-methylamino- |
| | pyridin-4-yloxy]-naphthalen-1-yl}urea |
| 264 | 1-(5-tert-butyl-2,3-dimethoxyphenyl)-3-{4-[2-(1-phenyl- |
| | ethylamino)-pyridin-4-yloxy]-naphthalen-1-yl}urea |
| 265 | 1-(5-tert-butyl-2-methoxy-pyridin-3-yl)-3-[4-(2-amino-pyridin-4- |
| | yloxy)-naphthalen-1-yl]urea |
| 266 | 1-(5-tert-butyl-2-methoxy-pyridin-3-yl)-3-[4-(2-methylamino- |
| | pyridin-4-yloxy]-naphthalen-1-yl}urea |
| 267 | 1-(5-tert-butyl-2-methoxy-pyridin-3-yl)-3-{4-[2-(1-phenyl- |
| | ethylamino)-pyridin-4-yloxy]-naphthalen-1-yl}urea |
| 268 | 1-(5-tert-butyl-2-methyl-phenyl)-3-[4-(2-aminopyridin-4-yl-oxy)- |
| | naphthalen-1-yl]urea |
| 269 | 1-(5-tert-butyl-2-morpholin-4-yl-phenyl)-3-[4-(2-morpholin-4-yl- |
| | ethoxy)-naphthalen-1-yl]urea |
| 270 | 1-(5-tert-butyl-2-methoxyphenyl)-3-[4-(2-methylpyridin-4-yl-oxy)- |
| | naphthalen-1-yl]urea |
| 271 | 1-(5-tert-butyl-2,3-dimethoxyphenyl)-3-[4-(2-methoxypyridin-4-yl- |
| | oxy)-naphthalen-1-yl]urea |
| 272 | 5-tert-butyl-2-methoxy-3-{3-[-(pyridin-4-yl-oxy)naphthalen-1- |
| | yl]ureido}benzamide |
| 273 | Morpholine-4-carboxylic acid (5-tert-butyl-2-methoxy-3-{3-[- |
| | (pyridin-4-yl-oxy)naphthalen-1-yl]ureido}phenyl)amide |
| 274 | N-(5-tert-butyl-2-methoxy-3-{3-[-(pyridin-4-yl-oxy)naphthalen-1- |
| | yl]}ureido}phenyl)acetamide |
| 275 | 3-(5-tert-butyl-2-methoxy-3-{3-[-(pyridin-4-yl-oxy)naphthalen-1- |
| | yl]ureido}phenyl)-1,1-dimethylurea |
| 276 | 1-(5-tert-butyl-2,3-dimethoxy-phenyl)-3-[4-(2-amino-pyridin-4- |
| | yloxy)-naphyhalen-1-yl]urea |
| 277 | 1-(5-tert-butyl-2,3-dimethoxy-phenyl)-3-[4-(2-methylamino- |
| | pyridin-4-yloxy)-naphthalen-1-yl]urea |
| 278 | 1-(5-tert-butyl-2,3-dimethoxy-phenyl)-3-[4-(2-phenyl-ethylamino- |
| | pyridin-4-yloxy)-naphthalen-1-yl]urea |
| 279 | 1-(5-tert-butyl-2-methoxy-pyridin-3-yl)-3-[4-(2-amino-pyridin-4- |
| | yloxy)-naphthalen-1-yl]urea |
| 280 | 1-(5-tert-butyl-2-methoxy-pyridin-3-yl)-3-[4-(2-methylamino- |
| | pyridin-4-yloxy)-naphthalen-1-yl]urea |
| 281 | 1-(5-tert-butyl-2-methoxy-pyridin-3-yl)-3-{4-[2-(1-phenyl- |
| | ethylamino)-pyridin-4-yloxy)-naphthalen-1-yl]urea |
| 282 | 1-(5-tert-butyl-2-methyl-phenyl)-3-[4-(2-aminopyridin-4-yloxy)- |
| | naphthalen-1-yl]urea |
| 283 | 1-(5-tert-butyl-2-morpholin-4-yl-phenyl)-3-[4-(2-morpholin-4-yl- |
| | ethoxy)-naphthalen-1-yl]urea |

What is claimed is:

1. A method for treating or preventing a disease in a human or other mammal regulated by tyrosine kinase,(associated with an aberration in the tyrosine kinase signal transduction pathway) comprising administering to a human or other mammal in need thereof one or more compounds selected from the group consisting of:
a) ureas selected from the group consisting of:

| | |
|---|---|
| 1 | 1-[5-tert-butyl-2-(2-methylpyridin-5-yl)-2H-pyrazol-3-yl]-3-(4- |
| | chlorophenyl)urea |
| 2 | 1-(5-tert-butyl-2-p-tolyl-2H-pyrazol-3-yl)-3-(4-methoxynaphthalen-1- |
| | yl)urea |
| 3 | 1-[5-tert-butyl-2-(3,4-dimethylphenyl)-2H-pyrazol-3-yl]-3-(4- |
| | fluorophenyl)urea |
| 4 | 1-[5-tert-butyl-2-(pyridin-3-yl)-2H-pyrazol-3-yl]-3-(4- |
| | cyanonaphthalen-1-yl)urea |
| 5 | 1-[5-tert-butyl-2-p-tolyl-2H-pyrazol-3-yl]-3-[4-(2-morpholin-4- |
| | ylethoxy)naphthalen-1-yl]-urea |
| 6 | 1-[5-tert-butyl-2-p-tolyl-2H-pyrazol-3-yl]-3-[4-(2-(cis-2,6-dimethylmorpholin- |
| | 4-yl)ethoxy)naphthalene-1-yl]-urea |
| 7 | 1-[5-tert-butyl-2-p-tolyl-2H-pyrazol-3-yl]-3-[4-(2-(trans-2,6- |
| | dimethylmorpholin-4-yl)ethoxy)naphthalene-1-yl]-urea |
| 8 | 1-[5-tert-butyl-2-p-tolyl-2H-pyrazol-3-yl]-3-[4-(2-(2- |
| | (methoxymethyl)morpholin-4-yl)ethoxy)naphthalen-1-yl]-urea |
| 9 | 1-[5-tert-butyl-2-p-tolyl-2H-pyrazol-3-yl]-3-[4-(2-(morpholin-4-yl)-2- |
| | oxoethoxy)naphthalen-1-yl]-urea |
| 10 | 1-[5-tert-butyl-2-p-tolyl-2H-pyrazol-3-yl]-3-[4-(2-(morpholin-4-yl)-2- |
| | methylethoxy)naphthalen-1-yl]-urea |
| 11 | 1-[5-tert-butyl-2-p-tolyl-2H-pyrazol-3-yl]-3-[4-(2-(morpholin-4-yl)-1- |
| | methylethoxy)naphthalen-1-yl]-urea |
| 12 | 1-[5-tert-butyl-2-p-tolyl-2H-pyrazol-3-yl]-3-[4-(2-thiomorpholin-4- |
| | ylethoxy)naphthalen-1-yl]-urea |
| 13 | 1-[5-tert-butyl-2-p-tolyl-2H-pyrazol-3-yl]-3-[4-(2-(1-oxothiomorpholin-4-yl) |
| | ethoxy)naphthalen-1-yl]-urea |
| 14 | 1-[5-tert-butyl-2-p-tolyl-2H-pyrazol-3-yl]-3-[4-(2-morpholin-4-yl- |
| | ethoxy)-3-methylnaphthalen-1-yl]-urea |
| 15 | 1-[5-tert-butyl-2-p-tolyl-2H-pyrazol-3-yl]-3-[4-(2-(morpholin-4-yl- |
| | carbonyloxo)ethoxy)naphthalen-1-yl]-urea |
| 16 | 1-[5-tert-butyl-2-p-tolyl-2H-pyrazol-3-yl]-3-[4-(2-(tetrahydropyran-4- |
| | yl)ethoxy)naphthalen-1-yl]-urea |
| 17 | 1-[5-tert-butyl-2-p-tolyl-2H-pyrazol-3-yl]-3-[4-(2-(1-oxo- |
| | tetrahydrothiophen-3-yl)ethoxy)naphthalen-1-yl]-urea |
| 18 | 1-[5-tert-butyl-2-p-tolyl-2H-pyrazol-3-yl]-3-[4-(3-morpholin-4-yl- |
| | propyl)naphthalen-1-yl]-urea |
| 19 | 1-[5-tert-butyl-2-p-tolyl-2H-pyrazol-3-yl]-3-[4-(3-morpholin-4-yl- |
| | methyl)naphthalen-1-yl]-urea |
| 20 | 1-[5-tert-butyl-2-p-tolyl-2H-pyrazol-3-yl]-3-[4-(2-pyridin-4-yl- |
| | ethyl)naphthalen-1-yl]-urea |
| 21 | 1-[5-tert-butyl-2-p-tolyl-2H-pyrazol-3-yl]-3-[4-(3-(morpholin-4- |
| | yl)propyn-1-yl)naphthalen-1-yl]-urea |
| 22 | 1-[5-tert-butyl-2-p-tolyl-2H-pyrazol-3-yl]-3-[4-(3-(tetrahydropyran-2- |
| | yl-oxy)propyn-1-yl)naphthalen-1-yl]-urea |
| 23 | 1-[5-tert-butyl-2-p-tolyl-2H-pyrazol-3-yl]-3-[4-(3-(tetrahydropyran-2- |
| | yl-oxy)butyn-1-yl)naphthalen-1-yl]-urea |
| 24 | 1-[5-tert-butyl-2-p-tolyl-2H-pyrazol-3-yl]-3-[4-(3-(piperidin-1- |
| | yl)propyn-1-yl)naphthalen-1-yl]-urea |
| 25 | 1-[5-tert-butyl-2-p-tolyl-2H-pyrazol-3-yl]-3-[4-(3-(2- |
| | methoxymethylmorpholin-4-yl)propyn-1-yl)naphthalen-1-yl]-urea |
| 26 | 1-[5-tert-butyl-2-p-tolyl-2H-pyrazol-3-yl]-3-[4-(pyridin-4-yl- |
| | methoxy)naphthalen-1-yl]-urea |
| 27 | 1-[5-tert-butyl-2-p-tolyl-2H-pyrazol-3-yl]-3-[4-(2-pyridin-4-yl- |
| | ethoxy)naphthalen-1-yl]-urea |
| 28 | 1-[5-tert-butyl-2-p-tolyl-2H-pyrazol-3-yl]-3-[4-(3-pyridin-4-yl- |
| | propoxy)naphthalen-1-yl]-urea |
| 29 | 1-[5-tert-butyl-2-p-tolyl-2H-pyrazol-3-yl]-3-[4-(2-imidazol-1-yl- |
| | ethoxy)naphthalen-1-yl]-urea |
| 30 | 1-[5-tert-butyl-2-p-tolyl-2H-pyrazol-3-yl]-3-[4-(2-(3,4- |
| | dimethoxyphenyl)-ethoxy)naphthalen-1-yl]-urea |
| 31 | 1-[5-tert-butyl-2-p-tolyl-2H-pyrazol-3-yl]-3-[4-(pyridin-4-yl- |
| | methylamino)naphthalen-1-yl]-urea |
| 32 | 1-[5-isopropyl-2-phenyl-2H-pyrazol-3-yl]-3-[4-(2-morpholin-4-yl-ethoxy) |
| | naphthalen-1-yl]-urea |
| 33 | 1-[5-cyclohexyl-2-phenyl-2H-pyrazol-3-yl]-3-[4-(2-morpholin-4-yl- |
| | ethoxy)naphthalen-1-yl]-urea |
| 34 | 1-[5-(2,2,2-trifluoroethyl)-2-phenyl-2H-pyrazol-3-yl]-3-[4-(2-morpholin-4-yl- |
| | ethoxy)naphthalen-1-yl]-urea |
| 35 | 1-[5-(1-methylcycloprop-1-yl)-2-phenyl-2H-pyrazol-3-yl]-3-[4-(2- |
| | morpholin-4-yl-ethoxy)naphthalen-1-yl]-urea |
| 36 | 1-[5-(1-methylcyclohex-1-yl)-2-phenyl-2H-pyrazol-3-yl]-3-[4-(2-morpholin-4- |
| | yl-ethoxy)naphthalen-1-yl]-urea |
| 37 | 1-[5-tert-butyl-2-methyl-2H-pyrazol-3-yl]-3-[4-(2-morpholin-4-yl- |
| | ethoxy)naphthalen-1-yl]-urea |
| 38 | 1-[5-tert-butyl-2-(4-chlorophenyl)-2H-pyrazol-3-yl]-3-[4-(2- |
| | morpholin-4-yl-ethoxy)naphthalen-1-yl]-urea |
| 39 | 1-[5-tert-butyl-2-butyl-2H-pyrazol-3-yl]-3-[4-(2-morpholin-4-yl- |
| | ethoxy)naphthalen-1-yl]-urea |
| 40 | 1-[5-tert-butyl-2-(4-methyl-3-carbamylphenyl)-2H-pyrazol-3-yl]-3- |
| | [4-(2-morpholin-4-yl-ethoxy)naphthalen-1-yl]-urea |
| 41 | 1-[5-tert-butyl-2-(4-methyl-3-(morpholin-4-yl)methylphenyl)-2H- |
| | pyrazol-3-yl]-3-[4-(2-morpholin-4-yl-ethoxy)naphthalen-1-yl ]-urea |
| 42 | 1-[5-tert-butyl-2-(4-methyl-3-dimethylaminomethylphenyl)-2H-pyrazol-3-yl]-3- |
| | [4-(2-morpholin-4-yl-ethoxy)naphthalen-1-yl]-urea |
| 43 | 1-[5-tert-butyl-2-(3-dimethylaminomethylphenyl)-2H-pyrazol-3-yl]- |
| | 3-[4-(2-morpholin-4-yl-ethoxy)naphthalen-1-yl]-urea |
| 44 | 1-[5-tert-butyl-2-(2-chloropyridin-5-yl)-2H-pyrazol-3-yl]-3-[4-(2- |
| | morpholin-4-yl-ethoxy)naphthalen-1-yl]-urea |
| 45 | 1-[5-tert-butyl-2-(2-methylpyridin-5-yl)-2H-pyrazol-3-yl]-3-[4-(2- |
| | morpholin-4-yl-ethoxy)naphthalen-1-yl]-urea |
| 46 | 1-[5-tert-butyl-2-(2-methoxypyridin-5-yl)-2H-pyrazol-3-yl]-3-[4-(2- |
| | morpholin-4-yl-ethoxy)naphthalen-1-yl]-urea |
| 47 | 1-[5-tert-butyl-2-(pyridin-3-yl)-2H-pyrazol-3-yl]-3-[4-(2-morpholin- |
| | 4-yl-ethoxy)naphthalen-1-yl]-urea |
| 48 | 1-[5-tert-butyl-2-(2-methylpyridin-5-yl)-2H-pyrazol-3-yl]-3-[4-(2- |
| | pyridin-4-yl-ethoxy)naphthalen-1-yl]-urea |
| 49 | 1-[5-tert-butyl-2-(2-methylpyridin-5-yl)-2H-pyrazol-3-yl]-3-[4-(2-(trans-2,6- |
| | dimethylmorpholin-4-yl)ethoxy)naphthalen-1-yl]-urea |
| 50 | 1-[5-tert-butyl-2-(2-methylpyridin-5-yl)-2H-pyrazol-3-yl]-3-[4-(3- |
| | morpholin-4-yl-propyn-1-yl)naphthalen-1-yl]-urea |
| 51 | 1-[5-(2-hydroxy-1,1-dimethyl-ethyl)-2-p-tolyl-2H-pyrazol-3-yl]-3-[4- |
| | (2-morpholin-4-yl-ethoxy)naphthalen-1-yl]-urea |
| 52 | 1-[5-tert-butyl-2-(3-hydroxy-4-methyl-phenyl)-2H-pyrazol-3-yl]-3-[4- |
| | (2-morpholin-4-yl-ethoxy)naphthalen-1-yl]-urea |
| 53 | 1-[5-tert-butyl-2-(4-hydroxymethyl-phenyl)-2H-pyrazol-3-yl]-3-[4-(2-morpholin- |
| | 4-yl-ethoxy)naphthalen-1-yl]-urea |
| 54 | 1-[5-tert-butyl-2-p-tolyl-2H-pyrazol-3-yl]-3-{4-[2-(3-oxo-morpholin- |
| | 4-yl)-ethoxy]naphthalen-1-yl}-urea |
| 55 | 1-[5-tert-butyl-2-p-tolyl-2H-pyrazol-3-yl]-3-{4-[2-(4-hydroxy- |
| | morpholin-4-yl)-ethoxy]naphthalen-1-yl}-urea |
| 56 | 1-[5-(2-hydroxy-1,1-dimethyl-ethyl)-2-(6-methyl-pyridin-3-yl)-2H- |
| | pyrazol-3-yl]-3-[4-(2-morpholin-4-yl-ethoxy)naphthalen-1-yl]-urea |
| 57 | 1-[5-tert-butyl-2-(1-hydroxy-6-methyl-pyridin-3-yl)-2H-pyrazol-3- |
| | yl]-3-[4-(2-morpholin-4-yl-ethoxy)naphthalene-1-yl]-urea |
| 58 | 1-[5-tert-buytl-2-(6-methyl-pyridin-3-yl)-2H-pyrazol-3-yl]-3-{4-[2- |
| | (4-hydroxy-morpholin-4-yl)-ethoxy]naphthalen-1-yl}-urea |
| 59 | 1-[5-(2-hydroxy-1,1-dimethyl-ethyl)-2-(6-methyl-pyridin-3-yl)-2H- |
| | pyrazol-3-yl]-3-[4-(2-pyridin-4-yl-ethoxy)naphthalen-1-yl]-urea |
| 60 | 1-[5-tert-butyl-2-(6-methyl-pyridin-3-yl)-2H-pyrazol-3-yl]-3-[4-(2- |
| | hydroxy-2-pyridin-4-yl-ethoxy)naphthalen-1-yl]-urea |
| 61 | 1-[5-tert-butyl-2-(6-methyl-pyridin-3-yl)-2H-pyrazol-3-yl]-3-{4-[2- |
| | (1-hydroxy-2-pyridin-4-yl)-ethoxy]naphthalene-1-yl}-urea |
| 62 | 1-[5-(2-hydroxy-1,1-dimethyl-ethyl)-2-p-tolyl-2H-pyrazol-3-yl]-3-{4- |
| | [2-(1-oxo-thiomorpholin-4-yl)-ethoxy]naphthalen-1-yl}-urea |
| 63 | 1-[5-tert-butyl-2-(4-hydroxymethyl-phenyl)-2H-pyrazol-3-yl]-3-{4- |
| | [2-(1-oxo-thiomorpholin-4-yl)-ethoxy]naphthalen-1-yl}-urea |
| 64 | 1-[5-tert-butyl-2-p-tolyl-2H-pyrazol-3-yl]-3-{4-[2-(1,3-dioxo- |
| | thiomorpholin-4-yl)-ethoxy]naphthalen-1-yl}-urea |
| 65 | 1-[5-(2-hydroxy-1,1-dimethyl-ethyl)-2-methyl-2H-pyrazol-3-yl]-3-[4- |
| | (2-morpholin-4-yl-ethoxy)-naphthalen-1-yl]-urea |
| 66 | 1-[5-tert-butyl-2-methyl-2H-pyrazol-3-yl]-3-{4-[2-(4-hydroxy- |
| | morpholin-4-yl)-ethoxy]-naphthalen-1-yl}-urea |
| 67 | 1-[5-tert-butyl-2-p-tolyl-2H-pyrazolyl-3-yl]-3-[4-(4-(morpholin-4-yl-methyl) |
| | phenyl)naphthalen-1-yl]urea |
| 68 | 1-[5-tert-butyl-2-p-tolyl-2H-pyrazolyl-3-yl]-3-[4-(4-(2-(morpholin-4- |
| | yl)ethyl)phenyl)naphthalen-1-yl]urea |
| 69 | 1-[5-tert-butyl-2-p-tolyl-2H-pyrazolyl-3-yl]-3-[4-(3-(morpholin-4-yl- |
| | methyl)phenyl)naphthalen-1-yl]urea |
| 70 | 1-[5-tert-butyl-2-p-tolyl-2H-pyrazolyl-3-yl]-3-[4-(6-(morpholin-4- |
| | ylmethyl-pyridin-3-yl)naphthalen-1-yl]urea |
| 71 | 1-[5-tert-butyl-2-p-tolyl-2H-pyrazolyl-3-yl]-3-[4-(5-(morpholin-4- |
| | ylmethyl-pyridin-2-yl)naphthalen-1-yl]urea |
| 72 | 1-[5-tert-butyl-2-p-tolyl-2H-pyrazolyl-3-yl]-3-[4-(5-(morpholin-4- |
| | ylmethyl-fur-2-yl)naphthalen-1-yl]urea |
| 73 | 1-[5-tert-butyl-2-(6-methyl-pyridin-3-yl)-2H-pyrazolyl-3-yl]-3-[4-(6- |
| | (morpholin-4-ylmethyl-pyridin-3-yl)naphthalen-1-yl]urea |
| 74 | 1-[5-tert-butyl-2-methyl-2H-pyrazolyl-3-yl]-3-[4-(6-morpholin-4- |
| | ylmethyl-pyridin-3-yl)naphthalen-1-yl]urea |
| 75 | 1-[5-tert-butyl-2-p-tolyl-2H-pyrazolyl-3-yl]-3-[4-(5-(morpholin-4-yl- |
| | methyl)pyridin-2-yl)naphthalen-1-yl]urea |
| 76 | 1-[5-tert-butyl-2-p-tolyl-2H-pyrazolyl-3-yl]-3-[4-(6-(morpholin-4-yl- |
| | methyl)pyridin-3-yl)naphthalen-1-yl]urea |
| 77 | 1-[5-tert-butyl-2-(6-methyl-pyridin-3-yl)-2H-pyrazolyl-3-yl]-3-[4-(3- |
| | (2-(pyridin-2-yl)ethylamino)cyclohexenyl)-naphthalen-1-yl]urea |
| 78 | 1-[5-tert-butyl-2-p-tolyl-2H-pyrazolyl-3-yl]-3-[4-(4-(pyridin-3-yl- |
| | methylaminomethyl)phenyl)naphthalen-1-yl]urea |
| 79 | 1-[5-tert-butyl-2-(6-methyl-pyridin-3-yl)-2H-pyrazolyl-3-yl]-3-[4-(4- |
| | (morpholin-4-yl-methyl)phenyl)naphthalene-1-yl]urea |
| 80 | 1-[5-tert-butyl-2-(6-methyl-pyridin-3-yl)-2H-pyrazolyl-3-yl]-3-[4-(6- |
| | (4-(hydroxybutylamino)pyridin-3-yl)-naphthalen-1-yl]urea |
| 81 | 1-[5-tert-butyl-2-(4-methyl-3-carbamylphenyl)-2H-pyrazolyl-3-yl]-3- |
| | [4-(6-(morpholin-4-yl-methyl)pyridin-3-yl)naphthalen-1-yl]urea |
| 82 | 1-[5-tert-butyl-2-(6-methyl-pyridin-3-yl)-2H-pyrazolyl-3-yl]-3-[4-(4- |
| | (3-hydroxypiuperidin-1-yl-methyl)phenyl)naphthalen-1-yl]urea |
| 83 | 1-[5-tert-butyl-2-(6-methyl-pyridin-3-yl)-2H-pyrazolyl-3-yl]-3-[4-(4- |
| | (4-hydroxymorpholin-4-yl-methyl)phenyl)naphthalen-1-yl]urea |
| 84 | 1-[5-tert-butyl-2-(6-methyl-pyridin-3-yl)-2H-pyrazolyl-3-yl]-3-[4-(3- |
| | (morpholin-4-yl-methyl)cyclohexenyl)naphthalen-1-yl]urea |
| 85 | 1-[5-tert-butyl-2-(6-methyl-pyridin-3-yl)-2H-pyrazolyl-3-yl]-3-[4-(4- |
| | (tetrahydrofuran-3-yl-methyl)-3-hydroxyphenyl)naphthalen-1-yl]urea |
| 86 | 1-[5-tert-butyl-2-(6-methyl-pyridin-3-yl)-2H-pyrazolyl-3-yl]-3-[4-(4- |
| | (N,N-di-(2-methoxyethyl)aminomethyl)phenyl)naphthalen-1-yl]urea |
| 87 | 1-[5-tert-butyl-2-(6-methyl-pyridin-3-yl)-2H-pyrazolyl-3-yl]-3-[4-(6- |
| | (cyanopropoxy)pyridin-3-yl)naphthalen-1-yl]urea |
| 88 | 1-[5-tert-butyl-2-(6-methyl-pyridin-3-yl)-2H-pyrazolyl-3-yl]-3-[4-(4- |
| | morpholin-4-yl-methyl-piperidinyl)naphthalen-1-yl]urea |
| 89 | 1-[5-tert-butyl-2-(6-methyl-pyridin-3-yl)-2H-pyrazolyl-3-yl]-3-[4-(4- |
| | (N,N-di-(2-cyanoethyl)aminomethyl)phenyl)naphthalen-1-yl]urea |
| 90 | 1-[5-tert-butyl-2-(6-methyl-pyridin-3-yl)-2H-pyrazolyl-3-yl]-3-[4-(4- |
| | (furan-2-yl-methyl)-3-hydroxyphenyl)naphthalen-1-yl]urea |
| 91 | 1-[5-tert-butyl-2-(6-methyl-pyridin-3-yl)-2H-pyrazolyl-3-yl]-3-[4-(4- |
| | (thiomorpholin-4-yl-methyl)phenyl)naphthalen-1-yl]urea |
| 92 | 1-[5-tert-butyl-2-(6-methyl-pyridin-3-yl)-2H-pyrazolyl-3-yl]-3-[4-(4- |
| | (3-carboxamidopiperidin-1-yl-methyl)phenyl)naphthalen-1-yl]urea |
| 93 | 1-[5-tert-butyl-2-(6-methyl-pyridin-3-yl)-2H-pyrazolyl-3-yl]-3-[4-(4- |
| | (2-methyl-3-oxo-piperzin-1-yl-methyl)phenyl)naphthalen-1-yl]urea |
| 94 | 1-[5-tert-butyl-2-(2-methylpyrimidin-5-yl)-2H-pyrazolyl-3-yl]-3-[4- |
| | (6-(morpholin-4-yl-methyl)pyridin-3-yl)naphthalen-1-yl]urea |
| 95 | 1-[5-tert-butyl-2-(6-methyl-pyridin-3-yl)-2H-pyrazolyl-3-yl]-3-[4-(6- |
| | (4-hydroxybutyloxy)pyridin-3-yl)naphthalen-1-yl]urea |
| 96 | 1-[3-tert-butyl-1′H-[1,4′]bipyrazol-5-yl]-3-[4-(6-(morpholin-4-yl-methyl)pyridin- |
| | 3-yl)naphthalen-1-yl]urea |
| 97 | 1-[5-tert-butyl-2-2H-pyrazolyl-3-yl]-3-[4-(6-(tetrahydrothiopyran-4- |
| | yl-amino)pyridin-3-yl)naphthalen-1-yl]urea |
| 98 | 1-[5-tert-butyl-2-(2-cyanoethyl)-2H-pyrazolyl-3-yl]-3-[4-(6- |
| | (morpholin-4-yl-methyl)pyridin-3-yl)naphthalen-1-yl]urea |
| 99 | 1-[5-tert-butyl-2-(6-methyl-pyridin-3-yl)-2H-pyrazolyl-3-yl]-3-[4-(6- |
| | (2,6-dim ethylmorpholin-4-yl-methyl)pyridin-3-yl)naphthalen-1- |
| | yl]urea |
| 100 | 1-[5-tert-butyl-2-(2-methoxypyridin-5-yl)-2H-pyrazolyl-3-yl]-3-[4-(6- |
| | (morpholin-4-yl-methyl)pyridin-3-yl)naphthalen-1-yl]urea |
| 101 | 1-[5-tert-butyl-2-(2-aminopyridin-5-yl)-2H-pyrazolyl-3-yl]-3-[4-(6- |
| | (morpholin-4-yl-methyl)pyridin-3-yl)naphthalen-1-yl]urea |
| 102 | 1-[5-tert-butyl-2-(6-methyl-pyridin-3-yl)-2H-pyrazolyl-3-yl]-3-[4-(6- |
| | (morpholin-4-yl-4-carbonyl)pyridin-3-yl)naphthalen-1-yl]urea |
| 103 | 1-[5-tert-butyl-2-(6-methyl-pyridin-3-yl)-2H-pyrazolyl-3-yl]-3-[4-(6- |
| | (2-oxa-5-aza-bicyclo[2.2.1]hept-5-yl-methyl)pyridin-3-yl)naphthalen- |
| | 1-yl]urea |
| 104 | 1-[5-tert-butyl-2-(6-methyl-pyridin-3-yl)-2H-pyrazolyl-3-yl]-3-[4-(4- |
| | (N-(2-cyanoethyl)-N-(pyridin-3- |
| | ylmethyl)aminomethyl)phenyl)naphthalene-1-yl]urea |
| 105 | 1-[5-tert-butyl-2-(6-methyl-pyridin-3-yl)-2H-pyrazolyl-3-yl]-3-[4-(4- |
| | (N-(2-cyanoethyl)-N-(tetrahydrofuran-2-yl- |
| | methyl)aminomethyl)phenyl)naphthalen-1-yl]urea |
| 106 | 1-[5-tert-butyl-2-(6-methyl-pyridin-3-yl)-2H-pyrazolyl-3-yl]-3-[4-(6- |
| | (morpholin-4-yl-methyl)-4-methoxypyridin-3-yl)naphthalen-1-yl]urea |
| 107 | 1-[5-tert-butyl-2-(6-methyl-pyridin-3-yl)-2H-pyrazolyl-3-yl]-3-[4-(6- |
| | (1-morpholin-4-yl-propyl)pyridin-3-yl)naphthalen-1-yl]urea |
| 108 | 1-[3-tert-butyl-1′-methyl-1′H-[1,4′]bipyrazol-5-yl]-3-[4-(6- |
| | (morpholin-4-yl-methyl)pyridin-3-yl)naphthalen-1-yl]urea |
| 109 | 1-[5-tert-butyl-2-(6-methyl-pyridin-3-yl)-2H-pyrazolyl-3-yl]-3-[4-(6- |
| | (1-oxo-tetrahydrothiopyran-4-yl-amino)pyridin-3-yl)naphthalen-1- |
| | yl]urea |
| 110 | 1-[5-tert-butyl-2-(6-methyl-pyridin-3-yl)-2H-pyrazolyl-3-yl]-3-[4-(6- |
| | (tetrahydropyran-4-yl-amino)pyridin-3-yl)naphthalen-1-yl]urea |
| 111 | 1-[5-tert-butyl-2-(6-methyl-pyridin-3-yl)-2H-pyrazolyl-3-yl]-3-[4-(5- |
| | (tetrahydrothiopyran-4-yl-aminbo)pyrazin-2-yl)naphthalen-1-yl]urea |
| 112 | 1-[5-tert-butyl-2-(6-methyl-pyridin-3-yl)-2H-pyrazolyl-3-yl]-3-[4-(6- |
| | (metrhylcarbonylamino)pyridin-3-yl)naphthalen-1-yl]urea |
| 113 | 1-[5-tert-butyl-1′-(3-methylsulfanylpropyl)-1′H-[1,4′]bipyrazol-5- |
| | yl]-3-[4-(6-(morpholin-4-yl-methyl)pyridin-3-yl)naphthalen-1-yl]urea |
| 114 | 1-[5-tert-butyl-2-(2-methylpyrimidin-5-yl)-2H-pyrazolyl-3-yl]-3-[4- |
| | (6-(1-oxo-thiomorpholin-4-yl-methyl)pyridin-3-yl)naphthalen-1- |
| | yl]urea |
| 115 | 1-[5-tert-butyl-2-(2-methylpyrimidin-5-yl)-2H-pyrazolyl-3-yl]-3-[4- |
| | (6-(tetrahydropyran-4-ylamino)pyridin-3-yl)naphthalen-1-yl]urea |
| 116 | 1-[5-tert-butyl-2-(2-methylthiopyrimidin-5-yl)-2H-pyrazolyl-3-yl]-3- |
| | [4-(6-(morpholin-4-yl-methyl)pyridin-3-yl)naphthalen-1-yl]urea |
| 117 | 1-[5-tert-butyl-2-(2-aminopyrimidin-5-yl)-2H-pyrazolyl-3-yl]-3-[4- |
| | (6-(morpholin-4-yl-methyl)pyridin-3-yl)naphthalen-1-yl]urea |
| 118 | 1-[3-tert-butyl-1′-methyl-1′H-[1,4;]-bipyrazol-5-yl]-3-[4-(6- |
| | (morpholin-4-yl-methyl)phenyl)naphthalen-1-yl]urea |
| 119 | 1-[5-tert-butyl-2-p-tolyl-2H-pyrazolyl-3-yl]-3-[4-(6-(1-oxo- |
| | tetrahydrothiopyran-4-yl-amino)pyridin-3-yl)naphthalen-1-yl]urea |
| 120 | 1-[5-tert-butyl-2-p-tolyl-2H-pyrazolyl-3-yl]-3-[4-(6-(thiomorpholin-4-yl-methyl) |
| | pyridin-3-yl)naphthalen-1-yl]urea |
| 121 | 1-[5-tert-butyl-2-p-tolyl-2H-pyrazolyl-3-yl]-3-[4-(2-(morpholin-4-yl- |
| | carbonyl)pyrimidin-5-yl)naphthalen-1-yl]urea |
| 122 | 1-[5-tert-butyl-2-p-tolyl-2H-pyrazolyl-3-yl]-3-[4-(4-(morpholin-4-yl- |
| | methyl)pyrimidin-5-yl)naphthalen-1-yl]urea |
| 123 | 1-[5-tert-butyl-2-p-tolyl-2H-pyrazolyl-3-yl]-3-[4-(6-(1-oxo- |
| | thiomorpholin-4-yl-methyl)pyridin-3-yl)naphthalen-1-yl]urea |
| 124 | 1-[5-tert-butyl-2-(2-methylpyrimidin-5-yl)-2H-pyrazolyl-3-yl]-3-[4- |
| | (2-(morpholin-4-yl-methyl)pyrimidin-5-yl)naphthalen-1-yl]urea |
| 125 | 1-[2-tert-butyl-5-methyl-pyridin-4-yl]-3-[4-(6-(morpholin-4-yl- |
| | methyl-pyridin-3-yl)naphthalen-1-yl]urea |
| 126 | 1-[3-tert-butyl-phenyl]-3-[4-(6-(morpholin-4-yl-methyl)pyridin-3- |
| | yl)naphthalen-1-yl]urea |
| 127 | 1-[4-methyl-biphenyl-3-yl]-3-[4-(6-(morpholin-4-yl-methyl-pyridin- |
| | 3-yl)naphthalen-1-yl]urea |
| 128 | 1-[4-tert-butyl-biphenyl-2-yl]-3-[4-(6-morpholin-4-yl-methyl-pyridin- |
| | 3-yl)naphthalen-1-yl]urea |
| 129 | 1-[5-isopropyl-2-methyl-phenyl]-3-[4-(6-morpholin-4-yl-methyl- |
| | pyridin-3-yl)naphthalen-1-yl]urea |
| 130 | 1-[5-sec-butyl-2-methoxy-phenyl]-3-[4-(6-(morpholin-4-yl-methyl- |
| | pyridin-3-yl)naphthalen-1-yl]urea |
| 131 | 1-[5-tert-butyl-2-methoxymethyl-phenyl]-3-[4-(6-(morpholin-4-yl- |
| | methyl-pyridin-3-yl)naphthalen-1-yl]urea |
| 132 | 1-[5-tert-butyl-2-methoxy-phenyl]-3-[4-(6-(morpholin-4-yl-methyl- |
| | pyridin-3-yl)naphthalen-1-yl]urea |
| 133 | 1-[5-tert-butyl-2-mewthyl-phenyl]-3-{6-[(3-(methoxy-propyl)- |
| | methyl-amino]-pyridin-3-yl}naphthalen-1-yl]urea |
| 134 | 1-[5-tert-butyl-2-methyl-phenyl]-3-[4-(6-morpholin-4-yl-methyl- |
| | pyridin-3-yl)naphthalen-1-yl]urea |
| 135 | 1-[5-tert-butyl-2-methyl-pyridin-3-yl]-3-[4-(6-morpholin-4-yl- |
| | methyl-pyridin-3-yl)naphthalen-1-yl]urea |
| 136 | 1-[5-(1,1-dimethylpropyl)-2-methoxy-phenyl]-3-[4-(6-morpholin-4- |
| | yl-methyl-pyridin-3-yl)naphthalen-1-yl]urea |
| 137 | 1-[5-tert-butyl-2-(1H-pyrazol-4-yl)-phenyl]-3-[4-(6-morpholin-4-yl-methyl- |
| | pyridin-3-yl)naphthalen-1-yl]urea |
| 138 | 1-[5-tert-butyl-2-(2-methyl-pyrimidin-5-yl)-phenyl]-3-[4-(6- |
| | morpholin-4-yl-methyl-pyridin-3-yl)naphthalen-1-yl]urea |
| 139 | 1-[5-tert-butyl-2-(3-hydroxy-propyl)-phenyl]-3-[4-(6-morpholin-4-yl-methyl- |
| | pyridin-3-yl)naphthalen-1-yl]urea |
| 140 | 1-[5-tert-butyl-2-(morpholine-4-carbonyl)-phenyl]-3-[4-(6- |
| | morpholin-4-yl-methyl-pyridin-3-yl)naphthalen-1-yl]urea |
| 141 | 1-[5-tert-butyl-2-methoxy-3-{3-[4-(6-morpholin-4-ylmethyl-pyridin- |
| | 3-yl)-naphthalen-1-yl]-ureido}-phenyl)-acetamide |
| 142 | 1-[3-methyl-naphthalen-2-yl]-3-[4-(6-morpholin-4-yl-methyl-pyridin- |
| | 3-yl)naphthalen-1-yl]urea |
| 143 | 1-[5-tert-butyl-2-methoxy-3-{3-[4-(6-morpholin-4-ylmethyl-pyridin- |
| | 3-yl)-naphthalen-1-yl]-urido}-phenyl)-acetamide |
| 144 | 1-[5-tert-butyl-3-(2,3-dihydroxy-propyl)-2-hydroxy-phenyl]-3-[4-(6- |
| | morpholin-4-yl-methyl-pyridin-3-yl)naphthalen-1-yl]urea |
| 145 | 1-[2,3-dimethyl-1H-indol-5-yl]-3-[4-(6-morpholin-4-yl-methyl- |
| | pyridin-3-yl)naphthalen-1-yl]urea |
| 146 | 1-{5-tert-butyl-2-methyl-3-[3-(tetrahydro-pyran-2-yloxy)-prop-1- |
| | ynyl]-phenyl}-3-[4-(6-morpholin-4-yl-methyl-pyridin-3- |
| | yl)naphthalen-1-yl]urea |
| 147 | 1-[2-methoxy-5-trifluoromethyl-phenyl]-3-[4-(6-morpholin-4-yl- |
| | methyl-pyrimidin-3-yl)naphthalene-1-yl]urea |
| 148 | 1-[5-(2,2-dimethyl-propionyl)-2-methyl-phenyl]-3-[4-(6-morpholin- |
| | 4-yl-methyl-pyridin-3-yl)naphthalen-1-yl]urea |
| 149 | 1-[5-tert-butyl-3-(3-hydroxy-prop-1-ynyl)-2-methyl-phenyl]-3-[4-(6- |
| | morpholin-4-yl-methyl-pyridin-3-yl)naphthalen-1-yl]urea |
| 150 | 1-[5-tert-butyl-2-(3-hydroxy-propyn-1-ynyl)-phenyl]-3-[4-(6- |
| | morpholin-4-yl-methyl-pyridin-3-yl)naphthalen-1-yl]urea |
| 151 | 1-[5-tert-butyl-3-(2,2-dimethyl-[1,3]dioxolan-4-ylmethyl)-2- |
| | methoxy-phenyl]-3-[4-(6-morpholin-4-yl-methyl-pyridin-3- |
| | yl)naphthalen-1-yl]urea |
| 152 | 1-[5-tert-butyl-3-(2,3-dihydroxy-propyl)-2-methoxy-phenyl]-3-[4-(6- |
| | morpholin-4-yl-methyl-pyridin-3-yl)naphthalen-1-yl]urea |
| 153 | 1-[5-tert-butyoxy-2-methoxy-phenyl]-3-[4-(6-morpholin-4-yl-methyl- |
| | pyridin-3-yl)naphthalen-1-yl]urea |
| 154 | 1-[5-(1-cyano-cyclopropyl)-2-methoxy-phenyl]-3-[4-(6-morpholin-4- |
| | yl-methyl-pyridin-3-yl)naphthalen-1-yl]urea |
| 155 | 1-[5-tert-butyl-3-(2-diethylamino-ethyl)-2-methoxy-phenyl]-3-[4-(6- |
| | morpholin-4-yl-methyl-pyridin-3-yl)naphthalen-1-yl]urea |
| 156 | 1-[5-tert-butyl-2-methoxy-phenyl]-3-[4-(6-[1,3]dioxolan-2-yl- |
| | pyridin-3-yl)naphthalen-1-yl]urea |
| 157 | 1-[5-tert-butyl-2-pyrrolidin-1-yl-phenyl]-3-[4-(6-morpholin-4-yl- |
| | methyl-pyridin-3-yl)naphthalen-1-yl]urea |
| 158 | 1-[5-tert-butyl-2-dimethylamino-phenyl]-3-[4-(6-morpholin-4-yl-methyl-pyridin- |
| | 3-yl)naphthalen-1-yl]urea |
| 159 | 1-[5-tert-butyl-2-propoxy-phenyl]-3-[4-(6-morpholin-4-yl-methyl- |
| | pyridin-3-yl)naphthalen-1-yl]urea |
| 160 | 1-[5-tert-butyl-2-methoxy-phenyl]-3-[4-(6-hydroxymethyl-pyridin-3- |
| | yl)naphthalen-1-yl]urea |
| 161 | 1-[5-tert-butyl-2-methoxy-phenyl]-3-{4-[6-(2,6-dimethyl-morpholin- |
| | 4-yl-methyl)pyridin-3-yl]naphthalen-1-yl}urea |
| 162 | 1-[5-cyclohexyl-2-methoxy-phenyl]-3-[4-(6-morpholin-4-yl-methyl- |
| | pyridin-3-yl)naphthalen-1-yl]urea |
| 163 | 1-[2,4-dimethoxy-5-trifluoromethyl-phenyl]-3-[4-(6-morpholin-4-yl- |
| | methyl-pyridin-3-yl)naphthalen-1-yl]urea |
| 164 | 1-[5-tert-butyl-2-methoxy-3-nitro-phenyl]-3-[4-(6-morpholin-4-yl- |
| | methyl-pyridin-3-yl)naphthalen-1-yl]urea |
| 165 | 1-[3-amino-5-tert-butyl-2-methoxy-phenyl]-3-[4-(6-methyl-pyridin-3- |
| | yl)naphthalen-1-yl]urea |
| 166 | N-acetyl-N-(5-tert-butyl-2-methoxy-3-{3-[4-(6-morpholin-4- |
| | ylmethyl-pyridin-3-yl)-naphthalen-1-yl]-ureido}-phenyl)-acetamide |
| 167 | 1-[6-tert-butyl-4-methyl-3-oxo-3,4-dihydro-2H-benzo[1,4]oxazin-8- |
| | yl]-3-[4-(6-morpholin-4-ylmethyl-pyridin-3-yl)naphthalen-1-yl]urea |
| 168 | 1-[5-tert-butyl-2-ethoxy-phenyl]-3-[4-(6-morpholin-4-yl-methyl- |
| | pyridin-3-yl)naphthalen-1-yl]urea |
| 169 | 1-[5-tert-butyl-2-isopropoxy-phenyl]-3-[4-(6-morpholin-4-yl-methyl- |
| | pyridin-3-yl)naphthalen-1-yl]urea |
| 170 | 1-[5-tert-butyl-2-imidazol-1-yl-phenyl]-3-[4-(6-morpholin-4-yl- |
| | methyl-pyridin-3-yl)naphthalen-1-yl]urea |
| 171 | 1-[5-tert-butyl-3-ethylamino-2-methoxy-phenyl]-3-[4-(6-morpholin- |
| | 4-yl-methyl-pyridin-3-yl)naphthalen-1-yl]urea |
| 172 | 1-[5-tert-butyl-2-methoxy-3-{3-[4-(6-morpholin-4-ylmethyl-pyridin- |
| | 3-yl)-naphthalen-1-yl]-ureido}-phenyl)-bis(methanesulfon)amide |
| 173 | 1-[5-tert-butyl-2-(1-methyl-1H-pyrazol-4-yl)-phenyl]-3-[4-(6- |
| | morpholin-4-yl-methyl-pyridin-3-yl)naphthalen-1-yl]urea |
| 174 | 1-[2-methanesulfinyl-5-trifluoromethyl-phenyl]-3-[4-(6-morpholin-4- |
| | yl-methyl-pyridin-3-yl)naphthalen-1-yl]urea |
| 175 | 1-[4-(6-{[bis(2-methoxy-ethyl)-amino]-methyl}-pyridin-3-yl- |
| | naphthalen-1-yl]-3-[5-tert-2-methoxy-phenyl]urea |
| 176 | N-[1-(5-{4-[3-(5-tert-butyl-2-methoxy-phenyl)-ureido]-naphthalen-1- |
| | yl}-pyridin-2-ylmethyl)-pyrrolidin-3-yl]-acetamide |
| 177 | 1-[1-acetyl-3,3-dimethyl-2,3-dihydro-1H-indol-5-yl]-3-[4-(6-morpholin-4-yl- |
| | methyl-pyridin-3-yl)naphthalen-1-yl]urea |
| 178 | 1-(5-tert-butyl-2-methoxy-3-{3-[4-(6-morpholin-4-ylmethyl-pyridin- |
| | 3-yl)-naphthalen-1-yl]-ureido}-phenyl)-propionamide |
| 179 | 1-[5-tert-butyl-2-methyl-benzooxazol-7-yl]-3-[4-(6-morpholin-4-yl- |
| | methyl-pyridin-3-yl)naphthalen-1-yl]urea |
| 180 | 1-[3-trifluoromethanesulfonyl-phenyl]-3-[4-(6-morpholin-4-yl- |
| | methyl-pyridin-3-yl)naphthalen-1-yl]urea |
| 181 | N-(5-tert-butyl-2-methoxy-3-{3-{4-(6-morpholin-4-ylmethyl-pyridin-3-yl)- |
| | naphthalen-1-yl]-ureido}-phenyl)-isobutyramide |
| 182 | 2-(4-tert-butyl-2-{3-[4-(6-morpholin-4-ylmethyl-pyridin-3-yl)- |
| | naphthalen-1-yl]-ureido}-phenoxy)-acetamide |
| 183 | 1-[5-tert-butyl-2-oxo-2,3-dihydro-benzooxazol-7-yl]-3-[4-(6- |
| | morpholin-4-yl-methyl-pyridin-3-yl)naphthalen-1-yl]urea |
| 184 | 1-[5-tert-butyl-3-cyano-2-methoxy-phenyl]-3-[4-(6-morpholin-4-yl- |
| | methyl-pyridin-3-yl)naphthalene-1-yl]urea |
| 185 | 1-[5-tert-butyl-benzooxazol-7-yl]-3-[4-(6-morpholin-4-yl-methyl- |
| | pyridin-3-yl)naphthalen-1-yl]urea |
| 186 | 5-tert-butyl-2-methoxy-{3-[4-(6-morpholin-4-ylmethyl-pyridin-3-yl)- |
| | naphthalen-1-yl]-ureido}-phenyl)-benzenesulfonamide |
| 187 | Ethanesulfonic acid (5-tert-butyl-2-methoxy-3-{3-[4-(6-morpholin-4- |
| | ylmethyl-pyridin-3-yl)-naphthalen-1-yl]-ure3ido}-phenyl)-amide |
| 188 | 1-[5-tert-butyl-2-methoxy-phenyl]-3-[4-(2-morpholin-4-yl-methyl- |
| | pyrimidin-5-yl)naphthalen-1-yl]urea |
| 189 | 1-[5-tert-butyl-2-methylsulfanyl-phenyl]-3-[4-(6-morpholin-4-yl- |
| | methylpyridin-3-yl)naphthalen-1-yl]urea |
| 190 | 1-[5-tert-butyl-2-methoxy-pyridin-3-yl]-3-[4-(6-morpholin-4-yl- |
| | methyl6-pyridin-3-yl)naphthalene-1-yl]urea |
| 191 | 2,2,2-trifluoroethanesulfonic acid (5-tert-butyl-2-methoxy-3-{3-[4-(6- |
| | morpholin-4-ylmethyl-pyridin-3-yl)-naphthalen-1-yl]-ureido}- |
| | phenyl)-amide |
| 192 | N-(5-{4-[3-(5-tert-butyl-2-methyl-phenyl)-ureido]-naphthalen-1-yl}- |
| | pyrazin-2-yl)-methanesulfonamide |
| 193 | 1-[5-tert-butyl-2-methoxy-phenyl]-3-[4-(6-{[bis-(2-cyano-ethyl)-amino]-methyl |
| | }-pyridin-3-yl)naphthalen-1-yl]urea |
| 194 | 1-[5-tert-butyl-2-methoxy-phenyl]-3-{4-[6-(4-methyl-piperazin-1- |
| | ylmethyl)pyridin-3-yl]naphthalene-1-yl}urea |
| 195 | 1-[5-tert-butyl-2-methoxy-phenyl]-3-[4-(6-thiomorpholin-4-yl- |
| | methyl-pyridin-3-yl)naphthalene-1-yl]urea |
| 196 | 1-[5-tert-butyl-2-methoxy-phenyl]-3-{4-[6-(2,6-dimethyl-piperidin-1- |
| | ylmethyl)-pyridin-3-yl]naphthalen-1-yl}urea |
| 197 | 1-[5-tert-butyl-2-methoxy-phenyl]-3-{4-[6-(1-oxo-tetrahydropyran-4- |
| | ylamino)-pyridin-3-yl]naphthalene-1-yl}urea |
| 198 | 1-[5-tert-butyl-2-methoxy-phenyl]-3-{4-[6-(tetrahydropyran-4- |
| | ylamino)-pyridin-3-yl]naphthalene-1-yl}urea |
| 199 | 1-[5-tert-butyl-2-methoxy-phenyl]-3-[4-[6-{[(2-cyano-ethyl)- |
| | tetrahydro-furan-2-ylmethyl)-amino]-methyl}-pyridin-3- |
| | yl)naphthalen-1-yl}urea |
| 200 | 1-[5-tert-butyl-2-methoxy-phenyl]-3-{4-[6-(2-methoxymethyl- |
| | morpholin-4-ylmethyl)-pyridin-3-yl]naphthalen-1-yl}urea |
| 201 | 1-[5-tert-butyl-2-methoxy-phenyl]-3-{4-[6-(2-methyl-3-oxo- |
| | piperazin-1-ylmethyl)-pyridin-3-yl]naphthalen-1-yl}urea |
| 202 | 1-(5-{4-[3-(5-tert-butyl-2-methoxy-phenyl)-ureido]-naphthalen-1-yl}- |
| | pyridin-2-ylmethyl)-piperidine-3-carboxylic acid amide |
| 203 | 1-[5-tert-butyl-2-methoxy-phenyl]-3-{4-[6-(1-oxo-thiomorpholin-4-ylmethyl)- |
| | pyridin-3-yl]naphthalen-1-yl}urea |
| 204 | 1-(3,3-dimethyl-2-oxo-2,3-dihydro-1H-indol-5-yl)-3-[4-(6- |
| | morpholin-4-ylmethyl-pyridin-3-yl)-naphthalen-1-yl]urea |
| 205 | 1-[5-tert-butyl-2-methoxy-phenyl]-3-{4-[6-(3-oxo-piperazin-1- |
| | ylmethyl)-pyridin-3-yl]naphthalen-1-yl}urea |
| 206 | 1-[5-tert-butyl-2-methoxy-phenyl]-3-(4-{6-[(tetrahydro-furan-3- |
| | ylamino)methyl]-pyridin-3-yl}naphthalen-1-yl}urea |
| 207 | 1-[5-tert-butyl-2-methoxy-phenyl]-3-[4-(6-{[(2-cyano-ethyl)-pyridin- |
| | 3-ylmethyl-amino]-methyl}-pyridin-3-yl)naphthalen-1-yl]urea |
| 208 | 1-[5-tert-butyl-2-methoxy-phenyl]-3-{4-[6-(2-oxa-5-aza- |
| | bicyclo[2.2.1]hept-5-ylmethyl)-pyridin-3-yl]naphthalen-1-yl}urea |
| 209 | 1-[5-tert-butyl-2-methoxy-phenyl]-3-{4-[6-(2,6-dimethyl-morpholin- |
| | 4-ylmethyl)pyridin-3-yl]naphthalen-1-yl}urea |
| 210 | 1-[5-tert-butyl-2-methoxy-phenyl]-3-(4-{6-[4-(3-methoxy-phenyl)-1- |
| | piperazin-1-ylmethyl]-pyridin-3-yl}naphthalen-1-yl)urea |
| 211 | 1-[5-tert-butyl-2-methoxy-phenyl]-3-{4-[6-(morpholine-4-carbonyl)- |
| | pyridin-3-yl]naphthalen-1-yl}urea |
| 212 | 1-[5-tert-butyl-2-methoxy-phenyl]-3-[4-(5-morpholin-4-ylmethyl- |
| | pyrazin-2-yl)-naphthalen-1-yl}urea |
| 213 | 1-[6-tert-butyl-3-oxo-3,4-dihydro-2H-benzo[1,4]oxazin-8-yl]-3-[4-(6- |
| | morpholin-4-ylmethyl-pyridin-3-yl)naphthalen-1-yl]urea |
| 214 | 1-[3-amino-5-tert-butyl-2-methoxy-phenyl]-3-[4-(6-morpholin-4-yl- |
| | methyl-pyridin-3-yl)naphthalen-1-yl]urea |
| 215 | N-(5-{4-[3-(5-tert-butyl-2-methoxy-phenyl)-ureido]-naphthalen-1- |
| | yl}pyridin-2-yl)-acetamide |
| 216 | N-(5-tert-butyl-2-methoxy-3-{3-[4-(6-morpholin-4-ylmethyl-pyridin- |
| | 3-yl)-naphthalen-1-yl]-ureido}-phenyl)-N-methyl-acetamide |
| 217 | N-(5-tert-butyl-2-methoxy-3-{3-[4-(6-morpholin-4-ylmethyl-pyridin- |
| | 3-yl)-naphthalen-1-yl]-ureido}-phenyl)-2,2,2-trifluoro-acetamide |
| 218 | 1-[5-tert-butyl-2-methoxy-phenyl]-3-{4-[6-(pyridin-3-yloxy)-pyridin-3-yl] |
| | naphthalen-1-yl}urea |
| 219 | [4-(6-morpholin-4-ylmethyl-pyridin-3-yl)-naphthalen-1-yl]-carbamic |
| | acid 3-tert-butyl-phenyl ester |
| 220 | N-(5-tert-butyl-2-methoxy-3-{3-[4-(6-morpholin-4-ylmethyl-pyridin- |
| | 3-yl)-naphthalen-1-yl]-ureido}-phenyl)-methanesulfonamide |
| 221 | 1-[2-carbomethoxy-5-tert-butyl-3-thienyl]-3-[4-(2-morpholin-4-yl- |
| | ethoxy)naphthalen-1-yl]urea |
| 222 | 1-[2-methylcarbamoyl-5-tert-butyl-3-thienyl]-3-[4-(2-morpholin-4-yl- |
| | ethoxy)naphthalen-1-yl]urea |
| 223 | 1-[2-methylcarbamoyl-5-tert-butyl-3-thienyl]-3-[4-(2-(trans-2,6- |
| | dimethylmorpholin-4-yl)ethoxy)naphthalen-1-yl]urea |
| 224 | 1-[2-methylcarbamoyl-5-tert-butyl-3-thienyl]-3-[4-(2-(2- |
| | dimethylaminomethyldimethylmorpholin-4-yl)ethoxy)naphthalen-1- |
| | yl]urea |
| 225 | 1-[2-carbomethoxy-5-tert-butyl-3-thienyl]-3-[4-(2-(1- |
| | oxothiomorpholin-4-yl)ethoxy)naphthalen-1-yl]urea |
| 226 | 1-[2-methylcarbamoyl-5-tert-butyl-3-thienyl]-3-[4-(2-(1- |
| | oxothiomorpholin-4-yl)ethoxy)naphthalen-1-yl]urea |
| 227 | 1-[3-carbomethoxy-5-tert-butyl-2-thienyl]-3-[4-(2-(1- |
| | oxothiomorpholin-4-yl)ethoxy)naphthalen-1-yl]urea |
| 228 | 1-[2-carbomethoxy-5-tert-butyl-3-thienyl]-3-[4-(2-(tetrahydropyran- |
| | 4-yl)ethoxy)naphthalen-1-yl]urea |
| 229 | 1-[2-methylcarbamoyl-5-tert-butyl-3-thienyl]-3-[4-(2- |
| | (tetrahydropyran-4-yl)ethoxy)naphthalen-1-yl]urea |
| 230 | 1-[3-carbomethoxy-5-tert-butyl-2-thienyl]-3-[4-(2-(tetrahydropyran-4-yl)ethoxy) |
| | naphthalen-1-yl]urea |
| 231 | 1-[2-carbomethoxy-5-tert-butyl-3-thienyl]-3-[4-(2-(1-oxo- |
| | tetrahydrothiophen-3-yl)ethoxy)naphthalen-1-yl]urea |
| 232 | 1-[2-methylcarbamoyl-5-tert-butyl-3-thienyl]-3-[4-(2-(1-oxo- |
| | tetrahydrothiophen-3-yl)ethoxy)naphthalen-1-yl]urea |
| 233 | 1-[2-carbomethoxy-5-tert-butyl-3-thienyl]-3-[4-(pyridin-4-yl- |
| | methoxy)naphthalen-1-yl]urea |
| 234 | 1-[2-methylcarbamoyl-5-tert-butyl-3-thienyl]-3-[4-(pyridin-4- |
| | yl)methoxy)naphthalen-1-yl]urea |
| 235 | 1-[2-carbomethoxy-5-tert-butyl-3-thienyl]-3-[4-(2-imidazol-1-yl- |
| | ethoxy)naphthalen-1-y1]urea |
| 236 | 1-[2-carbomethoxy-5-tert-butyl-3-thienyl]-3-[4-(2-(3,4- |
| | dimethoxyphenyl)-ethoxy)naphthalen-1-yl]urea |
| 237 | 1-[2-methylcarbamoyl-5-tert-butyl-3-thienyl]-3-[4-(2-(3,4- |
| | dimethoxyphenyl)ethoxy)naphthalen-1-yl]urea |
| 238 | 1-[2-carbomethoxy-5-tert-butyl-3-pyrrolyl]-3-[4-(2-morpholin-4-yl- |
| | ethoxy)naphthalen-1-yl]urea |
| 239 | 1-[2-methylcarbamoyl-5-tert-butyl-3-pyrrolyl]-3-[4-(2-morpholin-4- |
| | yl-ethoxy)naphthalen-1-yl]urea |
| 240 | 1-[1-methyl-2-carbomethoxy-5-tert-butyl-3-pyrrolyl]-3-[4-(2- |
| | morpholin-4-yl-ethoxy)naphthalen-1-yl]urea |
| 241 | 1-[2-carbomethoxy-5-tert-butyl-3-pyrrolyl]-3-[4-(2-(1- |
| | oxothiomorpholin-4-yl)ethoxy)naphthalen-1-yl]urea |
| 242 | 1-[2-methylcarbamoyl-5-tert-butyl-3-pyrrolyl]-3-[4-(2-(1- |
| | oxothiomorpholin-4-yl)ethoxy)naphthalen-1-yl]urea |
| 243 | 1-[1-methyl-2-carbomethoxy-5-tert-butyl-3-pyrrolyl]-3-[4-(2-(1- |
| | oxothiomorpholin-4-yl)ethoxy)naphthalen-1-yl]urea |
| 244 | 1-[2-carbomethoxy-5-tert-butyl-3-pyrrolyl]-3-[4-(2-(tetrahydropyran- |
| | 4-yl)ethoxy)naphthalen-1-yl]urea |
| 245 | 1-[2-methylcarbamoyl-5-tert-butyl-3-pyrrolyl]-3-[4-(2- |
| | (tetrahydropyran-4-yl)ethoxy)naphthalen-1-yl]urea |
| 246 | 1-[2-carbomethoxy-5-tert-butyl-3-pyrrolyl]-3-[4-(2-(1-oxo- |
| | tetrahydrothiophen-3-yl)ethoxy)naphthalen-1-yl]urea |
| 247 | 1-[2-methylcarbamoyl-5-tert-butyl-3-pyrrolyl]-3-[4-(2-(1-oxo- |
| | tetrahydrothiophen-3-yl)ethoxy)naphthalen-1-yl]urea |
| 248 | 1-[1-methyl-2-carbomethoxy-5-tert-butyl-3-pyrrolyl]-3-[4-(2-(1- |
| | tetrahydrothiophen-3-yl)ethoxy)naphthalen-1-yl]urea |
| 249 | 1-[2-carbomethoxy-5-tert-butyl-3-pyrrolyl]-3-[4-(pyridin-4-yl- |
| | methoxy)naphthalen-1-yl]urea |
| 250 | 1-[2-methylcarbamoyl-5-tert-butyl-3-pyrrolyl]-3-[4-(pyridin-4-yl- |
| | methoxy)naphthalen-1-yl]urea |
| 251 | 1-[2-carbomethoxy-5-tert-butyl-3-pyrrolyl]-3-[4-(pyridin-4-yl- |
| | ethoxy)naphthalen-1-yl]urea |
| 252 | 1-[2-carbomethoxy-5-tert-butyl-3-pyrrolyl]-3-[4-(2-imidazol-1-yl- |
| | ethoxy)naphthalen-1-yl]urea |
| 253 | 1-[2-carbomethoxy-5-tert-butyl-3-pyrrolyl]-3-[4-(2-(3,4- |
| | dimethoxyphenyl)ethoxy)naphthalen-1-yl]urea |
| 254 | 1-[2-methylcarbamoyl-5-tert-butyl-3-pyrrolyl]-3-[4-(2-(3,4- |
| | dimethoxyphenyl)ethoxy)naphthalen-1-yl]urea |
| 255 | 1-(5-tert-butyl-2-methoxyphenyl)-3-[4-(2-methoxy-pyridin-4-yl-oxy)- |
| | naphthalen-1-yl]urea |
| 256 | 1-(5-tert-butyl-2-methoxyphenyl)-3-[4-(2-methyl-pyridin-4-yl-oxy)- |
| | naphthalen-1-yl]urea |
| 257 | 1-(5-tert-butyl-2,3-dimethoxyphenyl)-3-[4-(2-methoxy-pyridin-4-yl- |
| | oxy)-naphthalen-1-yl]urea |
| 258 | 5-tert-butyl-2-methoxy-3-{3-(pyridin-4-yl-oxy)naphthalen-1- |
| | yl]ureido}benzamide |
| 259 | Morpholine carboxylic acid (5-tert-butyl-2-methoxy-3-{3-[-(pyridin-4-yloxy) |
| | naphthalen-1-yl}ureido}phenyl)amide |
| 260 | N-(5-tert-butyl-2-methoxy-3-{3-[-(pyridin4-yl-oxy)naphthalen-1- |
| | yl]ureido}phenyl)acetamide |
| 261 | 3-(5-tert-butyl-2-methoxy-3-{3-[-(pyridin-4-yl-oxy)naphthalen-1- |
| | yl]ureido}phenyl)-1,1-dimethylurea |
| 262 | 1-(5-tert-butyl-2,3-dimethoxy-phenyl)-3-[4-(2-amino-pyridin-4- |
| | yloxy)-naphthalen-1-yl]urea |
| 263 | 1-(5-tert-butyl-2,3-dimethoxyphenyl)-3-[4-(2-methylamino-pyridin-4- |
| | yloxy]-naphthalen-1-yl}urea |
| 264 | 1-(5-tert-butyl-2,3-dimethoxyphenyl)-3-{4-[2-(1-phenyl-ethylamino)- |
| | pyridin-4-yloxy]-naphthalen-1-yl}urea |
| 265 | 1-(5-tert-butyl-2-methoxy-pyridin-3-yl)-3-[4-(2-amino-pyridin-4- |
| | yloxy)-naphthalen-1-yl]urea |
| 266 | 1-(5-tert-butyl-2-methoxy-pyridin-3-yl)-3-[4-(2-methylamino- |
| | pyridin-4-yloxy]-naphthalen-1-yl}urea |
| 267 | 1-(5-tert-butyl-2-methoxy-pyridin-3-yl)-3-{4-[2-(1-phenyl- |
| | ethylamino)-pyridin-4-yloxy]-naphthalen-1-yl}urea |
| 268 | 1-(5-tert-butyl-2-methyl-phenyl)-3-[4-(2-aminopyridin-4-yl-oxy)- |
| | naphthalen-1-yl]urea |
| 269 | 1-(5-tert-butyl-2-morpholin-4-yl-phenyl)-3-[4-(2-morpholin-4-yl- |
| | ethoxy)-naphthalen-1-yl]urea |
| 270 | 1-(5-tert-butyl-2-methoxyphenyl)-3-[4-(2-methylpyridin-4-yl-oxy)- |
| | naphthalen-1-yl]urea |
| 271 | 1-(5-tert-butyl-2,3-dimethoxyphenyl)-3-[4-(2-methoxypyridin-4-yl- |
| | oxy)-naphthalen-1-yl]urea |
| 272 | 5-tert-butyl-2-methoxy-3-{3-[-(pyridin-4-yl-oxy)naphthalen-1- |
| | yl]ureido}benzamide |
| 273 | Morpholine-4-carboxylic acid (5-tert-butyl-2-methoxy-3-{3-[- |
| | (pyridin-4-yl-oxy)naphthalen-1-yl]ureido}phenyl)amide |
| 274 | N-(5-tert-butyl-2-methoxy-3-{3-[-(pyridin-4-yl-oxy)naphthalen-1- |
| | yl]}ureido}phenyl)acetamide |
| 275 | 3-(5-tert-butyl-2-methoxy-3-{3-[-(pyridin-4-yl-oxy)naphthalen-1- |
| | yl]ureido}phenyl)-1,1-dimethylurea |
| 276 | 1-(5-tert-butyl-2,3-dimethoxy-phenyl)-3-[4-(2-amino-pyridin-4- |
| | yloxy)-naphyhalen-1-yl]urea |
| 277 | 1-(5-tert-butyl-2,3-dimethoxy-phenyl)-3-[4-(2-methylamino-pyridin- |
| | 4-yloxy)-naphthalen-1-yl]urea |
| 278 | 1-(5-tert-butyl-2,3-dimethoxy-phenyl)-3-[4-(2-phenyl-ethylamino- |
| | pyridin-4-yloxy)-naphthalen-1-yl]urea |
| 279 | 1-(5-tert-butyl-2-methoxy-pyridin-3-yl)-3-[4-(2-amino-pyridin-4- |
| | yloxy)-naphthalen-1-yl]urea |
| 280 | 1-(5-tert-butyl-2-methoxy-pyridin-3-yl)-3-[4-(2-methylamino- |
| | pyridin-4-yloxy)-naphthalen-1-yl]urea |
| 281 | 1-(5-tert-butyl-2-methoxy-pyridin-3-yl)-3-{4-[2-(1-phenyl- |
| | ethylamino)-pyridin-4-yloxy)-naphthalen-1-yl]urea |
| 282 | 1-(5-tert-butyl-2-methyl-phenyl)-3-[4-(2-aminopyridin-4-yloxy)- |
| | naphthalen-1-yl]urea |
| 283 | 1-(5-tert-butyl-2-morpholin-4-yl-phenyl)-3-[4-(2-morpholin-4-yl- |
| | ethoxy)-naphthalen-1-yl]urea |

b) salts of the ureas within the above group,
c) prodrugs of the ureas within the above group and
d) isolated stereoisomers of the ureas within the above group.

2. A method as in claim 1 wherein the disease that is treated or prevented is a KDR-mediated disorder.

3. A method as in claim 1 wherein the disease that is treated or prevented is a Flk-1 mediated disorder.

4. A method of regulating tyrosine kinase signal transduction comprising administering to a human or other mammal one or more compounds described in tables 1-5 above.

5. The method of claim 1, wherein said disease is cancer.

6. A method for treating or preventing a raf mediated disease in a human or other mammal comprising administering to a human or other mammal in need thereof one or more compounds selected from the group consisting of:
a) ureas numbered 1-283 in claim 1
b) salts of the ureas numbered 1-283 in claim 1,
c) prodrugs of the ureas numbered 1-283 in claim 1 and
d) isolated stereoisomers of the ureas numbered 1-283 in claim 1.

7. A method as in claim 6 wherein the raf mediated disease is cancer.

8. A method for treating and preventing hyper-proliferative disorders comprising administering to a human or other mammal one or more compounds selected from the group consisting of:
a) ureas numbered 1-283 in claim 1
b) salts of the ureas numbered 1-283 in claim 1,
c) prodrugs of the ureas numbered 1-283 in claim 1 and
d) isolated stereoisomers of the ureas numbered 1-283 in claim 1.

9. A method as in claim 8 wherein the hyper-proliferative disorder that is treated is a solid cancer, carcinoma of the lungs, carcinoma of the pancreas, carcinoma of the thyroid, carcinoma of the bladder, carcinoma of the colon, a myeloid disorder, or an adenoma.

10. A method as in claim 9 wherein the hyper-proliferative disorder that is treated is a raf kinase-mediated cancer.

11. A method as in claim 8 wherein the hyper-proliferative disorder that is treated is human colon, gastric, lung, pancreatic, ovarian, prostate, leukemia, melanoma, hepatocellular, renal, head and neck, glioma, or mammary cancer.

12. A method as in claim 8 wherein at least one additional anti-proliferative agent is administered with the one or more administered compounds defined in claim 1, either simultaneously in the same pharmaceutical composition or separately, in separate pharmaceutical compositions.

13 A method as in claim 12 wherein the additional anti-proliferative agent is selected from the group consisting of asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, vindesine,
aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, oxaliplatin, pentostatin, N-phosphonoacetyl-L-aspartate (PALA), plicamycin, semustine, teniposide, testosterone propionate, thiotepa, trimethylmelamine, uridine, vinorelbine, epothilone, gemcitabine, taxotere, BCNU, CCNU, DTIC, 5-fluorouarcil, herceptin and actinomycin D.

14. A method as in claim 13 wherein the one or more administered compounds and the additional anti-proliferative agent are administered separately in separate formulations.

15. A method as in claim 1 wherein at least one cytotoxic agent or cytostatic chemotherapeutic agent is administered with the one or more administered compounds defined in claim 1, either simultaneously in the same pharmaceutical composition or separately, in separate pharmaceutical compositions,
wherein the cytotoxic agent or cytostatic chemotherapeutic agent is selected from the group consisting of DNA topoisomerase I and II inhibitors, DNA intercalators, alkylating agents, microtubule disruptors, hormone and growth factor receptor agonists or antagonists, other kinase inhibitors and antimetabolites.

16. A method as in claim 14 wherein the hyper-proliferative disorder that is treated is human pancreatic, lung, colon, ovarian, prostate, leukemia, melanoma, hepatocellular, renal, head and neck, glioma, or mammary carcinoma.

17. A kit comprising a separate dose of the one or more compounds defined in claim 1 and a separate dose of an additional anti-proliferative agent.

18. A method for the treatment of a disease mediated by the VEGF induced signal transduction pathway within a human or other mammal, comprising administering to a human or other mammal with such a disease, one or more compounds selected from the group consisting of:
a) one or more ureas selected from the group consisting of

| | |
|---|---|
| 1 | 1-[5-tert-butyl-2-(2-methylpyridin-5-yl)-2H-pyrazol-3-yl]-3-(4- |
| | chlorophenyl)urea |
| 2 | 1-(5-tert-butyl-2-p-tolyl-2H-pyrazol-3-yl)-3-(4-methoxynaphthalen-1- |
| | yl)urea |
| 3 | 1-[5-tert-butyl-2-(3,4-dimethylphenyl)-2H-pyrazol-3-yl]-3-(4- |
| | fluorophenyl)urea |
| 4 | 1-[5-tert-butyl-2-(pyridin-3-yl)-2H-pyrazol-3-yl]-3-(4- |
| | cyanonaphthalen-1-yl)urea |
| 5 | 1-[5-tert-butyl-2-p-tolyl-2H-pyrazol-3-yl]-3-[4-(2-morpholin-4- |
| | ylethoxy)naphthalen-1-yl]-urea |
| 6 | 1-[5-tert-butyl-2-p-tolyl-2H-pyrazol-3-yl]-3-[4-(2-(cis-2,6- |
| | dimethylmorpholin-4-yl)ethoxy)naphthalen-1-yl]-urea |
| 7 | 1-[5-tert-butyl-2-p-tolyl-2H-pyrazol-3-yl]-3-[4-(2-(trans-2,6- |
| | dimethylmorpholin-4-yl)ethoxy)naphthalen-1-yl]-urea |
| 8 | 1-[5-tert-butyl-2-p-tolyl-2H-pyrazol-3-yl]-3-[4-(2-(2- |
| | (methoxymethyl)morpholin-4-yl)ethoxy)naphthalen-1-yl]-urea |
| 9 | 1-[5-tert-butyl-2-p-tolyl-2H-pyrazol-3-yl]-3-[4-(2-(morpholin-4-yl)-2- |
| | oxoethoxy)naphthalen-1-yl]-urea |
| 10 | 1-[5-tert-butyl-2-p-tolyl-2H-pyrazol-3-yl]-3-[4-(2-(morpholin-4-yl)-2- |
| | methylethoxy)naphthalen-1-yl]-urea |
| 11 | 1-[5-tert-butyl-2-p-tolyl-2H-pyrazol-3-yl]-3-[4-(2-(morpholin-4-yl)-1- |
| | methylethoxy)naphthalen-1-yl]-urea |
| 12 | 1-[5-tert-butyl-2-p-tolyl-2H-pyrazol-3-yl]-3-[4-(2-thiomorpholin-4- |
| | ylethoxy)naphthalen-1-yl]-urea |
| 13 | 1-[5-tert-butyl-2-p-tolyl-2H-pyrazol-3-yl]-3-[4-(2-(1- |
| | oxothiomorpholin-4-yl)ethoxy)naphthalen-1-yl]-urea |
| 14 | 1-[5-tert-butyl-2-p-tolyl-2H-pyrazol-3-yl]-3-[4-(2-morpholin-4-yl- |
| | ethoxy)-3-methylnaphthalen-1-yl]-urea |
| 15 | 1-[5-tert-butyl-2-p-tolyl-2H-pyrazol-3-yl]-3-[4-(2-(morpholin-4-yl- |
| | carbonyloxo)ethoxy)naphthalen-1-yl]-urea |
| 16 | 1-[5-tert-butyl-2-p-tolyl-2H-pyrazol-3-yl]-3-[4-(2-(tetrahydropyran-4-yl)ethoxy) |
| | naphthalen-1-yl]-urea |
| 17 | 1-[5-tert-butyl-2-p-tolyl-2H-pyrazol-3-yl]-3-[4-(2-(1-oxo- |
| | tetrahydrothiophen-3-yl)ethoxy)naphthalen-1-yl]-urea |
| 18 | 1-[5-tert-butyl-2-p-tolyl-2H-pyrazol-3-yl]-3-[4-(3-morpholin-4-yl- |
| | propyl)naphthalen-1-yl]-urea |
| 19 | 1-[5-tert-butyl-2-p-tolyl-2H-pyrazol-3-yl]-3-[4-(3-morpholin-4-yl- |
| | methyl)naphthalen-1-yl]-urea |
| 20 | 1-[5-tert-butyl-2-p-tolyl-2H-pyrazol-3-yl]-3-[4-(2-pyridin-4-yl- |
| | ethyl)naphthalen-1-yl]-urea |
| 21 | 1-[5-tert-butyl-2-p-tolyl-2H-pyrazol-3-yl]-3-[4-(3-(morpholin-4- |
| | yl)propyn-1-yl)naphthalen-1-yl]-urea |
| 22 | 1-[5-tert-butyl-2-p-tolyl-2H-pyrazol-3-yl]-3-[4-(3-(tetrahydropyran-2- |
| | yl-oxy)propyn-1-yl)naphthalen-1-yl]-urea |
| 23 | 1-[5-tert-butyl-2-p-tolyl-2H-pyrazol-3-yl]-3-[4-(3-(tetrahydropyran-2- |
| | yl-oxy)butyn-1-yl)naphthalen-1-yl]-urea |
| 24 | 1-[5-tert-butyl-2-p-tolyl-2H-pyrazol-3-yl]-3-[4-(3-(piperidin-1- |
| | yl)propyn-1-yl)naphthalen-1-yl]-urea |
| 25 | 1-[5-tert-butyl-2-p-tolyl-2H-pyrazol-3-yl]-3-[4-(3-(2- |
| | methoxymethylmorpholin-4-yl)propyn-1-yl)naphthalen-1-yl]-urea |
| 26 | 1-[5-tert-butyl-2-p-tolyl-2H-pyrazol-3-yl]-3-[4-(pyridin-4-yl- |
| | methoxy)naphthalen-1-yl]-urea |
| 27 | 1-[5-tert-butyl-2-p-tolyl-2H-pyrazol-3-yl]-3-[4-(2-pyridin-4-yl- |
| | ethoxy)naphthalen-1-yl]-urea |
| 28 | 1-[5-tert-butyl-2-p-tolyl-2H-pyrazol-3-yl]-3-[4-(3-pyridin-4-yl- |
| | propoxy)naphthalen-1-yl]-urea |
| 29 | 1-[5-tert-butyl-2-p-tolyl-2H-pyrazol-3-yl]-3-[4-(2-imidazol-1-yl- |
| | ethoxy)naphthalen-1-yl]-urea |
| 30 | 1-[5-tert-butyl-2-p-tolyl-2H-pyrazol-3-yl]-3-[4-(2-(3,4- |
| | dimethoxyphenyl)-ethoxy)naphthalen-1-yl]-urea |
| 31 | 1-[5-tert-butyl-2-p-tolyl-2H-pyrazol-3-yl]-3-[4-(pyridin-4-yl- |
| | methylamino)naphthalen-1-yl]-urea |
| 32 | 1-[5-isopropyl-2-phenyl-2H-pyrazol-3-yl]-3-[4-(2-morpholin-4-yl- |
| | ethoxy)naphthalen-1-yl]-urea |
| 33 | 1-[5-cyclohexyl-2-phenyl-2H-pyrazol-3-yl]-3-[4-(2-morpholin-4-yl- |
| | ethoxy)naphthalen-1-yl]-urea |
| 34 | 1-[5-(2,2,2-trifluoroethyl)-2-phenyl-2H-pyrazol-3-yl]-3-[4-(2- |
| | morpholin-4-yl-ethoxy)naphthalen-1-yl]-urea |
| 35 | 1-[5-(1-methylcycloprop-1-yl)-2-phenyl-2H-pyrazol-3-yl]-3-[4-(2- |
| | morpholin-4-yl-ethoxy)naphthalen-1-yl]-urea |
| 36 | 1-[5-(1-methylcyclohex-1-yl)-2-phenyl-2H-pyrazol-3-yl]-3-[4-(2-morpholin-4-yl- |
| | ethoxy)naphthalen-1-yl]-urea |
| 37 | 1-[5-tert-butyl-2-methyl-2H-pyrazol-3-yl]-3-[4-(2-morpholin-4-yl- |
| | ethoxy)naphthalen-1-yl]-urea |
| 38 | 1-[5-tert-butyl-2-(4-chlorophenyl)-2H-pyrazol-3-yl]-3-[4-(2- |
| | morpholin-4-yl-ethoxy)naphthalen-1-yl]-urea |
| 39 | 1-[5-tert-butyl-2-butyl-2H-pyrazol-3-yl]-3-[4-(2-morpholin-4-yl- |
| | ethoxy)naphthalen-1-yl]-urea |
| 40 | 1-[5-tert-butyl-2-(4-methyl-3-carbamylphenyl)-2H-pyrazol-3-yl]-3- |
| | [4-(2-morpholin-4-yl-ethoxy)naphthalene-1-yl]-urea |
| 41 | 1-[5-tert-butyl-2-(4-methyl-3-morpholin-4-yl)methylphenyl)-2H- |
| | pyrazol-3-yl]-3-[4-(2-morpholin-4-yl-ethoxy)naphthalen-1-yl]-urea |
| 42 | 1-[5-tert-butyl-2-(4-methyl-3-dimethylaminomethylphenyl)-2H- |
| | pyrazol-3-yl]-3-[4-(2-morpholin-4-yl-ethoxy)naphthalen-1-yl]-urea |
| 43 | 1-[5-tert-butyl-2-(3-dimethylaminomethylphenyl)-2H-pyrazol-3-yl]- |
| | 3-[4-(2-morpholin-4-yl-ethoxy)naphthalen-1-yl]-urea |
| 44 | 1-[5-tert-butyl-2-(2-chloropyridin-5-yl)-2H-pyrazol-3-yl]-3-[4-(2- |
| | morpholin-4-yl-ethoxy)naphthalen-1-yl]-urea |
| 45 | 1-[5-tert-butyl-2-(2-methylpyridin-5-yl)-2H-pyrazol-3-yl]-3-[4-(2- |
| | morpholin-4-yl-ethoxy)naphthalen-1-yl]-urea |
| 46 | 1-[5-tert-butyl-2-(2-methoxypyridin-5-yl)-2H-pyrazol-3-yl]-3-[4-(2- |
| | morpholin-4-yl-ethoxy)naphthalen-1-yl]-urea |
| 47 | 1-[5-tert-butyl-2-(pyridin-3-yl)-2H-pyrazol-3-yl]-3-[4-(2-morpholin- |
| | 4-yl-ethoxy)naphthalen-1-yl]-urea |
| 48 | 1-[5-tert-butyl-2-(2-methylpyridin-5-yl)-2H-pyrazol-3-yl]-3-[4-(2- |
| | pyridin-4-yl-ethoxy)naphthalen-1-yl]-urea |
| 49 | 1-[5-tert-butyl-2-(2-methylpyridin-5-yl)-2H-pyrazol-3-yl]-3-[4-(2- |
| | (trans-2,6-dimethylmorpholin-4-yl)ethoxy)naphthalen-1-yl]-urea |
| 50 | 1-[5-tert-butyl-2-(2-methylpyridin-5-yl)-2H-pyrazol-3-yl]-3-[4-(3- |
| | morpholin-4-yl-propyn-1-yl)naphthalene-1-yl]-urea |
| 51 | 1-[5-(2-hydroxy-1,1-dimethyl-ethyl)-2-p-tolyl-2H-pyrazol-3-yl]-3-[4- |
| | (2-morpholin-4-yl-ethoxy)naphthalen-1-yl]-urea |
| 52 | 1-[5-tert-butyl-2-(3-hydroxy-4-methyl-phenyl)-2H-pyrazol-3-yl]-3-[4- |
| | (2-morpholin-4-yl-ethoxy)naphthalene-1-yl]-urea |
| 53 | 1-[5-tert-butyl-2-(4-hydroxymethyl-phenyl)-2H-pyrazol-3-yl]-3-[4-(2- |
| | morpholin-4-yl-ethoxy)naphthalen-1-yl]-urea |
| 54 | 1-[5-tert-butyl-2-p-tolyl-2H-pyrazol-3-yl]-3-{4-[2-(3-oxo-morpholin- |
| | 4-yl)-ethoxy]naphthalen-1-yl}-urea |
| 55 | 1-[5-tert-butyl-2-p-tolyl-2H-pyrazol-3-yl]-3-{4-[2-(4-hydroxy- |
| | morpholin-4-yl)-ethoxy]naphthalen-1-yl}-urea |
| 56 | 1-[5-(2-hydroxy-1,1-dimethyl-ethyl)-2-(6-methyl-pyridin-3-yl)-2H- |
| | pyrazol-3-yl]-3-[4-(2-morpholin-4-yl-ethoxy)naphthalen-1-yl]-urea |
| 57 | 1-[5-tert-butyl-2-(1-hydroxy-6-methyl-pyridin-3-yl)-2H-pyrazol-3- |
| | yl]-3-[4-(2-morpholin-4-yl-ethoxy)naphthalen-1-yl]-urea |
| 58 | 1-[5-tert-butyl-2-(6-methyl-pyridin-3-yl)-2H-pyrazol-3-yl]-3-{4-[2-(4-hydroxy- |
| | morpholin-4-yl)-ethoxy]naphthalen-1-yl}-urea |
| 59 | 1-[5-(2-hydroxy-1,1-dimethyl-ethyl)-2-(6-methyl-pyridin-3-yl)-2H- |
| | pyrazol-3-yl]-3-[4-(2-pyridin-4-yl-ethoxy)naphthalen-1-yl]-urea |
| 60 | 1-[5-tert-butyl-2-(6-methyl-pyridin-3-yl)-2H-pyrazol-3-yl]-3-[4-(2- |
| | hydroxy-2-pyridin-4-yl-ethoxy)naphthalen-1-yl]-urea |
| 61 | 1-[5-tert-butyl-2-(6-methyl-pyridin-3-yl)-2H-pyrazol-3-yl]-3-{4-[2- |
| | (1-hydroxy-2-pyridin-4-yl)-ethoxy]naphthalen-1-yl}-urea |
| 62 | 1-[5-(2-hydroxy-1,1-dimethyl-ethyl)-2-p-tolyl-2H-pyrazol-3-yl]-3-{4-[2-(1-oxo- |
| | thiomorpholin-4-yl)-ethoxy]naphthalen-1-yl}-urea |
| 63 | 1-[5-tert-butyl-2-(4-hydroxymethyl-phenyl)-2H-pyrazol-3-yl]-3-{4- |
| | [2-(1-oxo-thiomorpholin-4-yl)-ethoxy]naphthalen-1-yl}-urea |
| 64 | 1-(5-tert-butyl-2-p-tolyl-2H-pyrazol-3-yl]-3-{4-[2-(1,3-dioxo- |
| | thiomorpholin-4-yl)-ethoxy]naphthalene-1-yl}-urea |
| 65 | 1-[5-(2-hydroxy-1,1-dimethyl-ethyl)-2-methyl-2H-pyrazol-3-yl]-3-[4- |
| | (2-morpholin-4-yl-ethoxy)-naphthalen-1-yl]-urea |
| 66 | 1-[5-tert-butyl-2-methyl-2H-pyrazol-3-yl]-3-{4-[2-(4-hydroxy- |
| | morpholin-4-yl)-ethoxy]-naphthalen-1-yl}-urea |
| 67 | 1-[5-tert-butyl-2-p-tolyl-2H-pyrazolyl-3-yl]-3-[4-(4-(morpholin-4-yl- |
| | methyl)phenyl)naphthalen-1-yl]urea |
| 68 | 1-[5-tert-butyl-2-p-tolyl-2H-pyrazolyl-3-yl]-3-[4-(4-(2-(morpholin-4- |
| | yl)ethyl)phenyl)naphthalen-1-yl]urea |
| 69 | 1-[5-tert-butyl-2-p-tolyl-2H-pyrazolyl-3-yl]-3-[4-(3-(morpholin-4-yl- |
| | methyl)phenyl)naphthalen-1-yl]urea |
| 70 | 1-[5-tert-butyl-2-p-tolyl-2H-pyrazolyl-3-yl]-3-[4-(6-(morpholin-4- |
| | ylmethyl-pyridin-3-yl)naphthalen-1-yl]urea |
| 71 | 1-[5-tert-butyl-2-p-tolyl-2H-pyrazolyl-3-yl]-3-[4-(5-(morpholin-4- |
| | ylmethyl-pyridin-2-yl)naphthalen-1-yl]urea |
| 72 | 1-[5-tert-butyl-2-p-tolyl-2H-pyrazolyl-3-yl]-3-[4-(5-(morpholin-4- |
| | ylmethyl-fur-2-yl)naphthalen-1-yl]urea |
| 73 | 1-[5-tert-butyl-2-(6-methyl-pyridin-3-yl)-2H-pyrazolyl-3-yl]-3-[4-(6- |
| | (morpholin-4-ylmethyl-pyridin-3-yl)naphthalen-1-yl]urea |
| 74 | 1-[5-tert-butyl-2-methyl-2H-pyrazolyl-3-yl]-3-[4-(6-morpholin-4- |
| | ylmethyl-pyridin-3-yl)naphthalen-1-yl]urea |
| 75 | 1-[5-tert-butyl-2-p-tolyl-2H-pyrazolyl-3-yl]-3-[4-(5-(morpholin-4-yl- |
| | methyl)pyridin-2-yl)naphthalen-1-yl]urea |
| 76 | 1-[5-tert-butyl-2-p-tolyl-2H-pyrazolyl-3-yl]-3-[4-(6-(morpholin-4-yl- |
| | methyl)pyridin-3-yl)naphthalen-1-yl]urea |
| 77 | 1-[5-tert-butyl-2-(6-methyl-pyridin-3-yl)-2H-pyrazolyl-3-yl]-3-[4-(3- |
| | (2-(pyridin-2-yl)ethylamino)cyclohexenyl)-naphthalen-1-yl]urea |
| 78 | 1-[5-tert-butyl-2-p-tolyl-2H-pyrazolyl-3-yl]-3-[4-(4-(pyridin-3-yl- |
| | methylaminomethyl)phenyl)naphthalene-1-yl]urea |
| 79 | 1-[5-tert-butyl-2-(6-methyl-pyridin-3-yl)-2H-pyrazolyl-3-yl]-3-[4-(4- |
| | (morpholin-4-yl-methyl)phenyl)naphthalen-1-yl]urea |
| 80 | 1-[5-tert-butyl-2-(6-methyl-pyridin-3-yl)-2H-pyrazolyl-3-yl]-3-[4-(6- |
| | (4-(hydroxybutylamino)pyridin-3-yl)-naphthalen-1-yl]urea |
| 81 | 1-[5-tert-butyl-2-(4-methyl-3-carbamylphenyl)-2H-pyrazolyl-3-yl]-3- |
| | [4-(6-(morpholin-4-yl-methyl)pyridin-3-yl)naphthalen-1-yl]urea |
| 82 | 1-[5-tert-butyl-2-(6-methyl-pyridin-3-yl)-2H-pyrazolyl-3-yl]-3-[4-(4- |
| | (3-hydroxypiuperidin-1-yl-methyl)phenyl)naphthalen-1-yl]urea |
| 83 | 1-[5-tert-butyl-2-(6-methyl-pyridin-3-yl)-2H-pyrazolyl-3-yl]-3-[4-(4- |
| | (4-hydroxymorpholin-4-yl-methyl)phenyl)naphthalen-1-yl]urea |
| 84 | 1-[5-tert-butyl-2-(6-methyl-pyridin-3-yl)-2H-pyrazolyl-3-yl]-3-[4-(3-(morpholin- |
| | 4-yl-methyl)cyclohexenyl)naphthalen-1-yl]urea |
| 85 | 1-[5-tert-butyl-2-(6-methyl-pyridin-3-yl)-2H-pyrazolyl-3-yl]-3-[4-(4- |
| | (tetrahydrofuran-3-yl-methyl)-3-hydroxyphenyl)naphthalen-1-yl]urea |
| 86 | 1-[5-tert-butyl-2-(6-methyl-pyridin-3-yl)-2H-pyrazolyl-3-yl]-3-[4-(4- |
| | (N,N-di-(2-methoxethyl)aminomethyl)phenyl)naphthalen-1-yl]urea |
| 87 | 1-[5-tert-butyl-2-(6-methyl-pyridin-3-yl)-2H-pyrazolyl-3-yl]-3-[4-(6- |
| | (cyanopropoxy)pyridin-3-yl)naphthalen-1-yl]urea |
| 88 | 1-[5-tert-butyl-2-(6-methyl-pyridin-3-yl)-2H-pyrazolyl-3-yl]-3-[4-(4- |
| | morpholin-4-yl-methyl-piperidinyl)naphthalen-1-yl]urea |
| 89 | 1-[5-tert-butyl-2-(6-methyl-pyridin-3-yl)-2H-pyrazolyl-3-yl]-3-[4-(4- |
| | (N,N-di-(2-cyanoethyl)aminomethyl)phenyl)naphthalen-1-yl]urea |
| 90 | 1-[5-tert-butyl-2-(6-methyl-pyridin-3-yl)-2H-pyrazolyl-3-yl]-3-[4-(4- |
| | (furan-2-yl-methyl)-3-hydroxyphenyl)naphthalen-1-yl]urea |
| 91 | 1-[5-tert-butyl-2-(6-methyl-pyridin-3-yl)-2H-pyrazolyl-3-yl]-3-[4-(4- |
| | (thiomorpholin-4-yl-methyl)phenyl)naphthalen-1-yl]urea |
| 92 | 1-[5-tert-butyl-2-(6-methyl-pyridin-3-yl)-2H-pyrazolyl-3-yl]-3-[4-(4- |
| | (3-carboxamidopiperidin-1-yl-methyl)phenyl)naphthalen-1-yl]urea |
| 93 | 1-[5-tert-butyl-2-(6-methyl-pyridin-3-yl)-2H-pyrazolyl-3-yl]-3-[4-(4- |
| | (2-methyl-3-oxo-piperzin-1-yl-methyl)phenyl)naphthalen-1-yl]urea |
| 94 | 1-[5-tert-butyl-2-(2-methylpyrimidin-5-yl)-2H-pyrazolyl-3-yl]-3-[4- |
| | (6-(morpholin-4-yl-methyl)pyridin-3-yl)naphthalen-1-yl]urea |
| 95 | 1-[5-tert-butyl-2-(6-methyl-pyridin-3-yl)-2H-pyrazolyl-3-yl]-3-[4-(6- |
| | (4-hydroxybutyloxy)pyridin-3-yl)naphthalen-1-yl]urea |
| 96 | 1-[3-tert-butyl-1′H-[1,4′]bipyrazol-5-yl]-3-[4-(6-(morpholin-4-ylmethyl)pyridin- |
| | 3-yl)naphthalen-1-yl]urea |
| 97 | 1-[5-tert-butyl-2-2H-pyrazolyl-3-yl]-3-[4-(6-(tetrahydrothiopyran-4- |
| | yl-amino)pyridin-3-yl)naphthalen-1-yl]urea |
| 98 | 1-[5-tert-butyl-2-(2-cyanoethyl)-2H-pyrazolyl-3-yl]-3-[4-(6- |
| | (morpholin-4-yl-methyl)pyridin-3-yl)naphthalen-1-yl]urea |
| 99 | 1-[5-tert-butyl-2-(6-methyl-pyridin-3-yl)-2H-pyrazolyl-3-yl]-3-[4-(6- |
| | (2,6-dim ethylmorpholin-4-yl-methyl)pyridin-3-yl)naphthalen-1- |
| | yl]urea |
| 100 | 1-[5-tert-butyl-2-(2-methoxypyridin-5-yl)-2H-pyrazolyl-3-yl]-3-[4-(6- |
| | (morpholin-4-yl-methyl)pyridin-3-yl)naphthalen-1-yl]urea |
| 101 | 1-[5-tert-butyl-2-(2-aminopyridin-5-yl)-2H-pyrazolyl-3-yl]-3-[4-(6- |
| | (morpholin-4-yl-methyl)pyridin-3-yl)naphthalen-1-yl]urea |
| 102 | 1-[5-tert-butyl-2-(6-methyl-pyridin-3-yl)-2H-pyrazolyl-3-yl]-3-[4-(6- |
| | (morpholin-4-yl-4-carbonyl)pyridin-3-yl)naphthalen-1-yl]urea |
| 103 | 1-[5-tert-butyl-2-(6-methyl-pyridin-3-yl)-2H-pyrazolyl-3-yl]-3-[4-(6- |
| | (2-oxa-5-aza-bicyclo[2.2.1]hept-5-yl-methyl)pyridin-3-yl)naphthalen- |
| | 1-yl]urea |
| 104 | 1-[5-tert-butyl-2-(6-methyl-pyridin-3-yl)-2H-pyrazolyl-3-yl]-3-[4-(4- |
| | (N-(2-cyanoethyl)-N-(pyridin-3- |
| | ylmethyl)aminomethyl)phenyl)naphthalen-1-yl]urea |
| 105 | 1-[5-tert-butyl-2-(6-methyl-pyridin-3-yl)-2H-pyrazolyl-3-yl]-3-[4-(4- |
| | (N-(2-cyanoethyl)-N-(tetrahydrofuran-2-yl- |
| | methyl)aminomethyl)phenyl)naphthalen-1-yl]urea |
| 106 | 1-[5-tert-butyl-2-(6-methyl-pyridin-3-yl)-2H-pyrazolyl-3-yl]-3-[4-(6-(morpholin- |
| | 4-yl-methyl)-4-methoxypyridin-3-yl)naphthalen-1-yl]urea |
| 107 | 1-[5-tert-butyl-2-(6-methyl-pyridin-3-yl)-2H-pyrazolyl-3-yl]-3-[4-(6- |
| | (1-morpholin-4-yl-propyl)pyridin-3-yl)naphthalen-1-yl]urea |
| 108 | 1-[3-tert-butyl-1′-methyl-1′H-[1,4′]bipyrazol-5-yl]-3-[4-(6- |
| | (morpholin-4-yl-methyl)pyridin-3-yl)naphthalen-1-yl]urea |
| 109 | 1-[5-tert-butyl-2-(6-methyl-pyridin-3-yl)-2H-pyrazolyl-3-yl]-3-[4-(6- |
| | (1-oxo-tetrahydrothiopyran-4-yl-amino)pyridin-3-yl)naphthalen-1- |
| | yl]urea |
| 110 | 1-[5-tert-butyl-2-(6-methyl-pyridin-3-yl)-2H-pyrazolyl-3-yl]-3-[4-(6- |
| | (tetrahydropyran-4-yl-amino)pyridin-3-yl)naphthalen-1-yl]urea |
| 111 | 1-[5-tert-butyl-2-(6-methyl-pyridin-3-yl)-2H-pyrazolyl-3-yl]-3-[4-(5- |
| | (tetrahydrothiopyran-4-yl-aminbo)pyrazin-2-yl)naphthalen-1-yl]urea |
| 112 | 1-[5-tert-butyl-2-(6-methyl-pyridin-3-yl)-2H-pyrazolyl-3-yl]-3-[4(6- |
| | (metrhylcarbonylamino)pyridin-3-yl)naphthalen-1-yl]urea |
| 113 | 1-[5-tert-butyl-1′-(3-methylsulfanylpropyl)-1′H-[1,4′bipyrazol-5- |
| | yl]-3-[4-(6-(morpholin-4-yl-methyl)pyridin-3-yl)naphthalen-1-yl]urea |
| 114 | 1-[5-tert-butyl-2-(2-methylpyrimidin-5-yl)-2H-pyrazolyl-3-yl]-3-[4- |
| | (6-(1-oxo-thiomorpholin-4-yl-methyl)pyridin-3-yl)naphthalen-1- |
| | yl]urea |
| 115 | 1-[5-tert-butyl-2-(2-methylpyrimidin-5-yl)-2H-pyrazolyl-3-yl]-3-[4- |
| | (6-(tetrahydropyran-4-ylamino)pyridin-3-yl)naphthalen-1-yl]urea |
| 116 | 1-[5-tert-butyl-2-(2-methylthiopyrimidin-5-yl)-2H-pyrazolyl-3-yl]-3- |
| | 4-(6-morpholin-4-yl-methyl)pyridin-3-yl)naphthalen-1-yl]urea |
| 117 | 1-[5-tert-butyl-2-(2-aminopyrimidin-5-yl)-2H-pyrazolyl-3-yl]3-[4- |
| | (6-(morpholin-4-yl-methyl)pyridin-3-yl)naphthalen-1-yl]urea |
| 118 | 1-[3-tert-butyl-1′-methyl-1′H-[1,4;]bipyrazol-5-yl]-3-[4-(6- |
| | (morpholin-4-yl-methyl)phenyl-)naphthalen-1-yl]urea |
| 119 | 1-[5-tert-butyl-2-p-tolyl-2H-pyrazolyl-3-yl]-3-[4-(6-(1-oxo- |
| | tetrahydrothiopyran-4-yl-amino)pyridin-3-yl)naphthalen-1-yl]urea |
| 120 | 1-[5-tert-butyl-2-p-tolyl-2H-pyrazolyl-3-yl]-3-[4-(6-(thiomorpholin- |
| | 4-yl-methyl)pyridin-3-yl)naphthalen-1-yl]urea |
| 121 | 1-[5-tert-butyl-2-p-tolyl-2H-pyrazolyl-3-yl]-3-[4-(2-(morpholin-4-yl- |
| | carbonyl)pyrimidin-5-yl)naphthalen-1-yl]urea |
| 122 | 1-[5-tert-butyl-2-p-tolyl-2H-pyrazolyl-3-yl]-3-[4-(4-(morpholin-4-yl- |
| | methyl)pyrimidin-5-yl)naphthalen-1-yl]urea |
| 123 | 1-[5-tert-butyl-2-p-tolyl-2H-pyrazolyl-3-yl]-3-[4-(6-(1-oxo- |
| | thiomorpholin-4-yl-methyl)pyridin-3-yl)naphthalen-1-yl]urea |
| 124 | 1-[5-tert-butyl-2-(2-methylpyrimidin-5-yl)-2H-pyrazolyl-3-yl]-3-[4- |
| | (2-(morpholin-4-yl-methyl)pyrimidin-5-yl)naphthalen-1-yl]urea |
| 125 | 1-[2-tert-butyl-5-methyl-pyridin-4-yl]-3-[4-(6-(morpholin-4-yl- |
| | methyl-pyridin-3-yl)naphthalen-1-yl]urea |
| 126 | 1-[3-tert-butyl-phenyl]-3-[4-(6-(morpholin-4-yl-methyl)pyridin-3- |
| | yl)naphthalen-1-yl]urea |
| 127 | 1-[4-methyl-biphenyl-3-yl]-3-[4-(6-(morpholin-4-yl-methyl-pyridin- |
| | 3-yl)naphthalen-1-yl]urea |
| 128 | 1-[4-tert-butyl-biphenyl-2-yl]-3-[4-(6-morpholin-4-yl-methyl-pyridin- |
| | 3-yl)naphthalen-1-yl]urea |
| 129 | 1-[5-isopropyl-2-methyl-phenyl]-3-[4-(6-morpholin-4-yl-methyl- |
| | pyridin-3-yl)naphthalen-1-yl]urea |
| 130 | 1-[5-sec-butyl-2-methoxy-phenyl]-3-[4-(6-(morpholin-4-yl-methyl- |
| | pyridin-3-yl)naphthalen-1-yl]urea |
| 131 | 1-[5-tert-butyl-2-methoxymethyl-phenyl]-3-[4-(6-(morpholin-4-yl- |
| | methyl-pyridin-3-yl)naphthalen-1-yl]urea |
| 132 | 1-[5-tert-butyl-2-methoxy-phenyl]-3-[4-(6-(morpholin-4-yl-methyl- |
| | pyridin-3-yl)naphthalen-1-yl]urea |
| 133 | 1-[5-tert-butyl-2-mewthyl-phenyl]-3-{6-[(3-(methoxy-propyl)- |
| | methyl-amino]-pyridin-3-yl}naphthalen-1-yl]urea |
| 134 | 1-[5-tert-butyl-2-methyl-phenyl]-3-[4-(6-morpholin-4-yl-methyl- |
| | pyridin-3-yl)naphthalen-1-yl]urea |
| 135 | 1-[5-tert-butyl-2-methyl-pyridin-3-yl]-3-[4-(6-morpholin-4-yl- |
| | methyl-pyridin-3-yl)naphthalen-1-yl]urea |
| 136 | 1-[5-(1,1-dimethylpropyl)-2-methoxy-phenyl]-3-[4-(6-morpholin-4- |
| | yl-methyl-pyridin-3-yl)naphthalen-1-yl]urea |
| 137 | 1-[5-tert-butyl-2-(1H-pyrazol-4-yl)-phenyl]-3-[4-(6-morpholin-4-yl- |
| | methyl-pyridin-3-yl)naphthalen-1-yl]urea |
| 138 | 1-[5-tert-butyl-2-(2-methyl-pyrimidin-5-yl)-phenyl]-3-[4-(6- |
| | morpholin-4-yl-methyl-pyridin-3-yl)naphthalen-1-yl]urea |
| 139 | 1-[5-tert-butyl-2-(3-hydroxy-propyl)-phenyl]-3-[4-(6-morpholin-4-yl-methyl- |
| | pyridin-3-yl)naphthalen-1-yl]urea |
| 140 | 1-[5-tert-butyl-2-(morpholine-4-carbonyl)-phenyl]-3-[4-(6- |
| | morpholin-4-yl-methyl-pyridin-3-yl)naphthalen-1-yl]urea |
| 141 | 1-[5-tert-butyl-2-methoxy-3-{3-[4-(6-morpholin-4-ylmethyl-pyridin- |
| | 3-yl)-naphthalen-1-yl]-ureido}-phenyl)-acetamide |
| 142 | 1-[3-methyl-naphthalen-2-yl]-3-[4-(6-morpholin-4-yl-methyl-pyridin- |
| | 3-yl)naphthalen-1-yl]urea |
| 143 | 1-[5-tert-butyl-2-methoxy-3-{3-[4-(6-morpholin-4-ylmethyl-pyridin- |
| | 3-yl)-naphthalen-1-yl]-urido}-phenyl)-acetamide |
| 144 | 1-[5-tert-butyl-3-(2,3-dihydroxy-propyl)-2-hydroxy-phenyl]-3-[4-(6- |
| | morpholin-4-yl-methyl-pyridin-3-yl)naphthalen-1-yl]urea |
| 145 | 1-[2,3-dimethyl-1H-indol-5-yl]-3-[4-(6-morpholin-4-yl-methyl- |
| | pyridin-3-yl)naphthalen-1-yl]urea |
| 146 | 1-{5-tert-butyl-2-methyl-3-[3-(tetrahydro-pyran-2-yloxy)-prop-1- |
| | ynyl]-phenyl}-3-[4-(6-morpholin-4-yl-methyl-pyridin-3- |
| | yl)naphthalen-1-yl]urea |
| 147 | 1-[2-methoxy-5-trifluoromethyl-phenyl]-3-[4-(6-morpholin-4-yl- |
| | methyl-pyrimidin-3-yl)naphthalen-1-yl]urea |
| 148 | 1-[5-(2,2-dimethyl-propionyl)-2-methyl-phenyl]-3-[4-(6-morpholin- |
| | 4-yl-methyl-pyridin-3-yl)naphthalen-1-yl]urea |
| 149 | 1-[5-tert-butyl-3-(3-hydroxy-prop-1-ynyl)-2-methyl-phenyl]-3-[4-(6- |
| | morpholin-4-yl-methyl-pyridin-3-yl)naphthalen-1-yl]urea |
| 150 | 1-[5-tert-butyl-2-(3-hydroxy-propyn-1-ynyl)-phenyl]-3-[4-(6- |
| | morpholin-4-yl-methyl-pyridin-3-yl)naphthalen-1-yl]urea |
| 151 | 1-[5-tert-butyl-3-(2,2-dimethyl-[1,3]dioxolan-4-ylmethyl)-2- |
| | methoxy-phenyl]-3-[4-(6-morpholin-4-yl-methyl-pyridin-3- |
| | yl)naphthalen-1-yl]urea |
| 152 | 1-[5-tert-butyl-3-(2,3-dihydroxy-propyl)-2-methoxy-phenyl]-3-[4-(6- |
| | morpholin-4-yl-methyl-pyridin-3-yl)naphthalen-1-yl]urea |
| 153 | 1-[5-tert-butyoxy-2-methoxy-phenyl]-3-[4-(6-morpholin-4-yl-methyl- |
| | pyridin-3-yl)naphthalen-1-yl]urea |
| 154 | 1-[5-(1-cyano-cyclopropyl)-2-methoxy-phenyl]-3-[4-(6-morpholin-4- |
| | yl-methyl-pyridin-3-yl)naphthalen-1-yl]urea |
| 155 | 1-[5-tert-butyl-3-(2-diethylamino-ethyl)-2-methoxy-phenyl]-3-[4-(6- |
| | morpholin-4-yl-methyl-pyridin-3-yl)naphthalen-1-yl]urea |
| 156 | 1-[5-tert-butyl-2-methoxy-phenyl]-3-[4-(6-[1,3]dioxolan-2-yl- |
| | pyridin-3-yl)naphthalen-1-yl]urea |
| 157 | 1-[5-tert-butyl-2-pyrrolidin-1-yl-phenyl]-3-[4-(6-morpholin-4-yl- |
| | methyl-pyridin-3-yl)naphthalen-1-yl]urea |
| 158 | 1-[5-tert-butyl-2-dimethylamino-phenyl]-3-[4-(6-morpholin-4-yl- |
| | methyl-pyridin-3-yl)naphthalen-1-yl]urea |
| 159 | 1-[5-tert-butyl-2-propoxy-phenyl]-3-[4-(6-morpholin-4-yl-methyl- |
| | pyridin-3-yl)naphthalen-1-yl]urea |
| 160 | 1-[5-tert-butyl-2-methoxy-phenyl]-3-[4-(6-hydroxymethyl-pyridin-3- |
| | yl)naphthalen-1-yl]urea |
| 161 | 1-[5-tert-butyl-2-methoxy-phenyl]-3-{4-[6-(2,6-dimethyl-morpholin- |
| | 4-yl-methyl)pyridin-3-yl]naphthalene-l-yl}urea |
| 162 | 1-[5-cyclohexyl-2-methoxy-phenyl]-3-[4-(6-morpholin-4-yl-methyl- |
| | pyridin-3-yl)naphthalen-1-yl]urea |
| 163 | 1-[2,4-dimethoxy-5-trifluoromethyl-phenyl]-3-[4-(6-morpholin-4-yl- |
| | methyl-pyridin-3-yl)naphthalen-1-yl]urea |
| 164 | 1-[5-tert-butyl-2-methoxy-3-nitro-phenyl]-3-[4-(6-morpholin-4-yl- |
| | methyl-pyridin-3-yl)naphthalen-1-yl]urea |
| 165 | 1-[3-amino-5-tert-butyl-2-methoxy-phenyl]-3-[4-(6-methyl-pyridin-3- |
| | yl)naphthalen-1-yl]urea |
| 166 | N-acetyl-N-(5-tert-butyl-2-methoxy-3-{3-[4-(6-morpholin-4- |
| | ylmethyl-pyridin-3-yl)-naphthalen-1-yl]-ureido}-phenyl)-acetamide |
| 167 | 1-[6-tert-butyl-4-methyl-3-oxo-3,4-dihydro-2H-benzo[1,4]oxazin-8- |
| | yl]-3-[4-(6-morpholin-4-ylmethyl-pyridin-3-yl)naphthalen-1-yl]urea |
| 168 | 1-[5-tert-butyl-2-ethoxy-phenyl]-3-[4-(6-morpholin-4-yl-methyl- |
| | pyridin-3-yl)naphthalen-1-yl]urea |
| 169 | 1-[5-tert-butyl-2-isopropoxy-phenyl]-3-[4-(6-morpholin-4-yl-methyl- |
| | pyridin-3-yl)naphthalen-1-yl]urea |
| 170 | 1-[5-tert-butyl-2-imidazol-1-yl-phenyl]-3-[4-(6-morpholin-4-yl- |
| | methyl-pyridin-3-yl)naphthalen-1-yl]urea |
| 171 | 1-[5-tert-butyl-3-ethylamino-2-methoxy-phenyl]-3-[4-(6-morpholin- |
| | 4-yl-methyl-pyridin-3-yl)naphthalene-1-yl]urea |
| 172 | 1-[5-tert-butyl-2-methoxy-3-{3-[4-(6-morpholin-4-ylmethyl-pyridin- |
| | 3-yl)-naphthalen-1-yl]-ureido}-phenyl)-bis(methanesulfon)amide |
| 173 | 1-[5-tert-butyl-2-(1-methyl-1H-pyrazol-4-yl)-phenyl]-3-[4-(6- |
| | morpholin-4-yl-methyl-pyridin-3-yl)naphthalen-1-yl]urea |
| 174 | 1-[2-methanesulfinyl-5-trifluoromethyl-phenyl]-3-[4-(6-morpholin-4- |
| | yl-methyl-pyridin-3-yl)naphthalen-1-yl]urea |
| 175 | 1-[4-(6-{[bis(2-methoxy-ethyl)-amino]-methyl}-pyridin-3-yl- |
| | naphthalen-1-yl]-3-[5-tert-2-methoxy-phenyl]urea |
| 176 | N-[1-(5-{4-[3-(5-tert-butyl-2-methoxy-phenyl)-ureido]-naphthalen-1-yl}-pyridin- |
| | 2-ylmethyl)-pyrrolidin-3-yl]-acetamide |
| 177 | 1-[1-acetyl-3,3-dimethyl-2,3-dihydro-1H-indol-5-yl]-3-[4-(6- |
| | morpholin-4-yl-methyl-pyridin-3-yl)naphthalen-1-yl]urea |
| 178 | 1-(5-tert-butyl-2-methoxy-3-{3-[4-(6-morpholin-4-ylmethyl-pyridin- |
| | 3-yl)-naphthalen-1-yl]-ureido}-phenyl)-propionamide |
| 179 | 1-[5-tert-butyl-2-methyl-benzooxazol-7-yl]-3-[4-(6-morpholin-4-yl- |
| | methyl-pyridin-3-yl)naphthalen-1-yl]urea |
| 180 | 1-[3-trifluoromethanesulfonyl-phenyl]-3-[4-(6-morpholin-4-yl- |
| | methyl-pyridin-3-yl)naphthalen-1-yl]urea |
| 181 | N-(5-tert-butyl-2-methoxy-3-{3-{4-(6-morpholin-4-ylmethyl-pyridin- |
| | 3-yl)-naphthalen-1-yl]-ureido}-phenyl)-isobutyramide |
| 182 | 2-(4-tert-butyl-2-{3-[4-(6-morpholin-4-ylmethyl-pyridin-3-yl)- |
| | naphthalen-1-yl]-ureido}-phenoxy)-acetamide |
| 183 | 1-[5-tert-butyl-2-oxo-2,3-dihydro-benzooxazol-7-yl]-3-[4-(6- |
| | morpholin-4-yl-methyl-pyridin-3-yl)naphthalen-1-yl]urea |
| 184 | 1-[5-tert-butyl-3-cyano-2-methoxy-phenyl]-3-[4-(6-morpholin-4-yl- |
| | methyl-pyridin-3-yl)naphthalen-1-yl]urea |
| 185 | 1-[5-tert-butyl-benzooxazol-7-yl]-3-[4-(6-morpholin-4-yl-methyl- |
| | pyridin-3-yl)naphthalen-1-yl]urea |
| 186 | 5-tert-butyl-2-methoxy-{3-[4-(6-morpholin-4-ylmethyl-pyridin-3-yl)- |
| | naphthalen-1-yl]-ureido}-phenyl)-benzenesulfonamide |
| 187 | Ethanesulfonic acid (5-tert-butyl-2-methoxy-3-{3-[4-(6-morpholin-4- |
| | ylmethyl-pyridin-3-yl)-naphthalen-1-yl]-ure3ido}-phenyl)-amide |
| 188 | 1-[5-tert-butyl-2-methoxy-phenyl]-3-[4-(2-morpholin-4-yl-methyl- |
| | pyrimidin-5-yl)naphthalen-1-yl]urea |
| 189 | 1-[5-tert-butyl-2-methylsulfanyl-phenyl]-3-[4-(6-morpholin-4-yl- |
| | methylpyridin-3-yl)naphthalen-1-yl]urea |
| 190 | 1-[5-tert-butyl-2-methoxy-pyridin-3-yl]-3-[4-(6-morpholin-4-yl- |
| | methyl6-pyridin-3-yl)naphthalen-1-yl]urea |
| 191 | 2,2,2-trifluoroethanesulfonic acid (5-tert-butyl-2-methoxy-3-{3-[4-(6- |
| | morpholin-4-ylmethyl-pyridin-3-yl)-naphthalen-1-yl]-ureido}- |
| | phenyl)-amide |
| 192 | N-(5-{4-[3-(5-tert-butyl-2-methyl-phenyl)-ureido]-naphthalen-1-yl}- |
| | pyrazin-2-yl)-methanesulfonamide |
| 193 | 1-[5-tert-butyl-2-methoxy-phenyl]-3-[4-(6-{[bis-(2-cyano-ethyl)- |
| | amino]-methyl}-pyridin-3-yl)naphthalen-1-yl]urea |
| 194 | 1-[5-tert-butyl-2-methoxy-phenyl]-3-{4-[6-(4-methyl-piperazin-1- |
| | ylmethyl)pyridin-3-yl]naphthalen-1-yl}urea |
| 195 | 1-[5-tert-butyl-2-methoxy-phenyl]-3-[4-(6-thiomorpholin-4-yl- |
| | methyl-pyridin-3-yl)naphthalen-1-yl]urea |
| 196 | 1-[5-tert-butyl-2-methoxy-phenyl]-3-{4-[6-(2,6-dimethyl-piperidin-1- |
| | ylmethyl)-pyridin-3-yl]naphthalen-1-yl}urea |
| 197 | 1-[5-tert-butyl-2-methoxy-phenyl]-3-{4-[6-(1-oxo-tetrahydropyran-4- |
| | ylamino)-pyridin-3-yl]naphthalen-1-yl}urea |
| 198 | 1-[5-tert-butyl-2-methoxy-phenyl]-3-{4-[6-(tetrahydropyran-4- |
| | ylamino)-pyridin-3-yl]naphthalen-1-yl}urea |
| 199 | 1-[5-tert-butyl-2-methoxy-phenyl]-3-[4-[6-{[(2-cyano-ethyl)- |
| | tetrahydro-furan-2-ylmethyl)-amino]-methyl}-pyridin-3- |
| | yl)naphthalen-1-yl}urea |
| 200 | 1-[5-tert-butyl-2-methoxy-phenyl]-3-{4-[6-(2-methoxymethyl- |
| | morpholin-4-ylmethyl)-pyridin-3-yl]naphthalen-1-yl}urea |
| 201 | 1-[5-tert-butyl-2-methoxy-phenyl]-3-{4-[6-(2-methyl-3-oxo- |
| | piperazin-1-ylmethyl)-pyridin-3-yl]naphthalen-1-yl}urea |
| 202 | 1-(5-{4-[3-(5-tert-butyl-2-methoxy-phenyl)-ureido]-naphthalen-1-yl}- |
| | pyridin-2-ylmethyl)-piperidine-3-carboxylic acid amide |
| 203 | 1-[5-tert-butyl-2-methoxy-phenyl]-3-{4-[6-(1-oxo-thiomorpholin-4- |
| | ylmethyl)-pyridin-3-yl]naphthalene-1-yl}urea |
| 204 | 1-(3,3-dimethyl-2-oxo-2,3-dihydro-1H-indol-5-yl)-3-[4-(6- |
| | morpholin-4-ylmethyl-pyridin-3-yl)-naphthalen-1-yl]urea |
| 205 | 1-[5-tert-butyl-2-methoxy-phenyl]-3-{4-[6-(3-oxo-piperazin-1- |
| | ylmethyl)-pyridin-3-yl]naphthalene-1-yl}urea |
| 206 | 1-[5-tert-butyl-2-methoxy-phenyl]-3-(4-{6-[(tetrahydro-furan-3- |
| | ylamino)methyl]-pyridin-3-yl}naphthalen-1-yl}urea |
| 207 | 1-[5-tert-butyl-2-methoxy-phenyl]-3-[4-(6-{[(2-cyano-ethyl)-pyridin- |
| | 3-ylmethyl-amino]-methyl}-pyridin-3-yl)naphthalen-1-yl]urea |
| 208 | 1-[5-tert-butyl-2-methoxy-phenyl]-3-{4-[6-(2-oxa-5-aza- |
| | bicyclo[2.2.1]hept-5-ylmethyl)-pyridin-3-yl]naphthalen-1-yl}urea |
| 209 | 1-[5-tert-butyl-2-methoxy-phenyl]-3-{4-[6-(2,6-dimethyl-morpholin- |
| | 4-ylmethyl)pyridin-3-yl]naphthalene-1-yl}urea |
| 210 | 1-[5-tert-butyl-2-methoxy-phenyl]-3-(4-{6-[4-(3-methoxy-phenyl)-1- |
| | piperazin-1-ylmethyl]-pyridin-3-yl}naphthalen-1-yl)urea |
| 211 | 1-[5-tert-butyl-2-methoxy-phenyl]-3-{4-[6-(morpholine-4-carbonyl)- |
| | pyridin-3-yl]naphthalen-1-yl}urea |
| 212 | 1-[5-tert-butyl-2-methoxy-phenyl]-3-[4-(5-morpholin-4-ylmethyl- |
| | pyrazin-2-yl)-naphthalen-1-yl}urea |
| 213 | 1-[6-tert-butyl-3-oxo-3,4-dihydro-2H-benzo[1,4]oxazin-8-yl]-3-[4-(6- |
| | morpholin-4-ylmethyl-pyridin-3-yl)naphthalen-1-yl]urea |
| 214 | 1-[3-amino-5-tert-butyl-2-methoxy-phenyl]-3-[4-(6-morpholin-4-yl- |
| | methyl-pyridin-3-yl)naphthalen-1-yl]urea |
| 215 | N-(5-{4-[3-(5-tert-butyl-2-methoxy-phenyl)-ureido]-naphthalen-1- |
| | yl}pyridin-2-yl)-acetamide |
| 216 | N-(5-tert-butyl-2-methoxy-3-{3-[4-(6-morpholin-4-ylmethyl-pyridin- |
| | 3-yl)-naphthalen-1-yl]-ureido}-phenyl)-N-methyl-acetamide |
| 217 | N-(5-tert-butyl-2-methoxy-3-{3-[4-(6-morpholin-4-ylmethyl-pyridin- |
| | 3-yl)-naphthalen-1-yl]-ureido}-phenyl)-2,2,2-trifluoro-acetamide |
| 218 | 1-[5-tert-butyl-2-methoxy-phenyl]-3-{4-[6-(pyridin-3-yloxy)-pyridin- |
| | 3-yl]naphthalen-1-yl}urea |
| 219 | [4-(6-morpholin-4-ylmethyl-pyridin-3-yl)-naphthalen-1-yl]-carbamic |
| | acid 3-tert-butyl-phenyl ester |
| 220 | N-(5-tert-butyl-2-methoxy-3-{3-[4-(6-morpholin-4-ylmethyl-pyridin- |
| | 3-yl)-naphthalen-1-yl]-ureido}-phenyl)-methanesulfonamide |
| 221 | 1-[2-carbomethoxy-5-tert-butyl-3-thienyl]-3-[4-(2-morpholin-4-yl- |
| | ethoxy)naphthalen-1-yl]urea |
| 222 | 1-[2-methylcarbamoyl-5-tert-butyl-3-thienyl]-3-[4-(2-morpholin-4-yl-ethoxy) |
| | naphthalen-1-yl]urea |
| 223 | 1-[2-methylcarbamoyl-5-tert-butyl-3-thienyl]-3-[4-(2-(trans-2,6- |
| | dimethylmorpholin-4-yl)ethoxy)naphthalen-1-yl]urea |
| 224 | 1-[2-methylcarbamoyl-5-tert-butyl-3-thienyl]-3-[4-(2-(2- |
| | dimethylaminomethyldimethylmorpholin-4-yl)ethoxy)naphthalen-1- |
| | yl]urea |
| 225 | 1-[2-carbomethoxy-5-tert-butyl-3-thienyl]-3-[4-(2-(1- |
| | oxothiomorpholin-4-yl)ethoxy)naphthalen-1-yl]urea |
| 226 | 1-[2-methylcarbamoyl-5-tert-butyl-3-thienyl]-3-[4-(2-(1- |
| | oxothiomorpholin-4-yl)ethoxy)naphthalen-1-yl]urea |
| 227 | 1-[3-carbomethoxy-5-tert-butyl-2-thienyl]-3-[4-(2-(1- |
| | oxothiomorpholin-4-yl)ethoxy)naphthalen-1-yl]urea |
| 228 | 1-[2-carbomethoxy-5-tert-butyl-3-thienyl]-3-[4-(2-(tetrahydropyran- |
| | 4-yl)ethoxy)naphthalen-1-yl]urea |
| 229 | 1-[2-methylcarbamoyl-5-tert-butyl-3-thienyl]-3-[4-(2- |
| | (tetrahydropyran-4-yl)ethoxy)naphthalen-1-yl]urea |
| 230 | 1-[3-carbomethoxy-5-tert-butyl-2-thienyl]-3-[4-(2-(tetrahydropyran- |
| | 4-yl)ethoxy)naphthalen-1-yl]urea |
| 231 | 1-[2-carbomethoxy-5-tert-butyl-3-thienyl]-3-[4-(2-(1-oxo- |
| | tetrahydrothiophen-3-yl)ethoxy)naphthalen-1-yl]urea |
| 232 | 1-[2-methylcarbamoyl-5-tert-butyl-3-thienyl]-3-[4-(2-(1-oxo- |
| | tetrahydrothiophen-3-yl)ethoxy)naphthalen-1-yl]urea |
| 233 | 1-[2-carbomethoxy-5-tert-butyl-3-thienyl]-3-[4-(pyridin-4-yl- |
| | methoxy)naphthalen-1-yl]urea |
| 234 | 1-[2-methylcarbamoyl-5-tert-butyl-3-thienyl]-3-[4-(pyridin-4- |
| | yl)methoxy)naphthalen-1-yl]urea |
| 235 | 1-[2-carbomethoxy-5-tert-butyl-3-thienyl]-3-[4-(2-imidazol-1-yl- |
| | ethoxy)naphthalen-1-yl]urea |
| 236 | 1-[2-carbomethoxy-5-tert-butyl-3-thienyl]-3-[4-(2-(3,4- |
| | dimethoxyphenyl)-ethoxy)naphthalen-1-yl]urea |
| 237 | 1-[2-methylcarbamoyl-5-tert-butyl-3-thienyl]-3-[4-(2-(3,4- |
| | dimethoxyphenyl)ethoxy)naphthalen-1-yl]urea |
| 238 | 1-[2-carbomethoxy-5-tert-butyl-3-pyrrolyl]-3-[4-(2-morpholin-4-yl- |
| | ethoxy)naphthalen-1-yl]urea |
| 239 | 1-[2-methylcarbamoyl-5-tert-butyl-3-pyrrolyl]-3-[4-(2-morpholin-4- |
| | yl-ethoxy)naphthalen-1-yl]urea |
| 240 | 1-[1-methyl-2-carbomethoxy-5-tert-butyl-3-pyrrolyl]-3-[4-(2- |
| | morpholin-4-yl-ethoxy)naphthalen-1-yl]urea |
| 241 | 1-[2-carbomethoxy-5-tert-butyl-3-pyrrolyl]-3-[4-(2-(1- |
| | oxothiomorpholin-4-yl)ethoxy)naphthalen-1-yl]urea |
| 242 | 1-[2-methylcarbamoyl-5-tert-butyl-3-pyrroly]-3-[4-(2-(1- |
| | oxothiomorpholin-4-yl)ethoxy)naphthalen-1-yl]urea |
| 243 | 1-[1-methyl-2-carbomethoxy-5-tert-butyl-3-pyrrolyl]-3-[4-(2-(1- |
| | oxothiomorpholin-4-yl)ethoxy)naphthalen-1-yl]urea |
| 244 | 1-[2-carbomethoxy-5-tert-butyl-3-pyrrolyl]-3-[4-(2-(tetrahydropyran- |
| | 4-yl)ethoxy)naphthalen-1-yl]urea |
| 245 | 1-[2-methylcarbamoyl-5-tert-butyl-3-pyrrolyl]-3-[4-(2- |
| | (tetrahydropyran-4-yl)ethoxy)naphthalen-1-yl]urea |
| 246 | 1-[2-carbomethoxy-5-tert-butyl-3-pyrrolyl]-3-[4-(2-(1-oxo- |
| | tetrahydrothiophen-3-yl)ethoxy)naphthalen-1-yl]urea |
| 247 | 1-[2-methylcarbamoyl-5-tert-butyl-3-pyrrolyl]-3-[4-(2-(1-oxo- |
| | tetrahydrothiophen-3-yl)ethoxy)naphthalen-1-yl]urea |
| 248 | 1-[1-methyl-2-carbomethoxy-5-tert-butyl-3-pyrrolyl]-3-[4-(2-(1- |
| | tetrahydrothiophen-3-yl)ethoxy)naphthalen-1-yl]urea |
| 249 | 1-[2-carbomethoxy-5-tert-butyl-3-pyrrolyl]-3-[4-(pyridin-4-yl- |
| | methoxy)naphthalen-1-yl]urea |
| 250 | 1-[2-methylcarbamoyl-5-tert-butyl-3-pyrrolyl]-3-[4-(pyridin-4-yl- |
| | methoxy)naphthalen-1-yl]urea |
| 251 | 1-[2-carbomethoxy-5-tert-butyl-3-pyrrolyl]-3-[4-(pyridin-4-yl- |
| | ethoxy)naphthalen-1-yl]urea |
| 252 | 1-[2-carbomethoxy-5-tert-butyl-3-pyrrolyl]-3-[4-(2-imidazol-1-yl- |
| | ethoxy)naphthalen-1-yl]urea |
| 253 | 1-[2-carbomethoxy-5-tert-butyl-3-pyrrolyl]-3-[4-(2-(3,4- |
| | dimethoxyphenyl)ethoxy)naphthalen-1-yl]urea |
| 254 | 1-[2-methylcarbamoyl-5-tert-butyl-3-pyrrolyl]-3-[4-(2-(3,4- |
| | dimethoxyphenyl)ethoxy)naphthalen-1-yl]urea |
| 255 | 1-(5-tert-butyl-2-methoxyphenyl)-3-[4-(2-methoxy-pyridin-4-yl-oxy)- |
| | naphthalen-1-yl]urea |
| 256 | 1-(5-tert-butyl-2-methoxyphenyl)-3-[4-(2-methyl-pyridin-4-yl-oxy)- |
| | naphthalen-1-yl]urea |
| 257 | 1-(5-tert-butyl-2,3-dimethoxyphenyl)-3-[4-(2-methoxy-pyridin-4-yl- |
| | oxy)-naphthalen-1-yl]urea |
| 258 | 5-tert-butyl-2-methoxy-3-{3-(pyridin-4-yl-oxy)naphthalen-1- |
| | yl]ureido}benzamide |
| 259 | Morpholine carboxylic acid (5-tert-butyl-2-methoxy-3-{3-[-(pyridin- |
| | 4-yloxy)naphthalen-1-yl}ureido}phenyl)amide |
| 260 | N-(5-tert-butyl-2-methoxy-3-{3-[-(pyridin4-yl-oxy)naphthalen-1- |
| | yl]ureido}phenyl)acetamide |
| 261 | 3-(5-tert-butyl-2-methoxy-3-{3-[-(pyridin-4-yl-oxy)naphthalen-1- |
| | yl]ureido}phenyl)-1,1-dimethylurea |
| 262 | 1-(5-tert-butyl-2,3-dimethoxy-phenyl)-3-[4-(2-amino-pyridin-4- |
| | yloxy)-naphthalen-1-yl]urea |
| 263 | 1-(5-tert-butyl-2,3-dimethoxyphenyl)-3-[4-(2-methylamino-pyridin-4- |
| | yloxy]-naphthalen-1-yl}urea |
| 264 | 1-(5-tert-butyl-2,3-dimethoxyphenyl)-3-{4-[2-(1-phenyl-ethylamino)- |
| | pyridin-4-yloxy]-naphthalen-1-yl}urea |
| 265 | 1-(5-tert-butyl-2-methoxy-pyridin-3-yl)-3-[4-(2-amino-pyridin-4- |
| | yloxy)-naphthalen-1-yl]urea |
| 266 | 1-(5-tert-butyl-2-methoxy-pyridin-3-yl)-3-[4-(2-methylamino- |
| | pyridin-4-yloxy]-naphthalen-1-yl}urea |
| 267 | 1-(5-tert-butyl-2-methoxy-pyridin-3-yl)-3-{4-[2-(1-phenyl- |
| | ethylamino)-pyridin-4-yloxy]-naphthalen-1-yl}urea |
| 268 | 1-(5-tert-butyl-2-methyl-phenyl)-3-[4-(2-aminopyridin-4-yl-oxy)- |
| | naphthalen-1-yl]urea |
| 269 | 1-(5-tert-butyl-2-morpholin-4-yl-phenyl)-3-[4-(2-morpholin-4-yl- |
| | ethoxy)-naphthalen-1-yl]urea |
| 270 | 1-(5-tert-butyl-2-methoxyphenyl)-3-[4-(2-methylpyridin-4-yl-oxy)- |
| | naphthalen-1-yl]urea |
| 271 | 1-(5-tert-butyl-2,3-dimethoxyphenyl)-3-[4-(2-methoxypyridin-4-yl- |
| | oxy)-naphthalen-1-yl]urea |
| 272 | 5-tert-butyl-2-methoxy-3-{3-[-(pyridin-4-yl-oxy)naphthalen-1- |
| | yl]ureido}benzamide |
| 273 | Morpholine-4-carboxylic acid (5-tert-butyl-2-methoxy-3-{3-[- |
| | (pyridin-4-yl-oxy)naphthalen-1-yl]ureido}phenyl)amide |
| 274 | N-(5-tert-butyl-2-methoxy-3-{3-[-(pyridin-4-yl-oxy)naphthalen-1- |
| | yl]}ureido}phenyl)acetamide |
| 275 | 3-(5-tert-butyl-2-methoxy-3-{3-[-(pyridin-4-yl-oxy)naphthalen-1- |
| | yl]ureido}phenyl)-1,1-dimethylurea |
| 276 | 1-(5-tert-butyl-2,3-dimethoxy-phenyl)-3-[4-(2-amino-pyridin-4- |
| | yloxy)-naphyhalen-1-yl]urea |
| 277 | 1-(5-tert-butyl-2,3-dimethoxy-phenyl)-3-[4-(2-methylamino-pyridin- |
| | 4-yloxy)-naphthalen-1-yl]urea |
| 278 | 1-(5-tert-butyl-2,3-dimethoxy-phenyl)-3-[4-(2-phenyl-ethylamino- |
| | pyridin-4-yloxy)-naphthalen-1-yl]urea |
| 279 | 1-(5-tert-butyl-2-methoxy-pyridin-3-yl)-3-[4-(2-amino-pyridin-4- |
| | yloxy)-naphthalen-1-yl]urea |
| 280 | 1-(5-tert-butyl-2-methoxy-pyridin-3-yl)-3-[4-(2-methylamino- |
| | pyridin-4-yloxy)-naphthalen-1-yl]urea |
| 281 | 1-(5-tert-butyl-2-methoxy-pyridin-3-yl)-3-{4-[2-(1-phenyl- |
| | ethylamino)-pyridin-4-yloxy)-naphthalen-1-yl]urea |
| 282 | 1-(5-tert-butyl-2-methyl-phenyl)-3-[4-(2-aminopyridin-4-yloxy)- |
| | naphthalen-1-yl]urea |
| 283 | 1-(5-tert-butyl-2-morpholin-4-yl-phenyl)-3-[4-(2-morpholin-4-yl- |
| | ethoxy)-naphthalen-1-yl]urea |

b) salts of the ureas within the above group,
c) prodrugs of the ureas within the above group and
d) isolated stereoisomers of the ureas within the above group.

19. A method as in claim 18 wherein the treated disease is characterized by abnormal angiogenesis or hyper-permeability processes.

20. A method as in claim 18 wherein the disease treated is selected from the group consisting of: tumor growth, retinopathy, ischemic retinal-vein occlusion, retinopathy of prematurity and age related macular degeneration; rheumatoid arthritis, psoriasis and a bullous disorder associated with subepidermal blister formation.

21. A method as in claim 18 wherein at least one additional angiogenesis inhibiting agent is administered with the one or more administered compounds defined in claim 12.

22. A method as in claim 21 wherein the disease treated is selected from the group consisting of: tumor growth, retinopathy, ischemic retinal-vein occlusion, retinopathy of prematurity and age related macular degeneration; rheumatoid arthritis, psoriasis, and a bullous disorder associated with subepidermal blister formation.

23. A method as in claim 21 wherein the one or more administered compounds and the one or more additional angiogenesis inhibiting agents are administered simultaneously in the same formulation.

24. A method as in claim 21 wherein the one or more administered compounds and the one or more additional angiogenesis inhibiting agents are administered separately in separate formulations.

25. A kit comprising a separate dose of the one or more compounds selected from the group consisting of:
a) ureas numbered 1-283 in claim 1
b) salts of the ureas numbered 1-283 in claim 1,
c) prodrugs of the ureas numbered 1-283 in claim 1 and
d) isolated stereoisomers of the ureas numbered 1-283 in claim 1 and a separate dose of the one or more additional angiogenesis inhibiting agents.

26. A method as in claim 18 wherein the compound administered is within a pharmaceutical composition which comprises a physiologically acceptable carrier.

27. A method for the treatment of a disease within a human or other mammal selected from the group consisting of:
rheumatoid arthritis, osteoarthritis, septic arthritis, tumor metastasis, periodontal disease, cornal ulceration, proteinuria, coronary thrombosis from atherosclerotic plaque, aneurismal aortic, birth control, dystrophobic epidermolysis bullosa, degenerative cartilage loss following traumatic joint injury, osteopenias mediated by MMP activity, tempero mandibular joint disease , demyelating disease of the nervous system and infectious diseases,
said method comprising administering one or more compounds selected from the group consisting of:
a) ureas selected from the group consisting of

| | |
|---|---|
| 1 | 1-[5-tert-butyl-2-(2-methylpyridin-5-yl)-2H-pyrazol-3-yl]-3-(4- |
| | chlorophenyl)urea |
| 2 | 1-(5-tert-butyl-2-p-tolyl-2H-pyrazol-3-yl)-3-(4-methoxynaphthalen- |
| | 1-yl)urea |
| 3 | 1-[5-tert-butyl-2-(3,4-dimethylphenyl)-2H-pyrazol-3-yl]-3-(4- |
| | fluorophenyl)urea |
| 4 | 1-[5-tert-butyl-2-(pyridin-3-yl)-2H-pyrazol-3-yl]-3-(4- |
| | cyanonaphthalen-1-yl)urea |
| 5 | 1-[5-tert-butyl-2-p-tolyl-2H-pyrazol-3-yl]-3-[4-(2-morpholin-4- |
| | ylethoxy)naphthalen-1-yl]-urea |
| 6 | 1-[5-tert-butyl-2-p-tolyl-2H-pyrazol-3-yl]-3-[4-(2-(cis-2,6- |
| | dimethylmorpholin-4-yl)ethoxy)naphthalen-1-yl]-urea |
| 7 | 1-[5-tert-butyl-2-p-tolyl-2H-pyrazol-3-yl]-3-[4-(2-(trans-2,6- |
| | dimethylmorpholin-4-yl)ethoxy)naphthalen-1-yl]-urea |
| 8 | 1-[5-tert-butyl-2-p-tolyl-2H-pyrazol-3-yl]-3-[4-(2-(2- |
| | (methoxymethyl)morpholin-4-yl)ethoxy)naphthalen-1-yl]-urea |
| 9 | 1-[5-tert-butyl-2-p-tolyl-2H-pyrazol-3-yl]-3-[4-(2- |
| | (morpholin-4-yl)-2-oxoethoxy)naphthalen-1-yl]-urea |
| 10 | 1-[5-tert-butyl-2-p-tolyl-2H-pyrazol-3-yl]-3-[4-(2-(morpholin-4-yl)- |
| | 2-methylethoxy)naphthalen-1-yl]-urea |
| 11 | 1-[5-tert-butyl-2-p-tolyl-2H-pyrazol-3-yl]-3-[4-(2-(morpholin-4-yl)- |
| | 1-methylethoxy)naphthalen-1-yl]-urea |
| 12 | 1-[5-tert-butyl-2-p-tolyl-2H-pyrazol-3-yl]-3-[4-(2-thiomorpholin-4- |
| | ylethoxy)naphthalen-1-yl]-urea |
| 13 | 1-[5-tert-butyl-2-p-tolyl-2H-pyrazol-3-yl]-3-[4-(2-(1- |
| | oxothiomorpholin-4-yl)ethoxy)naphthalen-1-yl]-urea |
| 14 | 1-[5-tert-butyl-2-p-tolyl-2H-pyrazol-3-yl]-3-[4-(2-morpholin-4-yl- |
| | ethoxy)-3-methylnaphthalen-1-yl]-urea |
| 15 | 1-[5-tert-butyl-2-p-tolyl-2H-pyrazol-3-yl]-3-[4-(2-(morpholin-4-yl- |
| | carbonyloxo)ethoxy)naphthalen-1-yl]-urea |
| 16 | 1-[5-tert-butyl-2-p-tolyl-2H-pyrazol-3-yl]-3-[4-(2-(tetrahydropyran- |
| | 4-yl)ethoxy)naphthalen-1-yl]-urea |
| 17 | 1-[5-tert-butyl-2-p-tolyl-2H-pyrazol-3-yl]-3-[4-(2-(1-oxo- |
| | tetrahydrothiophen-3-yl)ethoxy)naphthalen-1-yl]-urea |
| 18 | 1-[5-tert-butyl-2-p-tolyl-2H-pyrazol-3-yl]-3-[4-(3-morpholin-4-yl- |
| | propyl)naphthalen-1-yl]-urea |
| 19 | 1-[5-tert-butyl-2-p-tolyl-2H-pyrazol-3-yl]-3-[4-(3-morpholin-4-yl- |
| | methyl)naphthalen-1-yl]-urea |
| 20 | 1-[5-tert-butyl-2-p-tolyl-2H-pyrazol-3-yl]-3-[4-(2-pyridin-4-yl- |
| | ethyl)naphthalen-1-yl]-urea |
| 21 | 1-[5-tert-butyl-2-p-tolyl-2H-pyrazol-3-yl]-3-[4-(3-(morpholin-4- |
| | yl)propyn-1-yl)naphthalen-1-yl]-urea |
| 22 | 1-[5-tert-butyl-2-p-tolyl-2H-pyrazol-3-yl]-3-[4-(3-(tetrahydropyran- |
| | 2-yl-oxy)propyn-1-yl)naphthalen-1-yl]-urea |
| 23 | 1-[5-tert-butyl-2-p-tolyl-2H-pyrazol-3-yl]-3-[4-(3-(tetrahydropyran- |
| | 2-yl-oxy)butyn-1-yl)naphthalen-1-yl]-urea |
| 24 | 1-[5-tert-butyl-2-p-tolyl-2H-pyrazol-3-yl]-3-[4-(3-(piperidin-1- |
| | yl)propyn-1-yl)naphthalen-1-yl]-urea |
| 25 | 1-[5-tert-butyl-2-p-tolyl-2H-pyrazol-3-yl]-3-[4-(3-(2- |
| | methoxymethylmorpholin-4-yl)propyn-1-yl)naphthalen-1-yl]-urea |
| 26 | 1-[5-tert-butyl-2-p-tolyl-2H-pyrazol-3-yl]-3-[4-(pyridin-4-yl- |
| | methoxy)naphthalen-1-yl]-urea |
| 27 | 1-[5-tert-butyl-2-p-tolyl-2H-pyrazol-3-yl]-3-[4-(2-pyridin-4-yl- |
| | ethoxy)naphthalen-1-yl]-urea |
| 28 | 1-[5-tert-butyl-2-p-tolyl-2H-pyrazol-3-yl]-3-[4-(3-pyridin-4-yl- |
| | propoxy)naphthalen-1-yl]-urea |
| 29 | 1-[5-tert-butyl-2-p-tolyl-2H-pyrazol-3-yl]-3-[4-(2-imidazol-1-yl- |
| | ethoxy)naphthalen-1-yl]-urea |
| 30 | 1-[5-tert-butyl-2-p-tolyl-2H-pyrazol-3-yl]-3-[4-(2-(3,4- |
| | dimethoxyphenyl)-ethoxy)naphthalen-1-yl]-urea |
| 31 | 1-[5-tert-butyl-2-p-tolyl-2H-pyrazol-3-yl]-3-[4-(pyridin-4-yl- |
| | methylamino)naphthalen-1-yl]-urea |
| 32 | 1-[5-isopropyl-2-phenyl-2H-pyrazol-3-yl]-3-[4-(2-morpholin-4-yl- |
| | ethoxy)naphthalen-1-yl]-urea |
| 33 | 1-[5-cyclohexyl-2-phenyl-2H-pyrazol-3-yl]-3-[4-(2-morpholin-4-yl- |
| | ethoxy)naphthalen-1-yl]-urea |
| 34 | 1-[5-(2,2,2-trifluoroethyl)-2-phenyl-2H-pyrazol-3-yl]-3-[4-(2- |
| | morpholin-4-yl-ethoxy)naphthalen-1-yl]-urea |
| 35 | 1-[5-(1-methylcycloprop-1-yl)-2-phenyl-2H-pyrazol-3-yl]-3-[4-(2- |
| | morpholin-4-yl-ethoxy)naphthalen-1-yl]-urea |
| 36 | 1-[5-(1-methylcyclohex-1-yl)-2-phenyl-2H-pyrazol-3-yl]-3-[4-(2- |
| | morpholin-4-yl-ethoxy)naphthalen-1-yl]-urea |
| 37 | 1-[5-tert-butyl-2-methyl-2H-pyrazol-3-yl]-3-[4-(2-morpholin-4-yl- |
| | ethoxy)naphthalen-1-yl]-urea |
| 38 | 1-[5-tert-butyl-2-(4-chlorophenyl)-2H-pyrazol-3-yl]-3-[4-(2- |
| | morpholin-4-yl-ethoxy)naphthalen-1-yl]-urea |
| 39 | 1-[5-tert-butyl-2-butyl-2H-pyrazol-3-yl]-3-[4-(2-morpholin-4-yl- |
| | ethoxy)naphthalen-1-yl]-urea |
| 40 | 1-[5-tert-butyl-2-(4-methyl-3-carbamylphenyl)-2H-pyrazol-3-yl]-3- |
| | [4-(2-morpholin-4-yl-ethoxy)naphthalen-1-yl]-urea |
| 41 | 1-[5-tert-butyl-2-(4-methyl-3-(morpholin-4-yl)methylphenyl)-2H- |
| | pyrazol-3-yl]-3-[4-(2-morpholin-4-yl-ethoxy)naphthalen-1-yl]-urea |
| 42 | 1-[5-tert-butyl-2-(4-methyl-3-dimethylaminomethylphenyl)-2H- |
| | pyrazol-3-yl]-3-[4-(2-morpholin-4-yl-ethoxy)naphthalen-1-yl]-urea |
| 43 | 1-[5-tert-butyl-2-(3-dimethylaminomethylphenyl)-2H-pyrazol- |
| | 3-yl]-3-[4-(2-morpholin-4-yl-ethoxy)naphthalen-1-yl]-urea |
| 44 | 1-[5-tert-butyl-2-(2-chloropyridin-5-yl)-2H-pyrazol-3-yl]-3-[4-(2- |
| | morpholin-4-yl-ethoxy)naphthalen-1-yl]-urea |
| 45 | 1-[5-tert-butyl-2-(2-methylpyridin-5-yl)-2H-pyrazol-3-yl]-3-[4-(2- |
| | morpholin-4-yl-ethoxy)naphthalen-1-yl]-urea |
| 46 | 1-[5-tert-butyl-2-(2-methoxypyridin-5-yl)-2H-pyrazol-3-yl]-3-[4-(2- |
| | morpholin-4-yl-ethoxy)naphthalen-1-yl]-urea |
| 47 | 1-[5-tert-butyl-2-(pyridin-3-yl)-2H-pyrazol-3-yl]-3-[4- |
| | (2-morpholin-4-yl-ethoxy)naphthalen-1-yl]-urea |
| 48 | 1-[5-tert-butyl-2-(2-methylpyridin-5-yl)-2H-pyrazol-3-yl]-3-[4-(2- |
| | pyridin-4-yl-ethoxy)naphthalen-1-yl]-urea |
| 49 | 1-[5-tert-butyl-2-(2-methylpyridin-5-yl)-2H-pyrazol-3-yl]-3-[4-(2- |
| | (trans-2,6-dimethylmorpholin-4-yl)ethoxy)naphthalen-1-yl]-urea |
| 50 | 1-[5-tert-butyl-2-(2-methylpyridin-5-yl)-2H-pyrazol-3-yl]-3-[4-(3- |
| | morpholin-4-yl-propyn-1-yl)naphthalen-1-yl]-urea |
| 51 | 1-[5-(2-hydroxy-1,1-dimethyl-ethyl)-2-p-tolyl-2H-pyrazol-3-yl]-3-[ |
| | 4-(2-morpholin-4-yl-ethoxy)naphthalen-1-yl]-urea |
| 52 | 1-[5-tert-butyl-2-(3-hydroxy-4-methyl-phenyl)-2H-pyrazol-3-yl]-3-[ |
| | 4-(2-morpholin-4-yl-ethoxy)naphthalen-1-yl]-urea |
| 53 | 1-[5-tert-butyl-2-(4-hydroxymethyl-phenyl)-2H-pyrazol-3-yl]-3-[4- |
| | (2-morpholin-4-yl-ethoxy)naphthalen-1-yl]-urea |
| 54 | 1-[5-tert-butyl-2-p-tolyl-2H-pyrazol-3-yl]-3-{4-[2-(3-oxo- |
| | morpholin-4-yl)-ethoxy]naphthalen-1-yl}-urea |
| 55 | 1-[5-tert-butyl-2-p-tolyl-2H-pyrazol-3-yl]-3-{4-[2-(4-hydroxy- |
| | morpholin-4-yl)-ethoxy]naphthalen-1-yl}-urea |
| 56 | 1-[5-(2-hydroxy-1,1-dimethyl-ethyl)-2-(6-methyl-pyridin-3-yl)-2H- |
| | pyrazol-3-yl]-3-[4-(2-morpholin-4-yl-ethoxy)naphthalen-1-yl]-urea |
| 57 | 1-[5-tert-butyl-2-(1-hydroxy-6-methyl-pyridin-3-yl)-2H-pyrazol-3- |
| | yl]-3-[4-(2-morpholin-4-yl-ethoxy)naphthalen-1-yl]-urea |
| 58 | 1-[5-tert-butyl-2-(6-methyl-pyridin-3-yl)-2H-pyrazol-3-yl)-3-{4-[2- |
| | (4-hydroxy-morpholin-4-yl)-ethoxy]naphthalen-1-yl}-urea |
| 59 | 1-[5-(2-hydroxy-1,1-dimethyl-ethyl)-2-(6-methyl-pyridin-3-yl)-2H- |
| | pyrazol-3-yl]-3-[4-(2-pyridin-4-yl-ethoxy)naphthalen-1-yl]-urea |
| 60 | 1-[5-tert-butyl-2-(6-methyl-pyridin-3-yl)-2H-pyrazol-3-yl]-3-[4-(2- |
| | hydroxy-2-pyridin-4-yl-ethoxy)naphthalen-1-yl]-urea |
| 61 | 1-[5-tert-butyl-2-(6-methyl-pyridin-3-yl)-2H-pyrazol-3-yl]-3-{4-[2- |
| | (1-hydroxy-2-pyridin-4-yl)-ethoxy]naphthalen-1-yl}-urea |
| 62 | 1-[5-(2-hydroxy-1,1-dimethyl-ethyl)-2-p-tolyl-2H-pyrazol-3-yl]-3-{ |
| | 4-[2-(1-oxo-thiomorpholin-4-yl)-ethoxy]naphthalen-1-yl}-urea |
| 63 | 1-[5-tert-butyl-2-(4-hydroxymethyl-phenyl)-2H-pyrazol-3-yl]-3-{4- |
| | [2-(1-oxo-thiomorpholin-4-yl)-ethoxy]naphthalen-1-yl}-urea |
| 64 | 1-[5-tert-butyl-2-p-tolyl-2H-pyrazol-3-yl]-3-{4-[2-(1,3-dioxo- |
| | thiomorpholin-4-yl)-ethoxy]naphthalen-1-yl}-urea |
| 65 | 1-[5-(2-hydroxy-1,1-dimethyl-ethyl)-2-methyl-2H-pyrazol-3-yl]-3-[ |
| | 4-(2-morpholin-4-yl-ethoxy)-naphthalen-1-yl]-urea |
| 66 | 1-[5-tert-butyl-2-methyl-2H-pyrazol-3-yl]-3-{4-[2-(4-hydroxy- |
| | morpholin-4-yl)-ethoxy]-naphthalen-1-yl}-urea |
| 67 | 1-[5-tert-butyl-2-p-tolyl-2H-pyrazolyl-3-yl]-3-[4-(4-morpholin- |
| | 4-yl-methyl)phenyl)naphthalen-1-yl]urea |
| 68 | 1-[5-tert-butyl-2-p-tolyl-2H-pyrazolyl-3-yl]-3-[4-(4-(2-(morpholin- |
| | 4-yl)ethyl)phenyl)naphthalen-1-yl]urea |
| 69 | 1-[5-tert-butyl-2-p-tolyl-2H-pyrazolyl-3-yl]-3-[4-(3-(morpholin- |
| | 4-yl-methyl)phenyl)naphthalen-1-yl]urea |
| 70 | 1-[5-tert-butyl-2-p-tolyl-2H-pyrazolyl-3-yl]-3-[4-(6-(morpholin-4- |
| | ylmethyl-pyridin-3-yl)naphthalen-1-yl]urea |
| 71 | 1-[5-tert-butyl-2-p-tolyl-2H-pyrazolyl-3-yl]-3-[4-(5-(morpholin-4- |
| | ylmethyl-pyridin-2-yl)naphthalen-1-yl]urea |
| 72 | 1-[5-tert-butyl-2-p-tolyl-2H-pyrazolyl-3-yl]-3-[4-(5-(morpholin-4- |
| | ylmethyl-fur-2-yl)naphthalen-1-yl]urea |
| 73 | 1-[5-tert-butyl-2-(6-methyl-pyridin-3-yl)-2H-pyrazolyl-3-yl]-3-[4- |
| | (6-(morpholin-4-ylmethyl-pyridin-3-yl)naphthalen-1-yl]urea |
| 74 | 1-[5-tert-butyl-2-methyl-2H-pyrazolyl-3-yl]-3-[4-(6-morpholin-4- |
| | ylmethyl-pyridin-3-yl)naphthalen-1-yl]urea |
| 75 | 1-[5-tert-butyl-2-p-tolyl-2H-pyrazolyl-3-yl]-3-[4-(5-(morpholin-4- |
| | yl-methyl)pyridin-2-yl)naphthalen-1-yl]urea |
| 76 | 1-[5-tert-butyl-2-p-tolyl-2H-pyrazolyl-3-yl]-3-[4-(6-(morpholin-4- |
| | yl-methyl)pyridin-3-yl)naphthalen-1-yl]urea |
| 77 | 1-[5-tert-butyl-2-(6-methyl-pyridin-3-yl)-2H-pyrazolyl-3-yl]-3-[4- |
| | (3-(2-(pyridin-2-yl)ethylamino)cyclohexenyl)-naphthalen-1-yl]urea |
| 78 | 1-[5-tert-butyl-2-p-tolyl-2H-pyrazolyl-3-yl]-3-[4-(4-(pyridin-3-yl- |
| | methylaminomethyl)phenyl)naphthalen-1-yl]urea |
| 79 | 1-[5-tert-butyl-2-(6-methyl-pyridin-3-yl)-2H-pyrazolyl-3-yl]-3-[4- |
| | (4-(morpholin-4-yl-methyl)phenyl)naphthalen-1-yl]urea |
| 80 | 1-[5-tert-butyl-2-(6-methyl-pyridin-3-yl)-2H-pyrazolyl-3-yl]-3-[4- |
| | (6-(4-(hydroxybutylamino)pyridin-3-yl)-naphthalen-1-yl]urea |
| 81 | 1-[5-tert-butyl-2-(4-methyl-3-carbamylphenyl)-2H-pyrazolyl-3-yl]- |
| | 3-[4-(6-(morpholin-4-yl-methyl)pyridin-3-yl)naphthalen-1-yl]urea |
| 82 | 1-[5-tert-butyl-2-(6-methyl-pyridin-3-yl)-2H-pyrazolyl-3-yl]-3-[4- |
| | (4-(3-hydroxypiuperidin-1-yl-methyl)phenyl)naphthalen-1-yl]urea |
| 83 | 1-[5-tert-butyl-2-(6-methyl-pyridin-3-yl)-2H-pyrazolyl-3-yl]-3-[4- |
| | (4-(4-hydroxymorpholin-4-yl-methyl)phenyl)naphthalen-1-yl]urea |
| 84 | 1-[5-tert-butyl-2-(6-methyl-pyridin-3-yl)-2H-pyrazolyl-3-yl]-3-[4- |
| | (3-(morpholin-4-yl-methyl)cyclohexenyl)naphthalen-1-yl]urea |
| 85 | 1-[5-tert-butyl-2-(6-methyl-pyridin-3-yl)-2H-pyrazolyl-3-yl]-3-[4- |
| | (4-(tetrahydrofuran-3-yl-methyl)-3-hydroxyphenyl)naphthalen-1- |
| | yl]urea |
| 86 | 1-[5-tert-butyl-2-(6-methyl-pyridin-3-yl)-2H-pyrazolyl-3-yl]-3-[4- |
| | (4-(N,N-di-(2-methoxyethyl)aminomethyl)phenyl)naphthalen-1- |
| | yl]urea |
| 87 | 1-[5-tert-butyl-2-(6-methyl-pyridin-3-yl)-2H-pyrazolyl-3-yl]-3-[4- |
| | (6-(cyanopropoxy)pyridin-3-yl)naphthalen-1-yl]urea |
| 88 | 1-[5-tert-butyl-2-(6-methyl-pyridin-3-yl)-2H-pyrazolyl-3-yl]-3-[4- |
| | (4-morpholin-4-yl-methyl-piperidinyl)naphthalen-1-yl]urea |
| 89 | 1-[5-tert-butyl-2-(6-methyl-pyridin-3-yl)-2H-pyrazolyl-3-yl]-3-[4- |
| | (4-(N,N-di-(2-cyanoethyl)aminomethyl)phenyl)naphthalen-1- |
| | yl]urea |
| 90 | 1-[5-tert-butyl-2-(6-methyl-pyridin-3-yl)-2H-pyrazolyl-3-yl]-3-[4- |
| | (4-(furan-2-yl-methyl)-3-hydroxyphenyl)naphthalen-1-yl]urea |
| 91 | 1-[5-tert-butyl-2-(6-methyl-pyridin-3-yl)-2H-pyrazolyl-3-yl]-3-[4- |
| | (4-(thiomorpholin-4-yl-methyl)phenyl)naphthalen-1-yl]urea |
| 92 | 1-[5-tert-butyl-2-(6-methyl-pyridin-3-yl)-2H-pyrazolyl-3-yl]-3-[4- |
| | (4-(3-carboxamidopiperidin-1-yl-methyl)phenyl)naphthalen-1- |
| | yl]urea |
| 93 | 1-[5-tert-butyl-2-(6-methyl-pyridin-3-yl)-2H-pyrazolyl-3-yl]-3-[4- |
| | (4-(2-methyl-3-oxo-piperzin-1-yl-methyl)phenyl)naphthalen-1- |
| | yl]urea |
| 94 | 1-[5-tert-butyl-2-(2-methylpyrimidin-5-yl)-2H-pyrazolyl-3-yl]-3-[4- |
| | (6-(morpholin-4-yl-methyl)pyridin-3-yl)naphthalen-1-yl]urea |
| 95 | 1-[5-tert-butyl-2-(6-methyl-pyridin-3-yl)-2H-pyrazolyl-3-yl]-3-[4- |
| | (6-(4-hydroxybutyloxy)pyridin-3-yl)naphthalen-1-yl]urea |
| 96 | 1-[3-tert-butyl-1′H-[1,4′]bipyrazol-5-yl]-3-[4-(6-(morpholin-4-yl- |
| | methyl)pyridin-3-yl)naphthalen-1-yl]urea |
| 97 | 1-[5-tert-butyl-2--2H-pyrazolyl-3-yl]-3-[4-(6-(tetrahydrothiopyran- |
| | 4-yl-amino)pyridin-3-yl)naphthalen-1-yl]urea |
| 98 | 1-[5-tert-butyl-2-(2-cyanoethyl)-2H-pyrazolyl-3-yl]-3-[4-(6- |
| | (morpholin-4-yl-methyl)pyridin-3-yl)naphthalen-1-yl]urea |
| 99 | 1-[5-tert-butyl-2-(6-methyl-pyridin-3-yl)-2H-pyrazolyl-3-yl]-3-[4- |
| | (6-(2,6-dimethylmorpholin-4-yl-methyl)pyridin-3-yl)naphthalen-1- |
| | yl]urea |
| 100 | 1-[5-tert-butyl-2-(2-methoxypyridin-5-yl)-2H-pyrazolyl-3-yl]-3-[4- |
| | (6-(morpholin-4-yl-methyl)pyridin-3-yl)naphthalen-1-yl]urea |
| 101 | 1-[5-tert-butyl-2-(2-aminopyridin-5-yl)-2H-pyrazolyl-3-yl]-3-[4- |
| | (6-(morpholin-4-yl-methyl)pyridin-3-yl)naphthalen-1-yl]urea |
| 102 | 1-[5-tert-butyl-2-(6-methyl-pyridin-3-yl)-2H-pyrazolyl-3-yl]-3-[4- |
| | (6-morpholin-4-yl-4-carbonyl)pyridin-3-yl)naphthalen-1-yl]urea |
| 103 | 1-[5-tert-butyl-2-(6-methyl-pyridin-3-yl)-2H-pyrazolyl-3-yl]-3-[4- |
| | (6-(2-oxa-5-aza-bicyclo[2.2.1]hept-5-yl-methyl)pyridin-3-yl) |
| | naphthalen-1-yl]urea |
| 104 | 1-[5-tert-butyl-2-(6-methyl-pyridin-3-yl)-2H-pyrazolyl-3-yl]-3-[4- |
| | (4-(N-(2-cyanoethyl)-N-(pyridin-3- |
| | ylmethyl)aminomethyl)phenyl)naphthalen-1-yl]urea |
| 105 | 1-[5-tert-butyl-2-(6-methyl-pyridin-3-yl)-2H-pyrazolyl-3-yl]-3-[4- |
| | (4-(N-(2-cyanoethyl)-N-(tetrahydrofuran-2-yl- |
| | methyl)aminomethyl)phenyl)naphthalen-1-yl]urea |
| 106 | 1-[5-tert-butyl-2-(6-methyl-pyridin-3-yl)-2H-pyrazolyl-3-yl]-3-[4- |
| | (6-(morpholin-4-yl-methyl)-4-methoxypyridin-3-yl)naphthalen-1- |
| | yl]urea |
| 107 | 1-[5-tert-butyl-2-(6-methyl-pyridin-3-yl)-2H-pyrazolyl-3-yl]-3-[4- |
| | (6-(1-morpholin-4-yl-propyl)pyridin-3-yl)naphthalen-1-yl]urea |
| 108 | 1-[3-tert-butyl-1′-methyl-1′H-[1,4′]bipyrazol-5-yl]-3-[4- |
| | (6-(morpholin-4-yl-methyl)pyridin-3-yl)naphthalen-1-yl]urea |
| 109 | 1-[5-tert-butyl-2-(6-methyl-pyridin-3-yl)-2H-pyrazolyl-3-yl]-3-[4- |
| | (6-(1-oxo-tetrahydrothiopyran-4-yl-amino)pyridin-3-yl)naphthalen- |
| | 1-yl]urea |
| 110 | 1-[5-tert-butyl-2-(6-methyl-pyridin-3-yl)-2H-pyrazolyl-3-yl]-3-[4- |
| | (6-(tetrahydropyran-4-yl-amino)pyridin-3-yl)naphthalen-1-yl]urea |
| 111 | 1-[5-tert-butyl-2-(6-methyl-pyridin-3-yl)-2H-pyrazolyl-3-yl]-3-[4- |
| | (5-(tetrahydrothiopyran-4-yl-aminbo)pyrazin-2-yl)naphthalen-1- |
| | yl]urea |
| 112 | 1-[5-tert-butyl-2-(6-methyl-pyridin-3-yl)-2H-pyrazolyl-3-yl]-3-[4- |
| | (6-(metrhylcarbonylamino)pyridin-3-yl)naphthalen-1-yl]urea |
| 113 | 1-[5-tert-butyl-1′-(3-methylsulfanylpropyl)-1′H-[1,4′]bipyrazol- |
| | 5-yl]-3-[4-(6-(morpholin-4-yl-methyl)pyridin-3-yl)naphthalen-1- |
| | yl]urea |
| 114 | 1-[5-tert-butyl-2-(2-methylpyrimidin-5-yl)-2H-pyrazolyl-3-yl]-3-[4- |
| | (6-(1-oxo-thiomorpholin-4-yl-methyl)pyridin-3-yl)naphthalen-1- |
| | yl]urea |
| 115 | 1-[5-tert-butyl-2-(2-methylpyrimidin-5-yl)-2H-pyrazolyl-3-yl]-3-[4- |
| | (6-(tetrahydropyran-4-ylamino)pyridin-3-yl)naphthalen-1-yl]urea |
| 116 | 1-[5-tert-butyl-2-(2-methylthiopyrimidin-5-yl)-2H-pyrazolyl-3-yl]- |
| | 3-[4-(6-(morpholin-4-yl-methyl)pyridin-3-yl)naphthalen-1-yl]urea |
| 117 | 1-[5-tert-butyl-2-(2-aminopyrimidin-5-yl)-2H-pyrazolyl-3-yl]-3-[4- |
| | (6-(morpholin-4-yl-methyl)pyridin-3-yl)naphthalen-1-yl]urea |
| 118 | 1-[3-tert-butyl-1′-methyl-1′H-[1,4;]bipyrazol-5-yl]-3-[4-(6- |
| | (morpholin-4-yl-methyl)phenyl)naphthalen-1-yl]urea |
| 119 | 1-[5-tert-butyl-2-p-tolyl-2H-pyrazolyl-3-yl]-3-[4-(6-(1-oxo- |
| | tetrahydrothiopyran-4-yl-amino)pyridin-3-yl)naphthalen-1-yl]urea |
| 120 | 1-[5-tert-butyl-2-p-tolyl-2H-pyrazolyl-3-yl]-3-[4-(6-(thiomorpholin- |
| | 4-yl-methyl)pyridin-3-yl)naphthalen-1-yl]urea |
| 121 | 1-[5-tert-butyl-2-p-tolyl-2H-pyrazolyl-3-yl]-3-[4-(2-(morpholin-4- |
| | yl-carbonyl)pyrimidin-5-yl)naphthalen-1-yl]urea |
| 122 | 1-[5-tert-butyl-2-p-tolyl-2H-pyrazolyl-3-yl]-3-[4-(4-(morpholin-4- |
| | yl-methyl)pyrimidin-5-yl)naphthalen-1-yl]urea |
| 123 | 1-[5-tert-butyl-2-p-tolyl-2H-pyrazolyl-3-yl]-3-[4-(6-(1-oxo- |
| | thiomorpholin-4-yl-methyl)pyridin-3-yl)naphthalen-1-yl]urea |
| 124 | 1-[5-tert-butyl-2-(2-methylpyrimidin-5-yl)-2H-pyrazolyl-3-yl]-3-[4- |
| | (2-(morpholin-4-yl-methyl)pyrimidin-5-yl)naphthalen-1-yl]urea |
| 125 | 1-[2-tert-butyl-5-methyl-pyridin-4-yl]-3-[4-(6-(morpholin-4-yl- |
| | methyl-pyridin-3-yl)naphthalen-1-yl]urea |
| 126 | 1-[3-tert-butyl-phenyl]-3-[4-(6-(morpholin-4-yl-methyl)pyridin-3- |
| | yl)naphthalen-1-yl]urea |
| 127 | 1-[4-methyl-biphenyl-3-yl]-3-[4-(6-(morpholin-4-yl-methyl- |
| | pyridin-3-yl)naphthalen-1-yl]urea |
| 128 | 1-[4-tert-butyl-biphenyl-2-yl]-3-[4-(6-morpholin-4-yl-methyl- |
| | pyridin-3-yl)naphthalen-1-yl]urea |
| 129 | 1-[5-isopropyl-2-methyl-phenyl]-3-[4-(6-morpholin-4-yl-methyl- |
| | pyridin-3-yl)naphthalen-1-yl]urea |
| 130 | 1-[5-sec-butyl-2-methoxy-phenyl]-3-[4-(6-(morpholin-4-yl-methyl- |
| | pyridin-3-yl)naphthalen-1-yl]urea |
| 131 | 1-[5-tert-butyl-2-methoxymethyl-phenyl]-3-[4-(6-(morpholin-4-yl- |
| | methyl-pyridin-3-yl)naphthalen-1-yl]urea |
| 132 | 1-[5-tert-butyl-2-methoxy-phenyl]-3-[4-(6-(morpholin-4-yl-methyl- |
| | pyridin-3-yl)naphthalen-1-yl]urea |
| 133 | 1-[5-tert-butyl-2-mewthyl-phenyl]-3-{6-[(3-(methoxy-propyl)- |
| | methyl-amino]-pyridin-3-yl}naphthalen-1-yl]urea |
| 134 | 1-[5-tert-butyl-2-methyl-phenyl]-3-[4-(6-morpholin-4-yl-methyl- |
| | pyridin-3-yl)naphthalen-1-yl]urea |
| 135 | 1-[5-tert-butyl-2-methyl-pyridin-3-yl]-3-[4-(6-morpholin-4-yl- |
| | methyl-pyridin-3-yl)naphthalen-1-yl]urea |
| 136 | 1-[5-(1,1-dimethylpropyl)-2-methoxy-phenyl]-3-[4-(6-morpholin-4- |
| | yl-methyl-pyridin-3-yl)naphthalen-1-yl]urea |
| 137 | 1-[5-tert-butyl-2-(1H-pyrazol-4-yl)-phenyl]-3-[4-(6-morpholin-4-yl- |
| | methyl-pyridin-3-yl)naphthalen-1-yl]urea |
| 138 | 1-[5-tert-butyl-2-(2-methyl-pyrimidin-5-yl)-phenyl]-3-[4-(6- |
| | morpholin-4-yl-methyl-pyridin-3-yl)naphthalen-1-yl]urea |
| 139 | 1-[5-tert-butyl-2-(3-hydroxy-propyl)-phenyl]-3-[4-(6-morpholin-4- |
| | yl-methyl-pyridin-3-yl)naphthalen-1-yl]urea |
| 140 | 1-[5-tert-butyl-2-(morpholine-4-carbonyl)-phenyl]-3-[4-(6- |
| | morpholin-4-yl-methyl-pyridin-3-yl)naphthalen-1-yl]urea |
| 141 | 1-[5-tert-butyl-2-methoxy-3-{3-[4-(6-morpholin-4-ylmethyl- |
| | pyridin-3-yl)-naphthalen-1-yl]-ureido}-phenyl)-acetamide |
| 142 | 1-[3-methyl-naphthalen-2-yl]-3-[4-(6-morpholin-4-yl-methyl- |
| | pyridin-3-yl)naphthalen-1-yl]urea |
| 143 | 1-[5-tert-butyl-2-methoxy-3-{3-[4-(6-morpholin-4-ylmethyl- |
| | pyridin-3-yl)-naphthalen-1-yl]-urido}-phenyl)-acetamide |
| 144 | 1-[5-tert-butyl-3-(2,3-dihydroxy-propyl)-2-hydroxy-phenyl]-3-[4- |
| | (6-morpholin-4-yl-methyl-pyridin-3-yl)naphthalen-1-yl]urea |
| 145 | 1-[2,3-dimethyl-1H-indol-5-yl]-3-[4-(6-morpholin-4-yl-methyl- |
| | pyridin-3-yl)naphthalen-1-yl]urea |
| 146 | 1-{5-tert-butyl-2-methyl-3-[3-(tetrahydro-pyran-2-yloxy)-prop-1- |
| | ynyl]-phenyl}-3-[4-(6-morpholin-4-yl-methyl-pyridin-3- |
| | yl)naphthalen-1-yl]urea |
| 147 | 1-[2-methoxy-5-trifluoromethyl-phenyl]-3-[4-(6-morpholin-4-yl- |
| | methyl-pyrimidin-3-yl)naphthalen-1-yl]urea |
| 148 | 1-[5-(2,2-dimethyl-propionyl)-2-methyl-phenyl]-3-[4-(6-morpholin- |
| | 4-yl-methyl-pyridin-3-yl)naphthalen-1-yl]urea |
| 149 | 1-[5-tert-butyl-3-(3-hydroxy-prop-1-ynyl)-2-methyl-phenyl]-3-[4- |
| | (6-morpholin-4-yl-methyl-pyridin-3-yl)naphthalen-1-yl]urea |
| 150 | 1-[5-tert-butyl-2-(3-hydroxy-propyn-1-ynyl)-phenyl]-3-[4-(6- |
| | morpholin-4-yl-methyl-pyridin-3-yl)naphthalen-1-yl]urea |
| 151 | 1-[5-tert-butyl-3-(2,2-dimethyl-[1,3]dioxolan-4-ylmethyl)-2- |
| | methoxy-phenyl]-3-[4-(6-morpholin-4-yl-methyl-pyridin-3- |
| | yl)naphthalen-1-yl]urea |
| 152 | 1-[5-tert-butyl-3-(2,3-dihydroxy-propyl)-2-methoxy-phenyl]-3-[4- |
| | (6-morpholin-4-yl-methyl-pyridin-3-yl)naphthalen-1-yl]urea |
| 153 | 1-[5-tert-butyoxy-2-methoxy-phenyl]-3-[4-(6-morpholin-4-yl- |
| | methyl-pyridin-3-yl)naphthalen-1-yl]urea |
| 154 | 1-[5-(1-cyano-cyclopropyl)-2-methoxy-phenyl]-3-[4-(6-morpholin- |
| | 4-yl-methyl-pyridin-3-yl)naphthalen-1-yl]urea |
| 155 | 1-[5-tert-butyl-3-(2-diethylamino-ethyl)-2-methoxy-phenyl]-3-[4- |
| | (6-morpholin-4-yl-methyl-pyridin-3-yl)naphthalen-1-yl]urea |
| 156 | 1-[5-tert-butyl-2-methoxy-phenyl]-3-[4-(6-[1,3]dioxolan-2-yl- |
| | pyridin-3-yl)naphthalen-1-yl]urea |
| 157 | 1-[5-tert-butyl-2-pyrrolidin-1-yl-phenyl]-3-[4-(6-morpholin-4-yl- |
| | methyl-pyridin-3-yl)naphthalen-1-yl]urea |
| 158 | 1-[5-tert-butyl-2-dimethylamino-phenyl]-3-[4-(6-morpholin-4-yl- |
| | methyl-pyridin-3-yl)naphthalen-1-yl]urea |
| 159 | 1-[5-tert-butyl-2-propoxy-phenyl]-3-[4-(6-morpholin-4-yl-methyl- |
| | pyridin-3-yl)naphthalen-1-yl]urea |
| 160 | 1-[5-tert-butyl-2-methoxy-phenyl]-3-[4-(6-hydroxymethyl-pyridin- |
| | 3-yl)naphthalen-1-yl]urea |
| 161 | 1-[5-tert-butyl-2-methoxy-phenyl]-3-{4-[6-(2,6-dimethyl- |
| | morpholin-4-yl-methyl)pyridin-3-yl]naphthalen-1-yl}urea |
| 162 | 1-[5-cyclohexyl-2-methoxy-phenyl]-3-[4-(6-morpholin-4-yl- |
| | methyl-pyridin-3-yl)naphthalen-l-yl]urea |
| 163 | 1-[2,4-dimethoxy-5-trifluoromethyl-phenyl]-3-[4-(6-morpholin-4- |
| | yl-methyl-pyridin-3-yl)naphthalen-1-yl]urea |
| 164 | 1-[5-tert-butyl-2-methoxy-3-nitro-phenyl]-3-[4-(6-morpholin-4-yl- |
| | methyl-pyridin-3-yl)naphthalen-1-yl]urea |
| 165 | 1-[3-amino-5-tert-butyl-2-methoxy-phenyl]-3-[4-(6-methyl-pyridin- |
| | 3-yl)naphthalen-1-yl]urea |
| 166 | N-acetyl-N-(5-tert-butyl-2-methoxy-3-{3-[4-(6-morpholin-4- |
| | ylmethyl-pyridin-3-yl)-naphthalen-1-yl]-ureido}-phenyl)-acetamide |
| 167 | 1-[6-tert-butyl-4-methyl-3-oxo-3,4-dihydro-2H-benzo[1,4]oxazin- |
| | 8-yl]-3-[4-(6-morpholin-4-ylmethyl-pyridin-3-yl)naphthalen-1- |
| | yl]urea |
| 168 | 1-[5-tert-butyl-2-ethoxy-phenyl]-3-[4-(6-morpholin-4-yl-methyl- |
| | pyridin-3-yl)naphthalen-1-yl]urea |
| 169 | 1-[5-tert-butyl-2-isopropoxy-phenyl]-3-[4-(6-morpholin-4-yl- |
| | methyl-pyridin-3-yl)naphthalen-1-yl]urea |
| 170 | 1-[5-tert-butyl-2-imidazol-1-yl-phenyl]-3-[4-(6-morpholin-4-yl- |
| | methyl-pyridin-3-yl)naphthalen-1-yl]urea |
| 171 | 1-[5-tert-butyl-3-ethylamino-2-methoxy-phenyl]-3-[4- |
| | (6-morpholin-4-yl-methyl-pyridin-3-yl)naphthalen-1-yl]urea |
| 172 | 1-[5-tert-butyl-2-methoxy-3-{3-[4-(6-morpholin-4-ylmethyl- |
| | pyridin-3-yl)-naphthalen-1-yl]-ureido}-phenyl)- |
| | bis(methanesulfon)amide |
| 173 | 1-[5-tert-butyl-2-(1-methyl-1H-pyrazol-4-yl)-phenyl]-3-[4-(6- |
| | morpholin-4-yl-methyl-pyridin-3-yl)naphthalen-1-yl]urea |
| 174 | 1-[2-methanesulfinyl-5-trifluoromethyl-phenyl]-3-[4-(6-morpholin- |
| | 4-yl-methyl-pyridin-3-yl)naphthalen-1-yl]urea |
| 175 | 1-[4-(6-{[bis(2-methoxy-ethyl)-amino]-methyl}-pyridin-3-yl- |
| | naphthalen-1-yl]-3-[5-tert-2-methoxy-phenyl]urea |
| 176 | N-[1-(5-{4-[3-(5-tert-butyl-2-methoxy-phenyl)-ureido]-naphthalen- |
| | 1-yl}-pyridin-2-ylmethyl)-pyrrolidin-3-yl]-acetamide |
| 177 | 1-[1-acetyl-3,3-dimethyl-2,3-dihydro-1H-indol-5-yl]-3-[4-(6- |
| | morpholin-4-yl-methyl-pyridin-3-yl)naphthalen-1-yl]urea |
| 178 | 1-(5-tert-butyl-2-methoxy-3-{3-[4-(6-morpholin-4-ylmethyl- |
| | pyridin-3-yl)-naphthalen-1-yl]-ureido}-phenyl)-propionamide |
| 179 | 1-[5-tert-butyl-2-methyl-benzooxazol-7-yl]-3-[4-(6-morpholin-4-yl- |
| | methyl-pyridin-3-yl)naphthalen-1-yl]urea |
| 180 | 1-[3-trifluoromethanesulfonyl-phenyl]-3-[4-(6-morpholin-4-yl- |
| | methyl-pyridin-3-yl)naphthalen-1-yl]urea |
| 181 | N-(5-tert-butyl-2-methoxy-3-{3-{4-(6-morpholin-4-ylmethyl- |
| | pyridin-3-yl)-naphthalen-1-yl]-ureido}-phenyl)-isobutyramide |
| 182 | 2-(4-tert-butyl-2-{3-[4-(6-morpholin-4-ylmethyl-pyridin-3-yl)- |
| | naphthalen-1-yl]-ureido}-phenoxy)-acetamide |
| 183 | 1-[5-tert-butyl-2-oxo-2,3-dihydro-benzooxazol-7-yl]-3-[4-(6- |
| | morpholin-4-yl-methyl-pyridin-3-yl)naphthalen-1-yl]urea |
| 184 | 1-[5-tert-butyl-3-cyano-2-methoxy-phenyl]-3-[4-(6-morpholin-4-yl- |
| | methyl-pyridin-3-yl)naphthalen-1-yl]urea |
| 185 | 1-[5-tert-butyl-benzooxazol-7-yl]-3-[4-(6-morpholin-4-yl-methyl- |
| | pyridin-3-yl)naphthalen-1-yl]urea |
| 186 | 5-tert-butyl-2-methoxy-{3-[4-(6-morpholin-4-ylmethyl-pyridin-3- |
| | yl)-naphthalen-1-yl]-ureido}-phenyl)-benzenesulfonamide |
| 187 | Ethanesulfonic acid (5-tert-butyl-2-methoxy-3-{3-[4-(6-morpholin- |
| | 4-ylmethyl-pyridin-3-yl)-naphthalen-1-yl]-ure3ido}-phenyl)-amide |
| 188 | 1-[5-tert-butyl-2-methoxy-phenyl]-3-[4-(2-morpholin-4-yl-methyl- |
| | pyrimidin-5-yl)naphthalen-1-yl]urea |
| 189 | 1-[5-tert-butyl-2-methylsulfanyl-phenyl]-3-[4-(6-morpholin-4-yl- |
| | methylpyridin-3-yl)naphthalen-1-yl]urea |
| 190 | 1-[5-tert-butyl-2-methoxy-pyridin-3-yl]-3-[4-(6-morpholin-4-yl- |
| | methyl6-pyridin-3-yl)naphthalen-1-yl]urea |
| 191 | 2,2,2-trifluoroethanesulfonic acid (5-tert-butyl-2-methoxy-3-{3-[4- |
| | (6-morpholin-4-ylmethyl-pyridin-3-yl)-naphthalen-1-yl]-ureido}- |
| | phenyl)-amide |
| 192 | N-(5-{4-[3-(5-tert-butyl-2-methyl-phenyl)-ureido]-naphthalen-1- |
| | yl}-pyrazin-2-yl)-methanesulfonamide |
| 193 | 1-[5-tert-butyl-2-methoxy-phenyl]-3-[4-(6-{[bis-(2-cyano-ethyl)- |
| | amino]-methyl}-pyridin-3-yl)naphthalen-1-yl]urea |
| 194 | 1-[5-tert-butyl-2-methoxy-phenyl]-3-{4-[6-(4-methyl-piperazin-1- |
| | ylmethyl)pyridin-3-yl]naphthalen-1-yl}urea |
| 195 | 1-[5-tert-butyl-2-methoxy-phenyl]-3-[4-(6-thiomorpholin-4-yl- |
| | methyl-pyridin-3-yl)naphthalen-1-yl]urea |
| 196 | 1-[5-tert-butyl-2-methoxy-phenyl]-3-{4-[6-(2,6-dimethyl-piperidin- |
| | 1-ylmethyl)-pyridin-3-yl]naphthalen-1-yl}urea |
| 197 | 1-[5-tert-butyl-2-methoxy-phenyl]-3-{4-[6-(1-oxo-tetrahydropyran- |
| | 4-ylamino)-pyridin-3-yl]naphthalen-1-yl}urea |
| 198 | 1-[5-tert-butyl-2-methoxy-phenyl]-3-{4-[6-(tetrahydropyran-4- |
| | ylamino)-pyridin-3-yl]naphthalen-1-yl}urea |
| 199 | 1-[5-tert-butyl-2-methoxy-phenyl]-3-[4-[6-{[(2-cyano-ethyl)- |
| | tetrahydro-furan-2-ylmethyl)-amino]-methyl}-pyridin-3- |
| | yl)naphthalen-1-yl}urea |
| 200 | 1-[5-tert-butyl-2-methoxy-phenyl]-3-{4-[6-(2-methoxymethyl- |
| | morpholin-4-ylmethyl)-pyridin-3-yl]naphthalen-1-yl}urea |
| 201 | 1-[5-tert-butyl-2-methoxy-phenyl]-3-{4-[6-(2-methyl-3-oxo- |
| | piperazin-1-ylmethyl)-pyridin-3-yl]naphthalen-1-yl}urea |
| 202 | 1-(5-{4-[3-(5-tert-butyl-2-methoxy-phenyl)-ureido]-naphthalen-1- |
| | yl}-pyridin-2-ylmethyl)-piperidine-3-carboxylic acid amide |
| 203 | 1-[5-tert-butyl-2-methoxy-phenyl]-3-{4-[6-(1-oxo-thiomorpholin-4- |
| | ylmethyl)-pyridin-3-yl]naphthalen-1-yl}urea |
| 204 | 1-(3,3-dimethyl-2-oxo-2,3-dihydro-1H-indol-5-yl)-3-[4-(6- |
| | morpholin-4-ylmethyl-pyridin-3-yl)-naphthalen-1-yl]urea |
| 205 | 1-[5-tert-butyl-2-methoxy-phenyl]-3-{4-[6-(3-oxo-piperazin-1- |
| | ylmethyl)-pyridin-3-yl]naphthalen-1-yl}urea |
| 206 | 1-[5-tert-butyl-2-methoxy-phenyl]-3-(4-{6-[(tetrahydro-furan-3- |
| | ylamino)methyl]-pyridin-3-yl}naphthalen-1-yl}urea |
| 207 | 1-[5-tert-butyl-2-methoxy-phenyl]-3-[4-(6-{[(2-cyano-ethyl)- |
| | pyridin-3-ylmethyl-amino]-methyl}-pyridin-3-yl)naphthalen-1- |
| | yl]urea |
| 208 | 1-[5-tert-butyl-2-methoxy-phenyl]-3-{4-[6-(2-oxa-5-aza- |
| | bicyclo[2.2.1]hept-5-ylmethyl)-pyridin-3-yl]naphthalen-1-yl}urea |
| 209 | 1-[5-tert-butyl-2-methoxy-phenyl]-3-{4-[6-(2,6-dimethyl- |
| | morpholin-4-ylmethyl)pyridin-3-yl]naphthalen-1-yl}urea |
| 210 | 1-[5-tert-butyl-2-methoxy-phenyl]-3-(4-{6-[4-(3-methoxy-phenyl)- |
| | 1-piperazin-1-ylmethyl]-pyridin-3-yl}naphthalen-1-yl)urea |
| 211 | 1-[5-tert-butyl-2-methoxy-phenyl]-3-{4-[6-(morpholine-4- |
| | carbonyl)-pyridin-3-yl]naphthalen-1-yl}urea |
| 212 | 1-[5-tert-butyl-2-methoxy-phenyl]-3-[4-(5-morpholin-4-ylmethyl- |
| | pyrazin-2-yl)-naphthalen-1-yl}urea |
| 213 | 1-[6-tert-butyl-3-oxo-3,4-dihydro-2H-benzo[1,4]oxazin-8-yl]-3-[4- |
| | (6-morpholin-4-ylmethyl-pyridin-3-yl) naphthalen-1-yl]urea |
| 214 | 1-[3-amino-5-tert-butyl-2-methoxy-phenyl]-3-[4-(6-morpholin-4-yl- |
| | methyl-pyridin-3-yl)naphthalen-1-yl]urea |
| 215 | N-(5-{4-[3-(5-tert-butyl-2-methoxy-phenyl)-ureido]-naphthalen-1- |
| | yl}pyridin-2-yl)-acetamide |
| 216 | N-(5-tert-butyl-2-methoxy-3-{3-[4-(6-morpholin-4-ylmethyl- |
| | pyridin-3-yl)-naphthalen-1-yl]-ureido}-phenyl)-N- |
| | methyl-acetamide |
| 217 | N-(5-tert-butyl-2-methoxy-3-{3-[4-(6-morpholin-4-ylmethyl- |
| | pyridin-3-yl)-naphthalen-1-yl]-ureido}-phenyl)-2,2,2- |
| | trifluoro-acetamide |
| 218 | 1-[5-tert-butyl-2-methoxy-phenyl]-3-{4-[6-(pyridin-3-yloxy)- |
| | pyridin-3-yl]naphthalen-1-yl}urea |
| 219 | [4-(6-morpholin-4-ylmethyl-pyridin-3-yl)-naphthalen-1-yl]- |
| | carbamic acid 3-tert-butyl-phenyl ester |
| 220 | N-(5-tert-butyl-2-methoxy-3-{3-[4-(6-morpholin-4-ylmethyl- |
| | pyridin-3-yl)-naphthalen-1-yl]-ureido}-phenyl)- |
| | methanesulfonamide |
| 221 | 1-[2-carbomethoxy-5-tert-butyl-3-thienyl]-3-[4-(2-morpholin-4-yl- |
| | ethoxy)naphthalen-1-yl]urea |
| 222 | 1-[2-methylcarbamoyl-5-tert-butyl-3-thienyl]-3-[4-(2-morpholin-4- |
| | yl-ethoxy)naphthalen-1-yl]urea |
| 223 | 1-[2-methylcarbamoyl-5-tert-butyl-3-thienyl]-3-[4-(2-(trans-2,6- |
| | dimethylmorpholin-4-yl)ethoxy)naphthalen-1-yl]urea |
| 224 | 1-[2-methylcarbamoyl-5-tert-butyl-3-thienyl]-3-[4-(2-(2- |
| | dimethylaminomethyldimethylmorpholin-4-yl)ethoxy)naphthalen- |
| | 1-yl]urea |
| 225 | 1-[2-carbomethoxy-5-tert-butyl-3-thienyl]-3-[4-(2-(1- |
| | oxothiomorpholin-4-yl)ethoxy)naphthalen-1-yl]urea |
| 226 | 1-[2-methylcarbamoyl-5-tert-butyl-3-thienyl]-3-[4-(2-(1- |
| | oxothiomorpholin-4-yl)ethoxy)naphthalen-1-yl]urea |
| 227 | 1-[3-carbomethoxy-5-tert-butyl-2-thienyl]-3-[4-(2-(1- |
| | oxothiomorpholin-4-yl)ethoxy)naphthalen-1-yl]urea |
| 228 | 1-[2-carbomethoxy-5-tert-butyl-3-thienyl]-3-[4-(2- |
| | (tetrahydropyran-4-yl)ethoxy)naphthalen-1-yl]urea |
| 229 | 1-[2-methylcarbamoyl-5-tert-butyl-3-thienyl]-3-[4-(2- |
| | (tetrahydropyran-4-yl)ethoxy)naphthalen-1-yl]urea |
| 230 | 1-[3-carbomethoxy-5-tert-butyl-2-thienyl]-3-[4-(2- |
| | (tetrahydropyran-4-yl)ethoxy)naphthalen-1-yl]urea |
| 231 | 1-[2-carbomethoxy-5-tert-butyl-3-thienyl]-3-[4-(2-(1-oxo- |
| | tetrahydrothiophen-3-yl)ethoxy)naphthalen-1-yl]urea |
| 232 | 1-[2-methylcarbamoyl-5-tert-butyl-3-thienyl]-3-[4-(2-(1-oxo- |
| | tetrahydrothiophen-3-yl)ethoxy)naphthalen-1-yl]urea |
| 233 | 1-[2-carbomethoxy-5-tert-butyl-3-thienyl]-3-[4-(pyridin-4-yl- |
| | methoxy)naphthalen-1-yl]urea |
| 234 | 1-[2-methylcarbamoyl-5-tert-butyl-3-thienyl]-3-[4-(pyridin-4- |
| | yl)methoxy)naphthalen-1-yl]urea |
| 235 | 1-[2-carbomethoxy-5-tert-butyl-3-thienyl]-3-[4-(2-imidazol-1-yl- |
| | ethoxy)naphthalen-1-yl]urea |
| 236 | 1-[2-carbomethoxy-5-tert-butyl-3-thienyl]-3-[4-(2-(3,4- |
| | dimethoxyphenyl)-ethoxy)naphthalen-1-yl]urea |
| 237 | 1-[2-methylcarbamoyl-5-tert-butyl-3-thienyl]-3-[4-(2-(3,4- |
| | dimethoxyphenyl)ethoxy)naphthalen-1-yl]urea |
| 238 | 1-[2-carbomethoxy-5-tert-butyl-3-pyrrolyl]-3-[4-(2-morpholin-4-yl- |
| | ethoxy)naphthalen-1-yl]urea |
| 239 | 1-[2-methylcarbamoyl-5-tert-butyl-3-pyrrolyl]-3-[4- |
| | (2-morpholin-4-yl-ethoxy)naphthalen-1-yl]urea |
| 240 | 1-[1-methyl-2-carbomethoxy-5-tert-butyl-3-pyrrolyl]-3-[4-(2- |
| | morpholin-4-yl-ethoxy)naphthalen-1-yl]urea |
| 241 | 1-[2-carbomethoxy-5-tert-butyl-3-pyrrolyl]-3-[4-(2-(1- |
| | oxothiomorpholin-4-yl)ethoxy)naphthalen-1-yl]urea |
| 242 | 1-[2-methylcarbamoyl-5-tert-butyl-3-pyrrolyl]-3-[4-(2-(1- |
| | oxothiomorpholin-4-yl)ethoxy)naphthalen-1-yl]urea |
| 243 | 1-[1-methyl-2-carbomethoxy-5-tert-butyl-3-pyrrolyl]-3-[4-(2-(1- |
| | oxothiomorpholin-4-yl)ethoxy)naphthalen-1-yl]urea |
| 244 | 1-[2-carbomethoxy-5-tert-butyl-3-pyrrolyl]-3-[4-(2- |
| | (tetrahydropyran-4-yl)ethoxy)naphthalen-1-yl]urea |
| 245 | 1-[2-methylcarbamoyl-5-tert-butyl-3-pyrrolyl]-3-[4-(2- |
| | (tetrahydropyran-4-yl)ethoxy)naphthalen-1-yl]urea |
| 246 | 1-[2-carbomethoxy-5-tert-butyl-3-pyrrolyl]-3-[4-(2-(1-oxo- |
| | tetrahydrothiophen-3-yl)ethoxy)naphthalen-1-yl]urea |
| 247 | 1-[2-methylcarbamoyl-5-tert-butyl-3-pyrrolyl]-3-[4-(2-(1-oxo |
| | tetrahydrothiophen-3-yl)ethoxy)naphthalen-1-yl]urea |
| 248 | 1-[1-methyl-2-carbomethoxy-5-tert-butyl-3-pyrrolyl]-3-[4-(2-(1- |
| | tetrahydrothiaphen-3-yl)ethoxy)naphthalen-1-yl]urea |
| 249 | 1-[2-carbomethoxy-5-tert-butyl-3-pyrrolyl]-3-[4-(pyridin-4-yl- |
| | methoxy)naphthalen-1-yl]urea |
| 250 | 1-[2-methylcarbamoyl-5-tert-butyl-3-pyrrolyl]-3-[4-(pyridin-4-yl- |
| | methoxy)naphthalen-1-yl]urea |
| 251 | 1-[2-carbomethoxy-5-tert-butyl-3-pyrrolyl]-3-[4-(pyridin-4-yl- |
| | ethoxy)naphthalen-1-yl]urea |
| 252 | 1-[2-carbomethoxy-5-tert-butyl-3-pyrrolyl]-3-[4-(2-imidazol-1-yl- |
| | ethoxy)naphthalen-1-yl]urea |
| 253 | 1-[2-carbomethoxy-5-tert-butyl-3-pyrrolyl]-3-[4-(2-(3,4- |
| | dimethoxyphenyl)ethoxy)naphthalen-1-yl]urea |
| 254 | 1-[2-methylcarbamoyl-5-tert-butyl-3-pyrrolyl]-3-[4-(2-(3,4- |
| | dimethoxyphenyl)ethoxy)naphthalen-1-yl]urea |
| 255 | 1-(5-tert-butyl-2-methoxyphenyl)-3-[4-(2-methoxy-pyridin-4-yl- |
| | oxy)-naphthalen-1-yl]urea |
| 256 | 1-(5-tert-butyl-2-methoxyphenyl)-3-[4-(2-methyl-pyridin-4-yl-oxy)- |
| | naphthalen-1-yl]urea |
| 257 | 1-(5-tert-butyl-2,3-dimethoxyphenyl)-3-[4-(2-methoxy-pyridin-4- |
| | yl-oxy)-naphthalen-1-yl]urea |
| 258 | 5-tert-butyl-2-methoxy-3-{3-(pyridin-4-yl-oxy)naphthalen-1- |
| | yl]ureido}benzamide |
| 259 | Morpholine carboxylic acid (5-tert-butyl-2-methoxy-3-{3-[- |
| | (pyridin-4-yloxy)naphthalen-1-yl}ureido}phenyl)amide |
| 260 | N-(5-tert-butyl-2-methoxy-3-{3-[-(pyridin4-yl-oxy)naphthalen-1- |
| | yl]ureido}phenyl)acetamide |
| 261 | 3-(5-tert-butyl-2-methoxy-3-{3-[-(pyridin-4-yl-oxy)naphthalen-1- |
| | yl]ureido}phenyl)-1,1-dimethylurea |
| 262 | 1-(5-tert-butyl-2,3-dimethoxy-phenyl)-3-[4-(2-amino-pyridin-4- |
| | yloxy)-naphthalen-1-yl]urea |
| 263 | 1-(5-tert-butyl-2,3-dimethoxyphenyl)-3-[4-(2-methylamino-pyridin- |
| | 4-yloxy]-naphthalen-1-yl}urea |
| 264 | 1-(5-tert-butyl-2,3-dimethoxyphenyl)-3-{4-[2-(1-phenyl- |
| | ethylamino)-pyridin-4-yloxy]-naphthalen-1-yl}urea |
| 265 | 1-(5-tert-butyl-2-methoxy-pyridin-3-yl)-3-[4-(2-amino-pyridin-4- |
| | yloxy)-naphthalen-1-yl]urea |
| 266 | 1-(5-tert-butyl-2-methoxy-pyridin-3-yl)-3-[4-(2-methylamino- |
| | pyridin-4-yloxy]-naphthalen-1-yl}urea |
| 267 | 1-(5-tert-butyl-2-methoxy-pyridin-3-yl)3-{4-[2-(1-phenyl- |
| | ethylamino)-pyridin-4-yloxy]-naphthalen-1-yl}urea |
| 268 | 1-(5-tert-butyl-2-methyl-phenyl)-3-[4-(2-aminopyridin-4-yl-oxy)- |
| | naphthalen-1-yl]urea |
| 269 | 1-(5-tert-butyl-2-morpholin-4-yl-phenyl)-3-[4-(2-morpholin-4-yl- |
| | ethoxy)-naphthalen-1-yl]urea |
| 270 | 1-(5-tert-butyl-2-methoxyphenyl)-3-[4-(2-methylpyridin-4-yl-oxy)- |
| | naphthalen-1-yl]urea |
| 271 | 1-(5-tert-butyl-2,3-dimethoxyphenyl)-3-[4-(2-methoxypyridin-4-yl- |
| | oxy)-naphthalen-1-yl]urea |
| 272 | 5-tert-butyl-2-methoxy-3-{3-[-(pyridin-4-yl-oxy)naphthalen-1- |
| | yl]ureido}benzamide |
| 273 | Morpholine-4-carboxylic acid (5-tert-butyl-2-methoxy-3-{3-[- |
| | (pyridin-4-yl-oxy)naphthalen-1-yl]ureido}phenyl)amide |
| 274 | N-(5-tert-butyl-2-methoxy-3-{3-[-(pyridin-4-yl-oxy)naphthalen-1- |
| | yl]}ureido}phenyl)acetamide |
| 275 | 3-(5-tert-butyl-2-methoxy-3-{3-[-(pyridin-4-yl-oxy)naphthalen-1- |
| | yl]ureido}phenyl)-1,1-dimethylurea |
| 276 | 1-(5-tert-butyl-2,3-dimethoxy-phenyl)-3-[4-(2-amino-pyridin-4- |
| | yloxy)-naphyhalen-1-yl]urea |
| 277 | 1-(5-tert-butyl-2,3-dimethoxy-phenyl)-3-[4-(2-methylamino- |
| | pyridin-4-yloxy)-naphthalen-1-yl]urea |
| 278 | 1-(5-tert-butyl-2,3-dimethoxy-phenyl)-3-[4-(2-phenyl-ethylamino- |
| | pyridin-4-yloxy)-naphthalen-1-yl]urea |
| 279 | 1-(5-tert-butyl-2-methoxy-pyridin-3-yl)-3-[4-(2-amino-pyridin-4- |
| | yloxy)-naphthalen-1-yl]urea |
| 280 | 1-(5-tert-butyl-2-methoxy-pyridin-3-yl)-3-[4-(2-methylamino- |
| | pyridin-4-yloxy)-naphthalen-1-yl]urea |
| 281 | 1-(5-tert-butyl-2-methoxy-pyridin-3-yl)-3-{4-[2-(1-phenyl- |
| | ethylamino)-pyridin-4-yloxy)-naphthalen-1-yl]urea |
| 282 | 1-(5-tert-butyl-2-methyl-phenyl)-3-[4-(2-aminopyridin-4-yloxy)- |
| | naphthalen-1-yl]urea |
| 283 | 1-(5-tert-butyl-2-morpholin-4-yl-phenyl)-3-[4-(2-morpholin-4-yl- |
| | ethoxy)-naphthalen-1-yl]urea |

b) salts of the ureas within the above group,
c) prodrugs of the ureas within the above group, and
d) isolated stereoisomers within the above group.

28. A method as in claim 27 wherein the infectious diseases are selected from the group consisting of tuberculosis, Helicobacter pylori infection during peptic ulcer disease, Chaga's disease resulting from Trypanosoma cruzi infection, effects of Shiga-like toxin resulting from E. coli infection, effects of enterotoxin A resulting from Staphylococcus infection, meningococcal infection and infections from Borrelia burgdorferi, Treponema pallidum, cytomegalovirus, influenza virus, Theiler's encephalomyelitis virus, and the human immunodeficiency virus (HIV).

29. A method as in claim 27 wherein the compound administered is within a pharmaceutical composition which additionally comprises a physiologically acceptable carrier.

30. A method for treating or preventing a disease in a human or other mammal regulated by tyrosine kinase,(associated with an aberration in the tyrosine, kinase signal transduction pathway) comprising administering to a human or other mammal in need thereof 1-[5-tert-butyl-2-p-tolyl-2H-pyrazol-3-yl]-3-[4-(2-morpholin-4-ylethoxy)naphthalen-1-yl]-urea, a salt thereof, a prodrug thereof or an isolated stereoisomer thereof.

31. A method as in claim 1 wherein the disease that is treated or prevented is a KDR-mediated disorder.

32. A method as in claim 1 wherein the disease that is treated or prevented is a Flk-1 mediated disorder.

33. A method of regulating tyrosine kinase signal transduction comprising administering to a human or other mammal 1-[5-tert-butyl-2-p-tolyl-2H-pyrazol-3-yl]-3-[4-(2-morpholin-4-ylethoxy)naphthalen-1-yl]-urea, a salt thereof, a prodrug thereof or an isolated stereoisomer thereof.

34. The method of claim 30, wherein said disease is cancer.

35. A method for treating or preventing a raf mediated disease in a human or other mammal comprising administering to a human or other mammal in need thereof 1-[5-tert-butyl-2-p-tolyl-2H-pyrazol3-yl- ]-3-[4-(2-morpholin-4-ylethoxy)naphthalen-1-yl]-urea, a salt thereof, a prodrug thereof or an isolated stereoisomer thereof.

36. A method as in claim 35 wherein the raf mediated disease is cancer.

37. A method for treating and preventing hyper-proliferative disorders comprising administering to a human or other mammal 1-[5-tert-butyl-2-p-tolyl-2H-pyrazol-3-yl]-3-[4-(2-morpholin-4-ylethoxy)naphthalen-1-yl]-urea, a salt thereof, a prodrug thereof or an isolated stereoisomer thereof.

38. A method as in claim 37 wherein the hyper-proliferative disorder that is treated is a solid cancer, carcinoma of the lungs, carcinoma of the pancreas, carcinoma of the thyroid, carcinoma of the bladder, carcinoma of the colon, a myeloid disorder, or an adenoma.

39. A method as in claim 37 wherein the hyper-proliferative disorder that is treated is a raf kinase-mediated cancer.

40. A method as in claim 37 wherein the hyper-proliferative disorder that is treated is human colon, gastric, lung, pancreatic, ovarian, prostate, leukemia, melanoma, hepatocellular, renal, head and neck, glioma, or mammary cancer.

41. A method as in claim 37 wherein at least one additional anti-proliferative agent is administered with the 1-[5-tert-butyl-2-p-tolyl-2H-pyrazol-3-yl]-3-[4-(2-morpholin-4-ylethoxy)naphthalen-1-yl]-urea, a salt thereof, a prodrug thereof or an isolated stereoisomer thereof either simultaneously in the same pharmaceutical composition or separately, in separate pharmaceutical compositions.

42 A method as in claim 41 wherein the additional anti-proliferative agent is selected from the group consisting of asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, vindesine,
aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, oxaliplatin, pentostatin, N-phosphonoacetyl-L-aspartate (PALA), plicamycin, semustine, teniposide, testosterone propionate, thiotepa, trimethylmelamine, uridine, vinorelbine, epothilone, gemcitabine, taxotere, BCNU, CCNU, DTIC, 5-fluorouarcil, herceptin and actinomycin D.

43. A method as in claim 41 wherein the one or more administered compounds and the additional anti-proliferative agent are administered separately in separate formulations.

42. A method as in claim 37 wherein at least one cytotoxic agent or cytostatic chemotherapeutic agent is administered with the 1-[5-tert-butyl-2-p-tolyl-2H-pyrazol -3-yl]-3-[4-(2morpholin-4-ylethoxy)naphthalen-1-yl]-urea, a salt thereof, a prodrug thereof or an isolated stereoisomer thereof, either simultaneously in the same pharmaceutical composition or separately, in separate pharmaceutical compositions,
wherein the cytotoxic agent or cytostatic chemotherapeutic agent is selected from the group consisting of DNA topoisomerase I and II inhibitors, DNA intercalators, alkylating agents, microtubule disruptors, hormone and growth factor receptor agonists or antagonists, other kinase inhibitors and antimetabolites.

43. A method as in claim 37 wherein the hyper-proliferative disorder that is treated is human pancreatic, lung, colon, ovarian, prostate, leukemia, melanoma, hepatocellular, renal, head and neck, glioma, or mammary carcinoma.

44. A kit comprising a separate dose of 1-[5-tert-butyl-2-p-tolyl-2H-pyrazol-3-yl]-3-[4-(2-morpholin-4-ylethoxy)naphthalen-1-yl]-urea, a salt thereof, a prodrug thereof or an isolated stereoisomer thereof and a separate dose of an additional anti-proliferative agent.
